SEQUENCE LISTING (2 sequences)

SEQ ID NO: 1 - DNA, 18 bp, Artificial sequence
  Synthetic oligonucleotide
ggtgccgccagctgggag

SEQ ID NO: 2 - DNA, 18 bp, Artificial sequence
  Synthetic oligonucleotide
gcgtgctgaccacacgct

(19) United States
(12) Patent Application Publication
(10) Pub. No.: US 2022/0251516 A1
(43) Pub. Date: Aug. 11, 2022

(54) METHODS FOR THE PRODUCTION OF MULTIPLE LINEAGES FROM INDUCED PLURIPOTENT STEM CELLS USING CHARGED SURFACES

(71) Applicants: FUJIFILM Cellular Dynamics, Inc., Madison, WI (US); FUJIFILM Holdings America Corporation, Valhalla, NY (US)

(72) Inventors: Deepika RAJESH, Madison, WI (US); Christie MUNN, Madison, WI (US); Sarah BURTON, Madison, WI (US); Madelyn GOEDLAND, Madison, WI (US); Michael McLACHLAN, Madison, WI (US); Abbey MUSINSKY, Madison, WI (US); Kwi Hye KIM, Madison, WI (US); Michael HANCOCK, Madison, WI (US); Makiko OHSHIMA, Madison, WI (US); Anne STROUSE, Madison, WI (US); Sarah DICKERSON, Madison, WI (US)

(73) Assignees: FUJIFILM Cellular Dynamics, Inc., Madison, WI (US); FUJIFILM Holdings America Corporation, Valhalla, NY (US)

(21) Appl. No.: 17/619,096

(22) PCT Filed: Jun. 15, 2020
(86) PCT No.: PCT/US2020/037790

§ 371 Date: Dec. 14, 2021

Related U.S. Application Data
(60) Provisional application No. 63/038,564, filed on Jun. 12, 2020.
(60) Provisional application No. 62/865,806, filed on Jun. 24, 2019.
(60) Provisional application No. 62/861,640, filed on Jun. 14, 2019.

(51) Int. Cl. C12N 5/074; C12N 5/00

(52) U.S. Cl. CPC C12N 5/0696; C12N 2533/32; C12N 2506/45; C12N 5/0075

(57) ABSTRACT

The present disclosure provides methods of producing progenitor cells from induced pluripotent stem cells, wherein the progenitor cells comprise endothelial cells, pericytes, brain microvascular endothelial cells (BMECs), mesenchymal stem cells (MSCs), hematopoietic precursor cells (HPCs), microglia or neural precursor cells (NPCs).

This application claims the benefit of U.S. Provisional Patent Application Nos. 62/861,640, filed Jun. 14, 2019, 62/865,806, filed Jun. 24, 2019, and 63/038,564, filed Jun. 12, 2020, the entirety of which are incorporated herein by reference.

### BACKGROUND

### 1. Field

The present invention relates generally to the field of molecular biology and medicine. More particularly, it concerns methods of differentiating induced pluripotent stem cells.

### 2. Description of Related Art

Human pluripotent stem cells (hPSCs) provide powerful resources for application in regenerative medicine and pharmaceutical development. In the past decade, various methods have been developed for generating multiple lineages from iPSCs using multiple growth components, extracellular matrices, and/or feeder layers to offer a potentially powerful tool for improving in vitro models and investigating the early stages of development of different lineages.

Efficient in vitro differentiation into specific cell types is key for its potential use for disease modeling and drug screening. Key success factors for lineage specific differentiation include the use of defined conditions, a good phenotypic and functional characterization of end stage cells, reproducibility of the process length of differentiation, and cost. Accordingly, there remains a need in the art for efficient methods for lineage specific differentiation of human pluripotent stem cells.

### SUMMARY

Certain embodiments of the present disclosure provides methods and compositions for an in vitro method for differentiating induced pluripotent stem cells (iPSCs) comprising: (a) culturing the iPSCs on a charged surface in the absence of extracellular matrix proteins; and (b) differentiating the iPSCs to endothelial cells, mesenchymal stem cells (MSCs), or hematopoietic precursor cells (HPCs).

In some aspects, the charged surface is positively charged. In certain aspects, the positively charged surface is an amine surface or Poly L Lysine surface. In specific aspects, the positively charged surface comprise nitrogen-containing functional groups. In other aspects, the charged surface is negatively charged. In particular aspects, the negatively charged surface is a carboxyl surface. In certain aspects, the negatively charged surface comprise oxygen-containing functional groups. In some aspects, the charged surface is a polymeric surface. For example, the polymeric surface is a polystyrene surface. In certain aspects, the charged surface comprises positively charged groups and negatively charged groups. In some aspects, the positively charged groups are nitrogen-containing groups and the negatively charged groups are oxygen-containing groups.

In certain aspects, the iPSCs are cultured in serum free defined media. In some aspects, differentiating comprises culturing in the presence of blebbistatin or a ROCK inhibitor, such as H1152. In particular aspects, the method is free of or essentially free of extracellular matrix proteins, such as laminin, fibronectin, vitronectin, MATRIGEL™, tenascin, entactin, thrombospondin, elastin, gelatin, or collagen.

In additional aspects, the method further comprises engineering the iPSCs to have disrupted expression of TREM2, MeCP2, and/or SCNA prior to step (a). In particular aspects, engineering comprises using a TAL nuclease to introduce indels in exon 2 of TREM2.

In some aspects, the disrupted expression of MeCP2 is further defined as a truncated mutant of MeCP2 protein. In certain aspects, the disrupted expression is due to a missense point mutation, such as A53T.

In some aspects, the method comprises differentiating the progenitor cells to endothelial cells. In certain aspects, step (a) comprises culturing on an amine surface to generate progenitor cells and step (b) comprises culturing on a carboxyl surface in the presence of endothelial differentiation media to produce endothelial cells. In particular aspects, the endothelial cells are positive for CD31.

In further aspects, the method further comprises differentiating the endothelial cells to brain microvascular endothelial cells (BMECs).

In some aspects, the method further comprises differentiating the endothelial cells to lymphatic endothelial cells.

In certain aspects, the method comprises differentiating the progenitor cells to MSCs. In specific aspects, differentiating comprises culturing the progenitor cells on an amine surface in the presence of MSC media. In some aspects, the MSCs are positive for CD73, CD44, and CD105. In certain aspect, at least 90% of the differentiated cells are positive for CD73.

In additional aspects, the method further comprises differentiating the MSCs to pericytes. In certain aspects, the MSCs are cultured in the presence of pericyte medium in the absence of extracellular proteins. In some aspects, the pericytes are positive for NG2, PDGFRβ, and CD146.

In some aspects, the method comprises differentiating the progenitor cells to HPCs. In certain aspects, the method further comprises differentiating the HPCs to microglia. In specific aspects, differentiating comprises culturing the HPCs on a neutrally charged surface or ultralow attachment surface in the presence of microglia differentiation media. In certain aspects, the microglia differentiation media comprises IL34, TGF, and MCSF. In some aspects, differentiating comprises culture at normoxia. In some aspects, differentiating is for 20-25 days. In particular aspects, the microglia are positive for CD45, CD11b, and CD33. In certain aspects, at least 50% (e.g., 51%, 52%, 53%, 54%, 55%, 56%, 57%, 58%, 59%, 60%, 65%, 70%, 75%, 80%, 85%, 90%, 50-60%, 60-70%, or 80-90%) of the differentiated cells are positive for CD11b. In some aspects, at least 90% (e.g., 91%, 92%, 93%, 94%, 95%, 96%, 97%, 98%, 99%, or 100%) of the differentiated cells are positive for CD33.

In certain aspects, the method does not comprise purification of the cells. In some aspects, purification is further defined as performing MACS.

In particular aspects, the method is good-manufacturing practice (GMP) compliant. In some aspects, the method is performed under hypoxic conditions. In specific aspects, the iPSCs are human

In another embodiment, there is provided a composition comprising a microglia cell population at least 90% (e.g., 91%, 92%, 93%, 94%, 95%, 96%, 97%, 98%, 99%, 100%, 90-93%, 93-96%, or 96-100%) positive for P2RY12, CX3CR1, TMEM119, IBA-1 and TREM2. In some aspects, the microglia cell population is produced by the method of the embodiments. In some aspects, the microglia cell population is generated from disease associated iPSC donors with TREM2, APOE, CD33, BIN, ABCA7, SNPS or genotypes associated with neurodegeneration. In certain aspects, the microglia cell population has disrupted expression of TREM2, MeCP2, and/or SCNA. In some aspects, the disrupted expression of TREM2 is further defined as a homozygous knockout of TREM2 expression. In certain aspects, the disrupted expression of MeCP2 is further defined as a truncated mutant of MeCP2 protein. In some aspects, the disrupted expression of SCNA is due to a missense point mutation, such as A53T.

A further embodiment provides a method for screening a test compound comprising introducing the test compound to a microglia cell population of the present embodiments. In some aspects, the microglia cell population is further introduced to amyloid-beta. In certain aspects, the microglia cell population is further introduced to LPS.

Another embodiment provides a composition comprising a pericyte cell population produced by the method of the present embodiments.

In yet another embodiment, there is provided herein a blood-brain-barrier model comprising microglia, pericytes, and BMECs produced by the present embodiments.

A further embodiment provides a method for generating microglia comprising: (a) differentiating iPSCs to HPCs; and (b) sorting the HPCs for CD34-positive cells; and (c) culturing the HPCs in Microglia Differentiation Medium, thereby generating a population of microglia. In some aspects, the HPCs are differentiated according to the present embodiments. In certain aspects, sorting comprises using CD34 magnetic beads. In particular aspects, the method does not comprise sorting HPCs for CD43-positive cells. In particular aspects, the method does not comprise ECM proteins.

In some aspects, the Microglia Differentiation Medium comprises IL-34, TGFβ1, or M-CSF. In certain aspects, the Microglia Differentiation Medium comprises 200 ng/mL IL-34, 100 ng/mL TGFβ1, and 50 ng/mL M-CSF. In some aspects, the cells are fed with Microglia Differentiation Medium every 48 hours.

In particular aspects, at least 90% (e.g., 91%, 92%, 93%, 94%, 95%, 96%, 97%, 98%, 99%, 100%, 90-93%, 93-96%, or 96-100%) of the cells in the population of microglia are TREM-positive. In some aspects, at least 10% (e.g., 15%, 20%, 25%, 30%, 10-15%, 15-20%, or 20-30%) of the HPCs are differentiated to microglia.

In certain aspects, the culturing of step (b) is performed in a 96 well format. In certain aspects, the culturing of step (b) is performed on a charged surface. In some aspects, the charged surface is positively charged. For example, the positively charged surface is an amine surface. In other aspects, the charged surface is negatively charged. For example, the negatively charged surface is a carboxyl surface.

In additional aspects, the method further comprises maturing the population of microglia in media comprising CD200 and/or fractalkine. In some aspects, the method further comprises cryopreserving the microglia.

In some aspects, the HPCs are differentiated from iPSCs engineered to have disrupted expression of TREM2. In certain aspects, engineering comprises using a TAL nuclease.

In certain aspects, the cryopreserved microglia retain phagocytic function towards pHrodo bacterial particles. In particular aspects, the cryopreserved microglia can mature post thaw and respond to stimulants and secrete interleukins, chemokines and immune modulating ligands in the supernatant media.

A further embodiment provides an in vitro method for producing neural progenitor cells (NPCs) from iPSCs comprising (a) pre-conditioning iPSCs in media comprising a glycogen synthase kinase 3 (GSK3) inhibitor; (b) differentiating the iPSCs to

NPCs, wherein the method does not comprise inhibition of SMAD signaling.

In some aspects, the iPSCs are maintained under hypoxic conditions prior to step (a). In certain aspects, the iPSCs are seeded in the presence of a ROCK inhibitor and then cultured in the absence of a ROCK inhibitor prior to step (a).

In certain aspects, the GSK3 inhibitor is CH1R99021, BIO, or SB-216763. In particular aspects, the GSK3 inhibitor is CH1R99021, such as at a concentration of 1 μM, 2 μM, 3 μM, 4 μM, or 5 μM, particularly 3 μM. In some aspects, the pre-conditioning is for 2-4 days, such as 1, 2, or 3 days.

In some aspects, the iPSCs of step (a) and/or step (b) are cultured on an extracellular matrix (ECM) protein-coated surface. In certain aspects, the ECM protein is MATRIGEL™, laminin, or vitronectin. In specific aspects, the ECM protein is laminin or vitronectin.

In certain aspects, steps (a) and (b) are performed under normoxic conditions. In some aspects, differentiating comprises culturing the iPSCs on an ECM protein-coated surface. In certain aspects, the ECM protein is laminin or vitronectin. In some aspects, differentiating comprises culturing the iPSCs on an ultralow attachment plate or spinner flask in the presence of a ROCK inhibitor. In particular aspects, step (b) is performed for 5 to 10 days, such as 6 days, 7 days, 8 days, 9 days, or 10 days.

In additional aspects, the method further comprises detecting expression of Tra-162, CD56, CD15, Sox1, Nestin, β3 Microglobulin, and/or Pax-6 in the NPCs. In some aspects, at least 70% (e.g., 80%, 85%, 90%, 95%, 70-80%, 80-90%, or 90-100%) of the NPCs are positive for CD56.

In further aspects, the method comprises further differentiating the NPCs to astrocytes or neurons.

Another embodiment provides a method of screening for a neurodegenerative disease comprising detecting a level of soluble TREM2 in microglia conditioned media. In some aspects, detecting comprises performing an ELISA. In certain aspects, the microglia are derived from isogenically engineered iPSCs or a donor expressing disease-associated SNPs or mutations. In some aspects, the microglia are produced by the methods of the present embodiments or aspects thereof. In certain aspects, an increased level of soluble TEM as compared to a control detects a neurogenerative disease, such as Alzheimer's disease or multiple sclerosis.

A further embodiment provides a method for performing high-throughput screening to identify a therapeutic agent comprising contacting microglia produced by the method of the present embodiments or aspects thereof with a plurality of candidate agents and measuring cytokine and/or chemokine levels and/or amyloid beta phagocytic function.

In some aspects, the microglia are cryopreserved microglia derived from isogenically engineered iPSC lines, microglia derived from a donor expressing disease associated SNPs, or microglia derived from a donors expressing mutation associated with neurodegeneration. In some aspects, the cytokines and/or chemokines are selected from the group consisting of IL6, IL10, IL3, TNFα, IL13, CCL2/MCP-1, CCL20/MIP-3 α, CCL4/MIP-1βCCL5/RANTES, CX3CL1/Fractalkine, CXCL1/GROα, CXCL10/IP-10, CXCL2/GROβ, and IL-8/CXCL8.

Also provided herein is a co-culture comprising microglia of the present embodiments and aspects thereof and endothelial cells, pericytes, astrocytes, and/or neural precursor cells. Another embodiment provides the use of the co-culture to mimic human brain development.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic representation of the 2D and 3D HPC differentiation process.

FIG. 2: Schematic representation of the derivation of endothelial cells from day 6 HPCs derived from 2D or 3D HPC differentiation process.

FIG. 3: Characterization of endothelial cells generated using successive passage purification without the use of CD31⁺ MACS.

FIG. 4A-B: (FIG. 4A) Morphology of cells at the end of replate 3. The endothelial cells can be cryopreserved at the end of replate passage 2 or 3. (FIG. 4B) Media formulations used for derivation of endothelial cells.

FIGS. 5A-5D: (FIG. 5A) Overview of MSC differentiation process. (FIG. 5B) Media formulations for generating MSCs. (FIG. 5C) Schematic depicting tri lineage differentiation of MSCs to Adipocytes, Osteocytes and Cartilage. (FIG. 5D) Phenotypic characterization of day 6 MSC progenitor.

FIG. 6: Emergence of a pure population of MSCs on Amine surface. Cryopreserved day 6 HPCs or live cultures at the end of day 6 differentiation are placed in the presence of MSC media in the presence of 1 uM H1152 (or blebbistatin) on an Amine charged surface plates. The cultures were transitioned to normoxia and normal tissue culture plates at P4. The purity spec for MSCs was reached at P5.

FIG. 7: Cells stained for surface MSC markers CD73, CD44, CD105, CD49d, and absence of Endothelial markers CD31 and CD144. Cryopreserved day 6 HPCs or live cultures at the end of day 6 differentiation are placed in the presence of MSC media in the presence of 1 uM H1152 (or blebbistatin) on an Amine charged surface plates. The cultures were transitioned to normoxia and normal tissue culture plates at P5. The purity spec for MSCs was reached at P6.

FIGS. 8A-8C: (FIG. 8A) Tri-lineage potential. Various steps for generating adipocytes, osteocytes, and chondrocytes from MSCs. (FIG. 8B) Pictures demonstrating tri-lineage potential of MSCs, alizarin red staining for osteocytes, alcian blue for chondrocytes, and oil red O for adipocytes. (FIG. 8C) MSC plated at 1,000 cells/cm² in MSC Differentiation Medium and feed every other day for 10-14 days. Plates stained using crystal violet and total number of colonies counted.

FIGS. 9A-9H: Conversion of MSCs to pericytes. (FIG. 9A) Schematic overview of the process of converting iCell MSCs to iPSC-derived pericytes. (FIG. 9B) Media formulations for generating iPSC-derived pericytes. (FIG. 9C) Comparative flow cytometry for known pericyte markers PDGFRβ, NG2, and CD146 in iCell MSCs, iPSC-derived pericytes, and ScienCell primary human brain vascular pericytes (HBVPs). There was an absence of pericyte markers for iCell MSCs at thaw and gain of pericyte markers by the end of P1 in pericyte medium. iPSC-derived pericytes show greater purity of pericyte specific markers than primary HBVPs (FIG. 9D) Morphology of iPSC-derived MSC (P2), iPSC-derived pericyte (P1), and ScienCell primary HBVP morphology via bright field microscopy. (FIG. 9E) Table describing differences between PC1 and PC2 pericyte subtypes based on phenotype and marker expression. (FIG. 9F) iPSC-derived pericytes were stained via flow cytometry immediately post-thaw and five days post thaw for pericyte subtype specific markers CD274, VCAM1, Desmin, DLK1, and aSMA, as well as generic pericyte markers PDGFRβ, NG2, CD13, and CD146. iPSC-derived pericytes reveal the signature of contractile pericytes, subtype PC2. (FIG. 9G) IncuCyte live imaging system images of iPSC-derived pericytes in phagocytosis assay. iPSC-derived pericytes show observable phagocytic activity of S. aureus bioparticles above control levels (A) iPSC-derived pericyte alone control. (B) iPSC-derived pericyte +NucGreen Dead 488 (NG) reagent control. (C) iPSC-derived pericyte+S. aureus pHrodo Red BioParticles (BP). (D) iPSC-derived pericyte +NucGreen Dead 488 reagent (NG)+S. aureus pHrodo Red BioParticles (BP). All images taken from time point 36 days 16 hours post cell seeding. (FIG. 9H) Quantification of phagocytic activity via total red object integrated intensity analyzed by IncuCyte software.

FIG. 10A-10G: Generation of brain microvascular endothelial cells (BMECs). (FIG. 10A) Schematic overview of generating brain microvascular endothelial cells. (FIG. 10B) Composition of ECRA medium. (FIG. 10C) Flow cytometric analysis of BMECs by co expression of Glut1/CD31. (FIG. 10D) Immunohistochemical Staining of BMECs with P Glycoprotein (Green) expression on day 13. Nuclei was stained with Hoechst3342 and the images was captured at 200× magnification by ImageXpress (Molecular Devices, LLC). (FIG. 10E) Functional characterization of BMECs by measuring TEER values across days post plating. (FIG. 10F) Schematic overview of generating brain microvascular endothelial cells using an alternate method including a preconditioning step and plating on charged surfaces without using ECM. Description of the modified media to induce the generation of BMECs on charged surfaces. (FIG. 10G) Day 7 differentiating brain microvascular endothelial cells on different charged surfaces were harvested and the purity was quantified by staining for the presence of CD31, p glycoprotein/ and Glut-1 expression and the absence of pluripotency marker (TRA-181) expression.

FIG. 11: Scale up of HPCs using the 3D differentiation process and subsequent purification using CD34⁺Magnetic beads. A schematic description of scale up and sorting of HPCs using CD34 magnetic beads. The efficiency of the sorting process via manual and CliniMACs mediated separation is illustrated. The actual purity of the HPCs (measured by the percentage of CD34 positive cells in the sorted fraction) per run and the efficiency of the process is outlined.

FIG. 12: Media formulations for microglia differentiations.

FIGS. 13A-13B: (FG. 13A) A schematic description of derivation of microglia from CD34+ sorted HPC. HPCs were placed in microglia differentiation media MDM. The cultures were fed every 48 hours with MDM or 2× MDM. The cultures were split on day 12 of differentiation and the 2D differentiation was continued until day 23. The cells were harvested on day 23 and stained for the presence of purity markers for microglial cultures. The rest of the culture was cryopreserved. The purity of the microglia cultures was quantified before and after cryopreservation. (FIG. 13B) Microglia differentiation medium and microglia differentiation medium are depicted.

FIG. 14: Microglia purity assessment in the presence of MDM media before and after cryopreservation. Microglia cultures on day 23 differentiation were harvested and stained for the presence of microglia specific markers. The remaining cells were cryopreserved using a control rate freezer. The cryopreserved cells were thawed and stained for the presence of microglia specific markers. For both sets cell surface expression of CD45, CD33, TREM2, and CD11b (FIGS. 14A) as well as intracellular expression of CX3CR1 PU.1, IBA, P2RY12, TREM2 and TMEM119 by flow cytometry.

FIGS. 15A-C: Recovery of microglia post cryopreservation using manual vs control rate freezer. HPCs were placed in media to initiate microglia differentiation in the presence of MDM. The cells were cryopreserved at day 20 (FIG. 15B), day 23 (FIG. 15B), and day 26 (FIG. 15C) of differentiation using manual freezing protocol or a control rate freezer (CRF). The cryopreserved cells were transferred to liquid nitrogen for a week. Cryopreserved Microglia were thawed and placed in microglia maturation medium (MMM). The cultures were fed every 48 hrs with fresh maturation media. The cells were harvested on day 3, 5, 7, 10, 12 and 14-days post thaw and the recovery of viable cells with respect to the initial plating number was quantified.

FIG. 16: Efficiency of converting HPCs to microglia. Cryopreserved HPCs were differentiated to microglia in the presence of MDM (N=4). The total viable number of input HPCs and output microglia was quantified. The process efficiency was calculated based on the purity and absolute number of TREM2 positive cells present on day 23 of microglia differentiation divided by the absolute number of input viable HPCs.

FIGS. 17A-17C: Purity analysis of microglia cryopreserved either manually or in the presence of a control rate freezer on day 20 (FIG. 17A), day 23 (FIG. 17B) and day 26 (FIG. 17C) at thaw, 3 days post thaw and 10 days post thaw. Microglia cryopreserved on day 20, 23 and 26 were thawed and plated for 3, 5, 7, 10 and 12 days in Microglia Maturation Medium. The cells were stained for the presence of Pu1, IBA, CX3CR and P2RY12 expression by flow cytometry.

FIGS. 18A-18B: Viable manual hemocytometer cell counts of microglia cryopreserved either manually or in the presence of a control rate freezer on day 0 (FIG. 18A), and day 3 (FIG. 18B), post thaw to set up the phagocytosis assays with S. aureus bioparticles.

FIG. 19: Functional Characterization of microglia cryopreserved on day 20, day 23 and day 26 of differentiation using the manual or control rate freezer. Cryopreserved Microglia were thawed and plated at 15,000 viable cells/well in a 96 well plate in the presence of 200 μl Microglia Maturation medium per well. The cells were treated with diluted 1 μg/well of opsonized or non-opsonized pHrodo Red BioParticles (Thermo Fisher #A10010, 2 mg per vial; stored at −20° C.). The plate was placed on the IncuCyte and images of the phagocytosis were taken at various time points up to 5 days post thaw. Cells cryopreserved via control rate freezer method exhibit more robust phagocytosis (due to higher cell viability). Manual cryopreservation method revealed decreased/right-shifted rate of phagocytosis across all conditions (due to lower cell viability).

FIG. 20: Functional Characterization of live day 14 microglia and microglia cryopreserved on day 20, day 23 and day 26 of differentiation using the manual or control rate freezer assessed via live imaging on the IncuCyte system. Cryopreserved microglia were thawed and plated in MMM for three days. The viable cell counts at the end of three days were determined as described in FIG. 18B. 15,000 viable cells were plated in a 96 well plate in the presence of 200 μl Microglia Maturation medium (MMM) per well. The cells were fed fresh 50 μl media of MMM every 48 hours. The cells were treated with diluted 1 μg/well of opsonized or non-opsonized pHrodo Red BioParticles (Thermo Fisher #A10010, 2 mg per vial; stored at −20° C.). The plate was placed on the IncuCyte and images of the phagocytosis were taken at various time points up to 5, 7- and 14-days post thaw. Manual cryopreservation method revealed decreased/right-shifted rate of phagocytosis across all conditions (due to lower cell viability).

FIG. 21: The phagocytic efficiency ratio was determined by dividing the number of phagocytic red cell count/total cell number from the post thaw sample.

FIG. 22: Functional characterization of cryopreserved microglia using pHrodo Amyloid beta. Cryopreserved day 23 microglia were thawed and plated at 15,000 viable cells/well in a 96 well plate in the presence of 200 μl Microglia Maturation medium per well. The cells were treated with pHrodo Amyloid beta. The control set contained cells with media without any pHrodo Amyloid beta. The plate was placed on the IncuCyte and images of the phagocytosis were taken at various time points up to 24 hours post thaw.

FIGS. 23A-23B: Miniaturizing the differentiation of HPCs to microglia in the absence of ECM and in a 96 well format amenable to screening applications. Schematic representation of the differentiation of HPCs to microglia (FIG. 23A) and the different charged surfaces used in the experiments. Ultra-low attachment (ULA), Tissue Culture (TC) and Non-tissue culture (Non-TC) vessels (FIG. 23B).

FIGS. 24A-24B: End stage purity analysis of day 23 microglia in the presence of various charged surfaces. Cryopreserved HPCs were plated at a density of 20,000-35,000 viable cells/cm² on a 96 well Primaria plate or Ultra-low attachment, tissue culture (TC) or non-tissue culture plates (Non-TC) in the presence of 200 μl microglia differentiation medium per well. The cells were fed every 48 hrs with 50 μl media per well of MDM for the next 23 days of differentiation. The cells were harvested with cold PBS on day 23 and the total viable cell number was quantified using an automated cell counter. The cells were stained for surface expression of CD11b, CD45, CD33, TREM2 and intracellular expression of TREM2, IBA, P2RY12 and TMEM119.

FIGS. 25A-25B: Cytokines and Chemokines released by cryopreserved microglia. Day 23 cryopreserved microglia were thawed into MDM medium and plated on to Primaria 96 well plate at 50,000 cells/well. Cells were plated for three days prior to starting stimulations with 100 ng/ml LPS and 50 ng/ml interferon gamma. Stimulations were performed in triplicate for 24 hours. Supernatant was spun down to remove cells and debris and immediately placed at −20° C. The supernatants were analyzed on a multiplex Luminex assay. Average of multiple WT batches (FIG. 25A), and WT, Homozygous and Heterozygous TREM2 Knockouts (KOs), MECP2 HM and A53T-SNCA engineered lines (FIG. 25B) are shown.

FIG. 26: Cryopreserved microglia derived from multiple batches of Homozygous and Heterozygous TREM2 Knockouts (KOs), MECP2 and A53T-SNCA engineered lines were stained for the presence of surface expression of CD11b, CD45, TREM2, as well as intracellular markers PU.1, IBA-1, CX3CR1, P2RY12 and TMEM119. The engineered iPSC lines revealed comparable expression of TREM2 by flow cytometry.

FIGS. 27A-27B: (FIG. 27A) Release of soluble TREM2 in cryopreserved Microglia derived from Wildtype (WT), Heterozygous (HT) and Homozygous (HO) TREM2 KO engineered iPSCs. Levels of soluble TREM2 (sTREM2) were quantified from conditioned media from WT and TREM2 Heterozygous and Homozygous KO mutants using a Simple Step ELISA (AbCam). WT and TREM2 KO microglia were thawed and plated at the same density in maturation media in a 96 well Primaria plate. The spent media was collected at Day 3 post thaw and Day 7 post thaw. The cultures were half fed with fresh maturation media on Day 3 and Day 5 post thaw. (FIG. 27B) Release of soluble TREM2 in cryopreserved microglia derived from Wildtype (WT), MECP2 HM and A53T-SNCA engineered iPSCs. Levels of soluble TREM2 (sTREM2) were quantified from conditioned media from WT, MECP2 HM and A53T-SNCA engineered lines using a Simple Step ELISA (AbCam). Microglia were thawed and plated at the same density in maturation media in a 96 well Primaria plate. The spent media was collected at Day 3 post thaw and Day 7 post thaw. The cultures were half fed with fresh maturation media on Day 3 and Day 5 post thaw.

FIG. 28: List of media formulations to study the survival kinetics of WT, HT and HO TREM2 KO engineered microglia WT, 1185 HT TREM2 KO, 1187 HO TREM2 KO. Microglia were placed at a density of 15,000 viable cells per well in a 96 well plate in 250 μl of the microglia base medium containing 32 different variations of cytokine formulations. The kinetics of cell survival was captured on the IncuCyte system. NucGreen Dead diluted to 2 drops/mL was added to all wells containing cells with various media compositions to capture the number of dead cells with time. The images were captured every 8 hours and the experiment continued for 72 hours without any intermittent feeds.

FIGS. 29A-29C: WT (FIG. 29A), 1185 HT TREM2 KO (FIG. 29B), 1187 HO TREM2 KO (FIG. 29C) Microglia cell survival kinetics.

FIGS. 30A-30C: WT (FIG. 30A), 1185 HT TREM2 KO (FIG. 30B), 1187 HO TREM2 KO (FIG. 30C) Microglia cell survival kinetics with two cytokines.

FIGS. 31A-31C: WT (FIG. 31A), 1185 HT TREM2 KO (FIG. 31B), 1187 HO TREM2 KO (FIG. 31C) Microglia cell survival kinetics with three cytokines.

FIGS. 32A-32C: WT (FIG. 32A), 1185 HT TREM2 KO (FIG. 32B), 1187 HO TREM2 KO (FIG. 32C) Microglia cell survival kinetics with four cytokines.

FIGS. 33A-33E: WT, 1185 HT TREM2 KO, 1187 HO TREM2 KO Microglia were placed at a density of 15,000 viable cells in a 96 well plate in 250 μl of Microglia base medium (FIG. 33A) or MMM (FIG. 33B), or Microglia base medium supplemented with IL-34 (FIG. 32C), or Microglia base medium supplemented with IL-34 (FIG. 33D), or Microglia base medium supplemented with MCSF (FIG. 33D) or base medium supplemented with only IL-34 (FIG. 33C) or MSCF or a combination of IL-34 and MCSF (FIG. 33E). The kinetics of cell survival was captured on the IncuCyte system. NucGreen Dead diluted to 2 drops/mL was added to all wells containing cells with various media compositions to capture the number of dead cells with time. The images were captured every 8 hours and the experiment continued for 7 days without any intermittent feeds. The intensity of the NucGreen Dead quantifies the dead cells in the cultures.

FIGS. 34A-34H: Functional Characterization of WT, 1185 HT TREM2 KO, 1187 HO TREM2 KO microglia, cryopreserved on day 23 with pHrodo Red labelled bacterial BioParticles and pHrodo Red Amyloid beta. WT, 1185 HT TREM2KO, 1187 HO TREM2 KO microglia were plated at a density of 15,000 viable cells/cm² in a 96 well plate in 250 μl of MMM (FIGS. 34A-B) or MDM base (AKA microglia base medium) supplemented only MSCF (FIGS. 34C-D) or IL-34 (FIGS. 34E-F) or a combination of IL-34 and MCSF (FIGS. 34G-H) for three days post thaw. The cells were treated with diluted 1 μg/well of opsonized or non-opsonized pHrodo Bioparticles (Thermo Fisher # A10010, 2 mg per vial; stored at −20C) (FIGS. 34 A, C, E, G) or pHrodo Amyloid beta (FIGS. 34B, D, F, H). The plate was placed on the IncuCyte and image of the phagocytosis were taken at various time points up to 30 hrs. WT as well as engineered microglia revealed phagocytic function post thaw. The kinetics and the efficiency of phagocytosis varied between the WT, 1185 HT TREM2 KO, 1187 HO TREM2 KO microglia.

FIG. 35: Purity of microglia cultures in a Simplified Maturation Medium. Post thaw purity of day 23 cryopreserved Wild Type (WT) microglia in the presence of MMM or microglia base medium supplemented with combinations of two critical (IL-34, MSCF) cytokines in the maturation media. The purity was quantified at day 3, 7 and day 14 post thaw by harvesting the cells and the purity of CD45, CD33, TREM2, CD11b, CX3CR1, P2RY12, TMEM119, IBA, was determined at the end of the differentiation process by harvesting the cells and staining the cells for cell surface and intra cellular staining of markers by flow cytometry. Cryopreserved microglia retain viability, purity in maturation media supplemented with MSCF and IL-34. This simplified media will be valuable for co culture applications of cryopreserved microglia with neurons and astrocytes for developing TREM a brain organoids.

FIGS. 36A-36: (FIG. 36A) Schematic depicting screening experiment with cryopreserved microglia. (FIG. 36B) Table of compounds tested in screen.

FIGS. 37A- 37D: Results of microglia screening experiment with GW501516 (FIG. 37A), Leucettine L41 (FIG. 37B), Piceatannol (FIG. 37C), and Azeliragon (FIG. 37D).

FIGS. 38A-38D: Results of microglia screening experiment with J147 (FIG. 38A), Dibutryl-cAMP (FIG. 38B), Isradipine (FIG. 38C), and Bexarotene (FIG. 38D).

FIGS. 39A-39E: Results of microglia screening experiment with SB-431542 (FIG. 39A), SP600125 (FIG. 39B), GW2580 (FIG. 39C), PP2 (FIG. 39D), and SB239063 (FIG. 39E)

FIGS. 40A-40D: Results of microglia screening experiment with GW501516 (FIG. 40A), Leucettine L41 (FIG. 40B), Piceatannol (FIG. 40C), and Azeliragon (FIG. 40D) with LPS stimulation.

FIGS. 41A-41E: Results of microglia screening experiment with J147 (FIG. 41A), Dibutryl-cAMP (FIG. 41B), Isradipine (FIG. 41C), Bexarotene (FIG. 41D), and SB-43152 (FIG. 41E) with LPS stimulation.

FIGS. 42A-42D: Results of microglia screening experiment with SP600125 (FIG. 41A), GW2580 (FIG. 42B), PP2 (FIG. 42C), and SB239063 (FIG. 42D) with LPS stimulation.

FIG. 43: Summary of microglia screening experiment results.

FIGS. 44A-44G: (FIG. 44A) Microglia with ATP/BzATP All Traces—Ratio. (FIG. 44B) Microglia with ATP/BzATP Sample Traces—Ratio. (FIG. 44C) Response to BzATP in microglia. (FIG. 44D) Differential Response to ADP in microglia. (FIG. 44E) Response with 100 μM BzATP in the presence of P2X7 antagonist AZ11645373. (FIG. 44F) Response with 100 μM BzATP in the presence of P₂X₇ antagonist A438079. (FIG. 44G) Dose dependent response to demonstrate functional ADP dependent response in microglia in the presence of AZD1283 (a potent antagonist of the P2Y₁₂ receptor).

FIGS. 45A-45B: Release of soluble TREM2 in cryopreserved microglia derived from ANH and Disease Associated Microglia were quantified from conditioned media collected on day 3 (FIG. 45A) or day 7 (FIG. 45B) post thaw using a Simple Step ELISA (AbCam). ANH and DAM associated microglia were thawed and plated at the same density in maturation media in a 96 well Primaria plate. The spent media was collected at Day 3 post thaw and Day 7 post thaw. The cultures were half fed with fresh maturation media on Day 3 and Day 5 post thaw.

FIGS. 46A-46J: Cytokines and Chemokines released by cryopreserved microglia derived from ANH and Disease Associated Microglia from a panel of iPSC donors. Day 23 Cryopreserved microglia were thawed into MDM medium and plated on to Primaria 96 well plate at 50,000 cells/well. Cells were plated for three days prior to starting stimulations with 100 ng/ml LPS or IL-4+dBu-cAMP. Stimulations were performed in triplicate for 24 hours. Supernatant was spun down to remove cells and debris and immediately placed at −20° C. The supernatants were analyzed on a multiplex Luminex assay. Upon stimulation with LPS, release of M1 analytes (FIG. 46A), M2 analytes (FIG. 46B), interleukins (FIG. 46C), chemokines (FIG. 46D), and other analytes (FIG. 46E). Upon stimulation with IL-4+dBu- cAMP, release of M1 analytes (FIG. 46F), M2 analytes (FIG. 46G), interleukins (FIG. 46H), chemokines (FIG. 46I), and other analytes (FIG. 46J).

FIGS. 47A-47J: Demonstration of phagocytic function of AHN and Disease associated microglia using pHrodo labeled Staphylococcus aureus Bioparticles and Amyloid Beta: Cryopreserved AHN and Disease associated microglia were plated at 5,000 cells per well of 384 well Poly-D-Lysine plate. S. aureus bioparticles were added to each well at 0.5 μg/mL, Amyloid Beta was added at 1 μM/well. Kinetics of phagocytosis of S. aureus bioparticles and Amyloid Beta was quantified using Total Red Object Integrated Intensity using the IncuCyte Live-Cell Analysis System. (FIG. 47A) (ANH, S. aureus), (FIG. 47B) (ANH, Amyloid Beta), (FIG. 47C) (R47H Vs ANH, S. aureus), (FIG. 47D) (R47H Vs ANH, Amyloid Beta), (FIG. 47E) (CD33 Vs ANH, S. aureus), (FIG. 47F) (CD33 Vs ANH, Amyloid Beta), (FIG. 47G) (ABCA7 vs ANH, S. aureus), (FIG. 47H) (ABCA7 vs ANH, Amyloid Beta), (FIG. 47I) (APOE isoforms Vs ANH, S. aureus), (FIG. 47J) (APOE isoforms Vs ANH, Amyloid Beta).

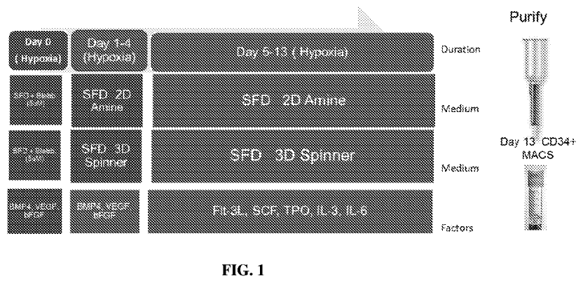

FIGS: 48A-48D: (FIG. 48A) A schematic description of the method to generate Neural Precursor Cells (NPCs) from iPSCs without using dual SMAD inhibition. The various steps involved, and the composition of medias used is described. (FIG. 48B) Summary of the kinetics of emergence of NPCs across three iPSC lines. The decrease in pluripotency markers and the emergence of NPCs specific markers on different days of the differentiation process. Quantification of purity performed by cell surface straining and intracellular staining by flowcytometry (FIG. 48C) Staining for Astrocytes derived from NPC across multiple passages in cultures. Purity of astrocytes quantified by cell surface and intracellular staining by flow cytometry. (FIG. 48D) Differentiation of NPCs to neurons and quantification of purity of end stage neurons by intracellular flow cytometry.

FIG. 49: Summary of surfaces compatible for derivation of iPSC derived cell lineages.

### DESCRIPTION OF ILLUSTRATIVE EMBODIMENTS

In certain embodiments, the present disclosure provides methods for the production of cells of multiple lineages, such as endothelial cells, mesenchymal stem cells (MSCs), and hematopoietic precursor cells (HPCs), from induced pluripotent stem cells (iPSCs). Generally, the method comprises differentiating iPSCs to the various lineage cells by using a charged surface. Specifically, the differentiation method may be in the absence of extracellular matrix (ECM) proteins and allows for the self-purification by passaging, such as for the production of MSCs and endothelial cells.

In further embodiments, methods are provided for the differentiation of endothelial cells to brain microvascular endothelial cells (BMECs) or lymphatic endothelial cells, the differentiation of MSCs to pericytes, and the differentiation of HPCs to microglia.

The process allows for the efficient generation of endothelial cells and MSCs without purification, such as MACS purification, or the use of ECM proteins. The process can be adapted to good manufacturing practice (GMP)-compliance.

Endothelial cells make up a network of interconnected cells in the human body that line blood vessels, lymphatic vessels, and form capillaries. Endothelial cells regulate the flow of nutrient substances and create and respond to diverse biologically active molecules and provide for potential uses in the tools space for screening compounds and drugs for vascular toxicity, vascular permeability, and therapeutic uses, including treatment of tissue ischemia and bioengineering of grafts. There have been many protocols used to derive endothelial cells. Almost all processes require a magnetic-activated cell sorting (MACs) separation using CD31 microbeads to generate a pure culture of endothelial cells.

In certain embodiments, the present disclosure provides methods to generate a pure population of endothelial cells from iPSCs, such as episomally reprogrammed iPSCs, by a two-step process. iPSC cells may be converted to hemogenic progenitor cells (HPCs) on a positively charged amine surface followed by further propagation and subsequent purification of endothelial cells in the presence of a negatively charged carboxyl surface. The endothelial cells may be derived by hemogenic endothelial cells without any MACs purification step. The cells express CD31/CD144/CD105 at high purity and can be propagated to retain purity and cryopreserved at earlier and late passages.

Mesenchymal stem cells (MSCs) isolated from adult human tissues are capable of proliferating in vitro and maintaining their multipotency, making them attractive cell sources for regenerative medicine. However, the availability and capability of self-renewal under current preparation regimes are limited. iPSCs now offer an alternative, similar cell source to MSCs. Accordingly, certain embodiments of the present disclosure provide methods for differentiating MSCs from iPSCs, such as episomally reprogrammed iPSCs, by initiating mesodermal differentiation on a positively charged amine surface using GMP compatible conditions. MSCs derived by this process express all purity markers for the MSC lineage as well as displaying self-renewal and multipotency. These cells can be scaled up for clinical applications.

In further embodiments, the present disclosure provides methods for the differentiation of MSCs into pericytes (PCs). Pericytes, also known as mural cells, ensheath blood microvessels (i.e., capillaries, arterioles, and venules) and are generally understood as having an organizational or structural role in angiogenesis. Multiple criteria including location, morphology, gene or protein expression patterns, and density around vessels are used to identify immature and mature pericytes. In general, a pericyte obtained according to a method provided herein can be identified based on expression of known pericyte molecular markers such as, without limitation, PDGFRβ, desmin (DES), CD13 (ANPEP; alanyl (membrane) aminopeptidase), a-SMA, RGSS (Regulator of G-protein Signaling 5), NG2 (also known as CSPG4; chondroitin sulfate proteoglycan 4), CD248 (endosialin), ANG-1, CD146, CD44, CD90, and CD13.

Microglia are innate immune cells of the central nervous system that perform critical roles in brain development, homeostasis, and immune regulation. They are hard to acquire from human fetal and primary tissues. Thus, further embodiments of the present disclosure provide methods for the generation, characterization and cryopreservation of human iPSC-derived microglia (iMGL) from HPCs, such as episomally reprogrammed iCell HPCs, under defined conditions. Cryopreserved iMGL retain purity, secrete immunomodulatory cytokines and phagocytose pHrodo Red labelled bacterial BioParticles and Amyloid βeta aggregates. The ability to produce essentially limitless quantities of iMGLs holds great promise for accelerating human neuroscience search into the role of microglia in normal and diseased states.

Further provided herein are microglia with disruption in TREM2, MeCP2, and/or SCNA. These microglia derived from patient derived iPSC provide an in vitro tool to create a more accurate model to understand complex interactions between human microglia, neurons, astrocytes in a 2D or 3D organoid systems and mimic neurogenerative diseases.

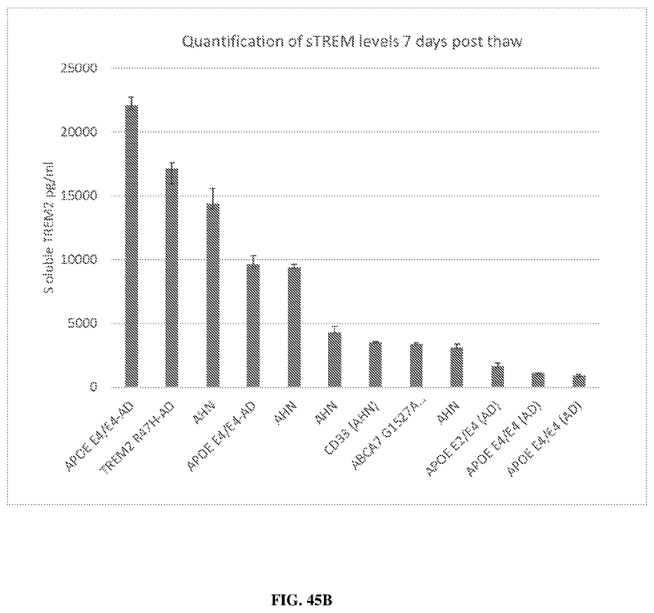

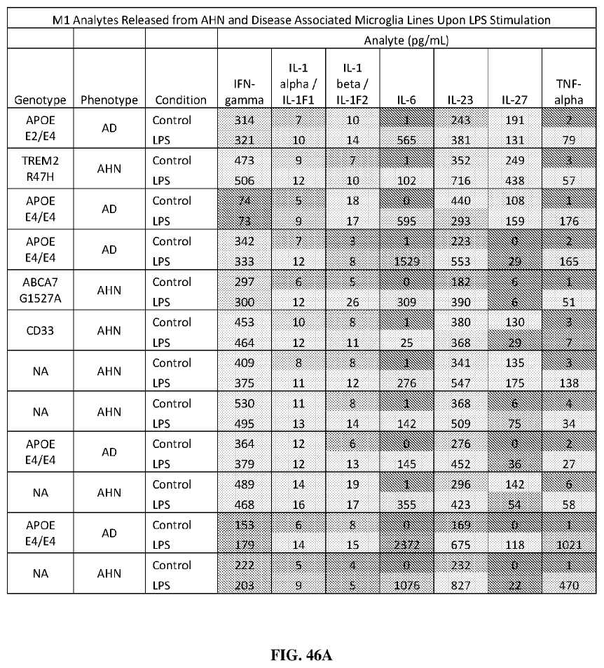

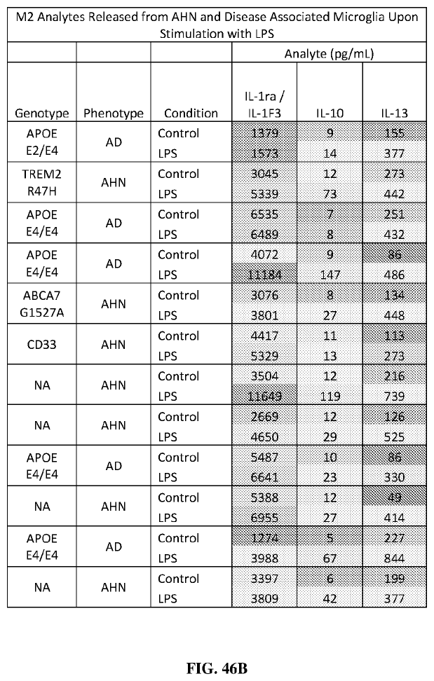

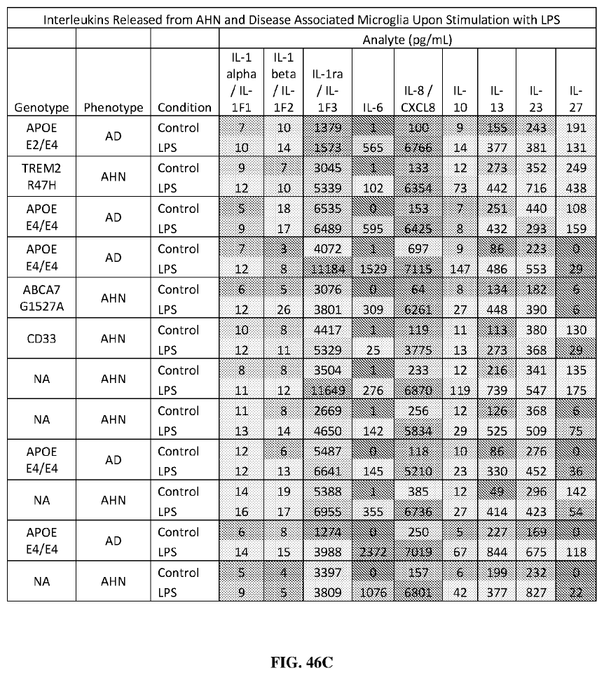

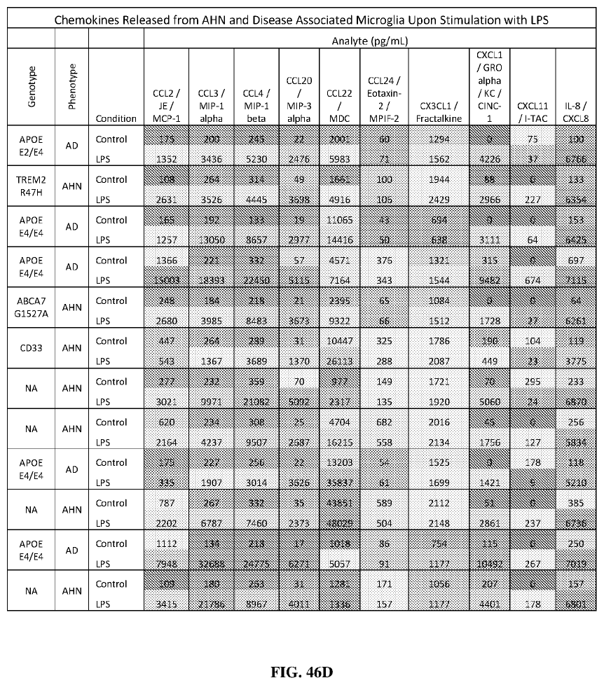

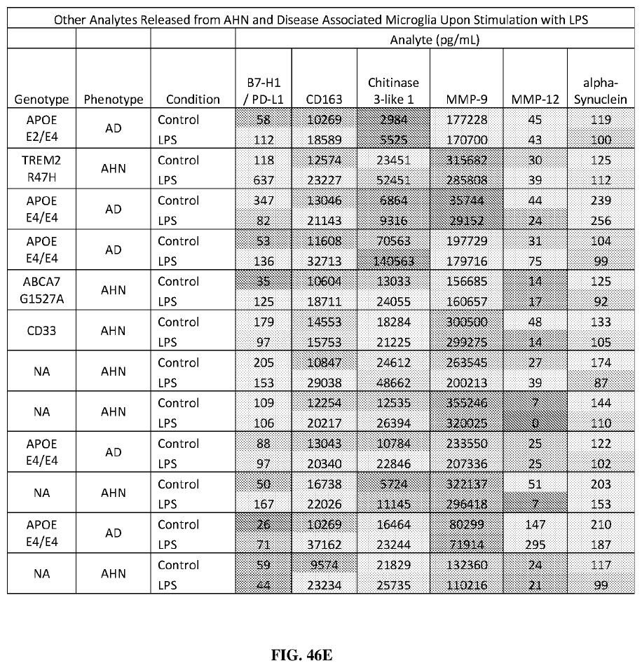

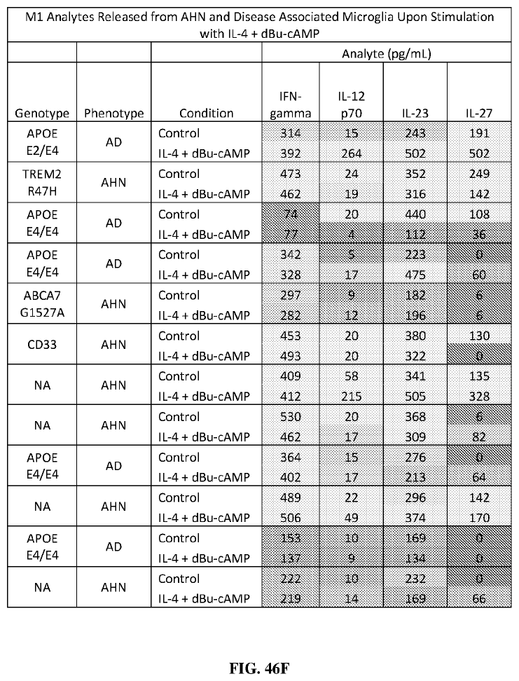

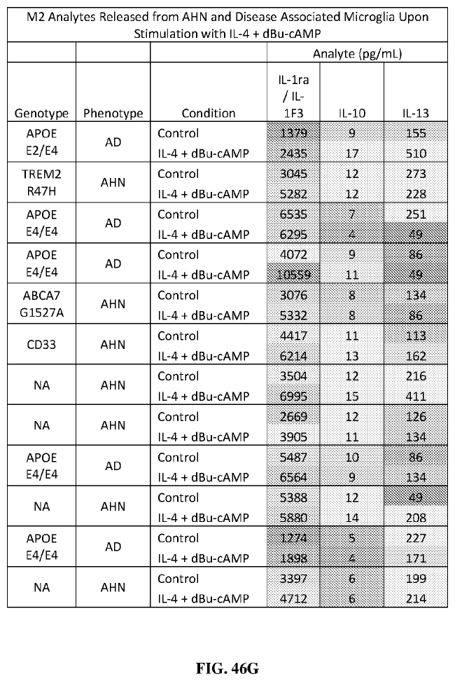

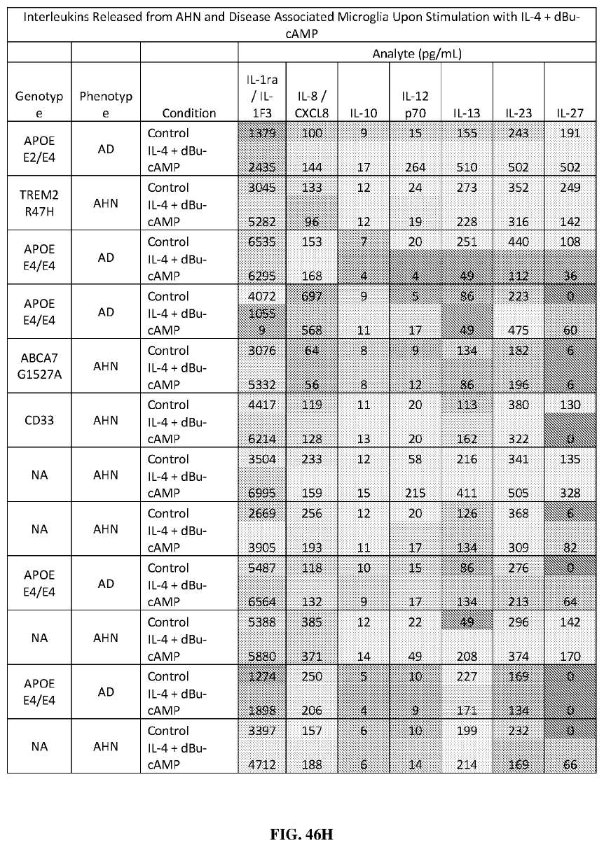

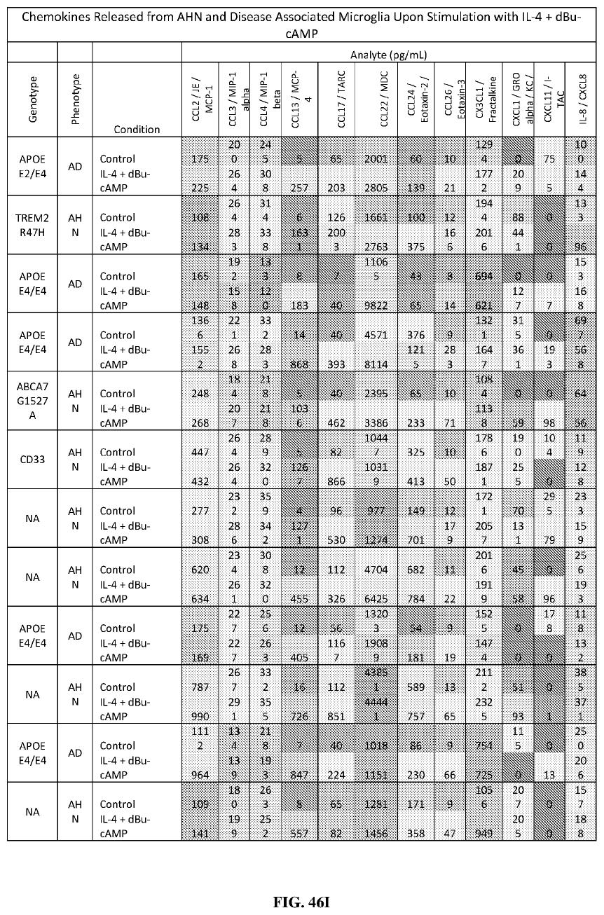

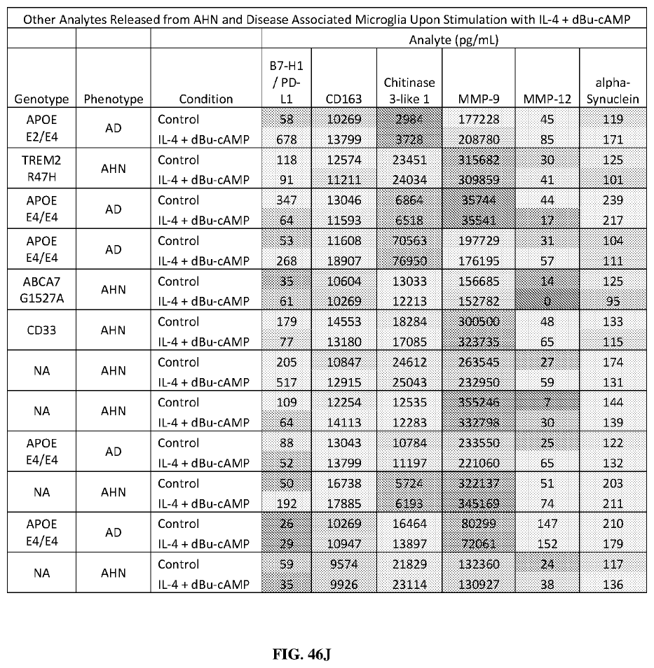

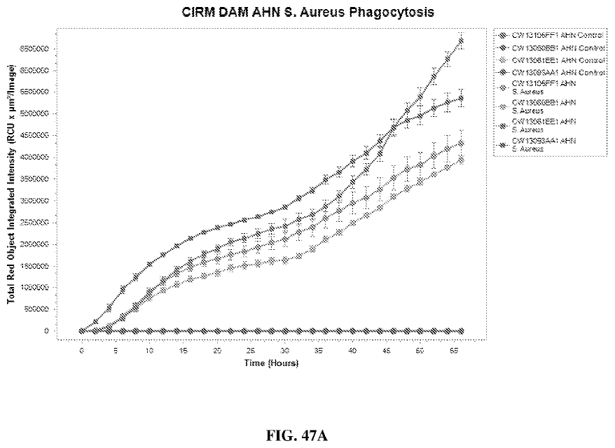

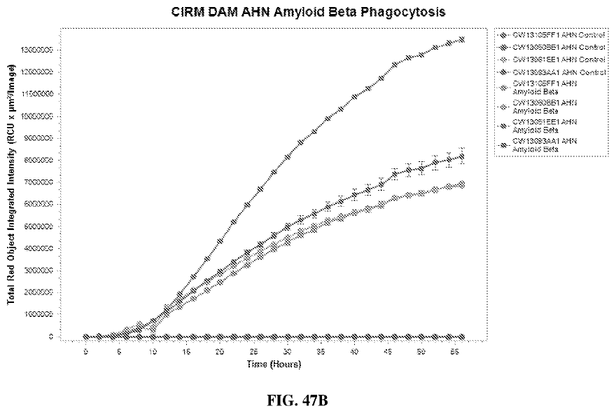

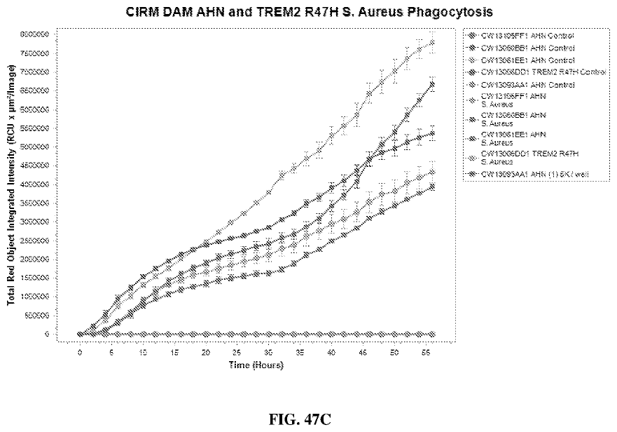

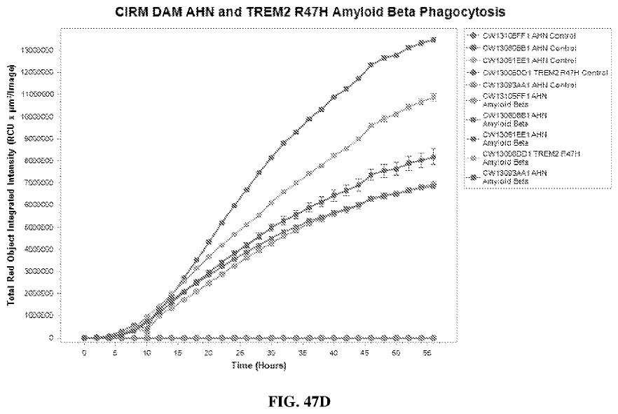

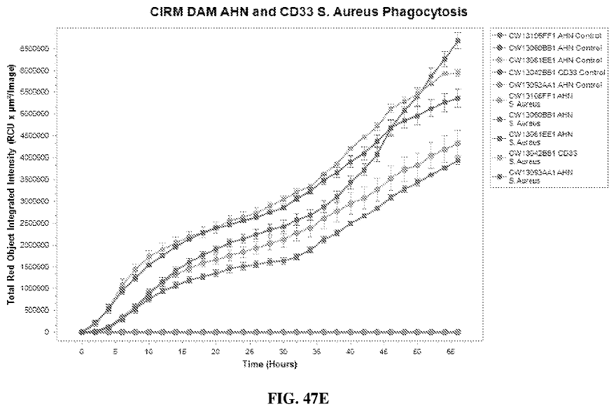

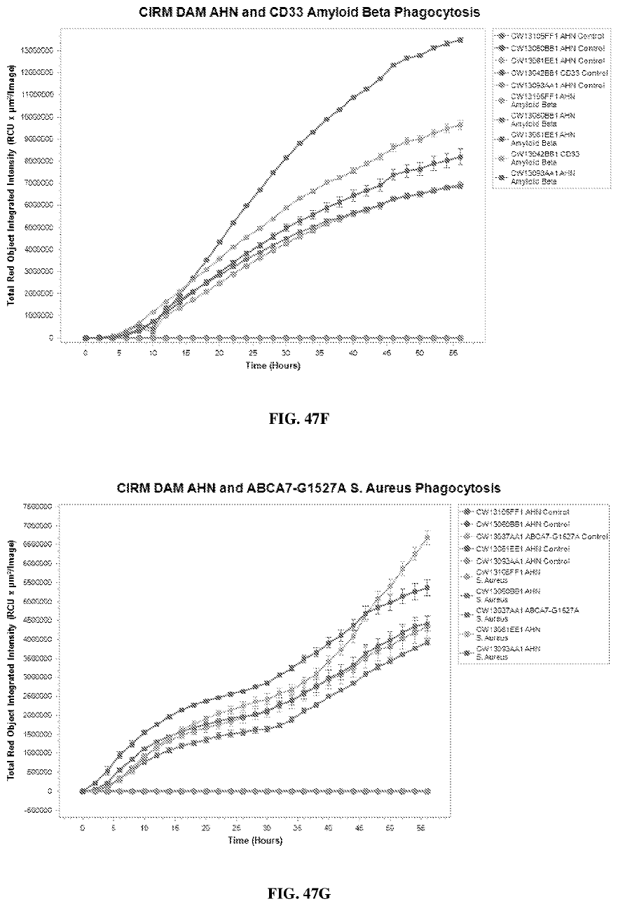

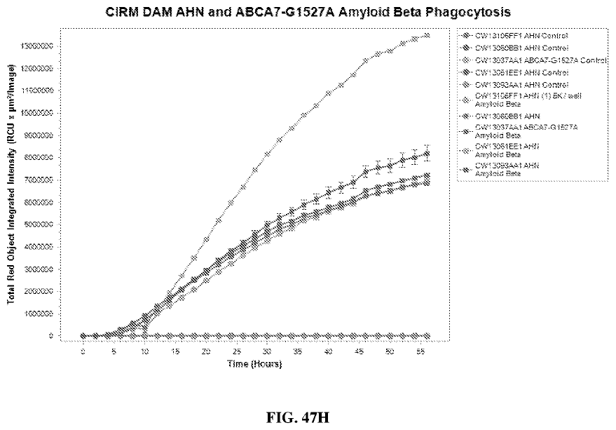

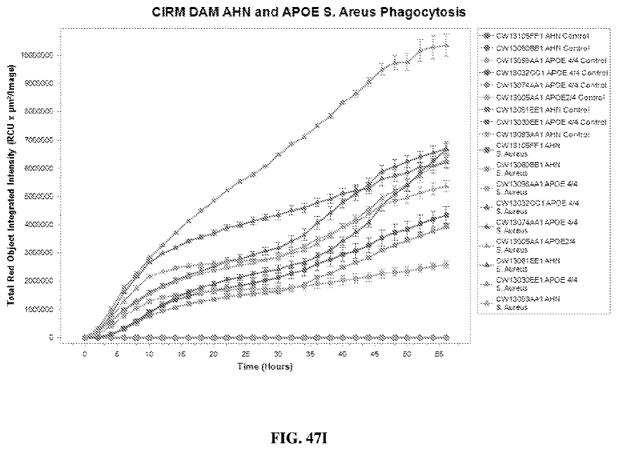

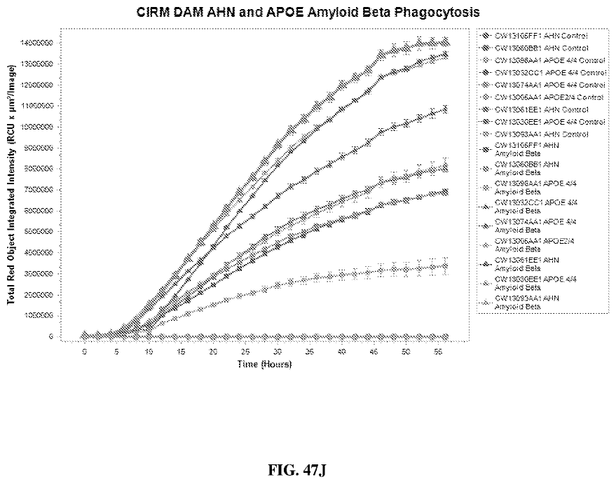

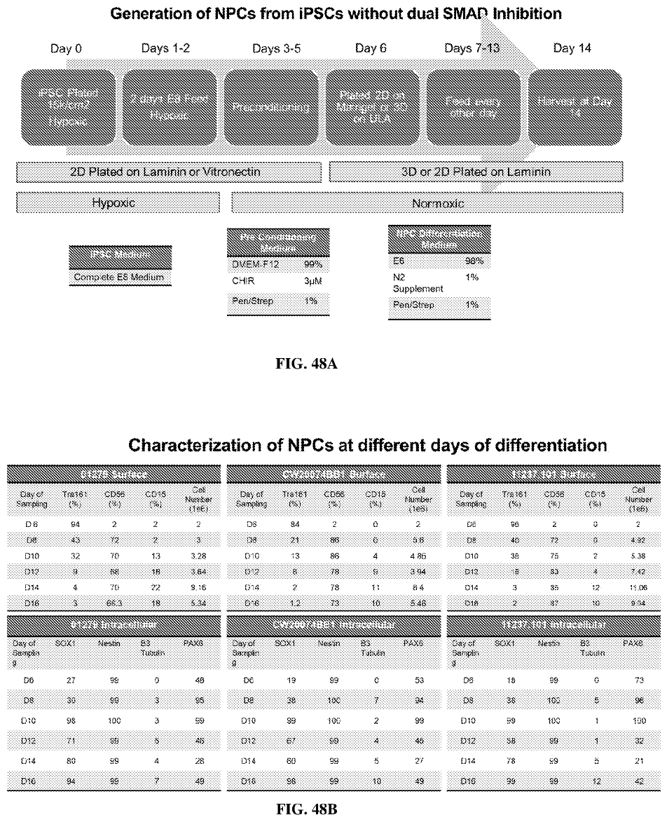

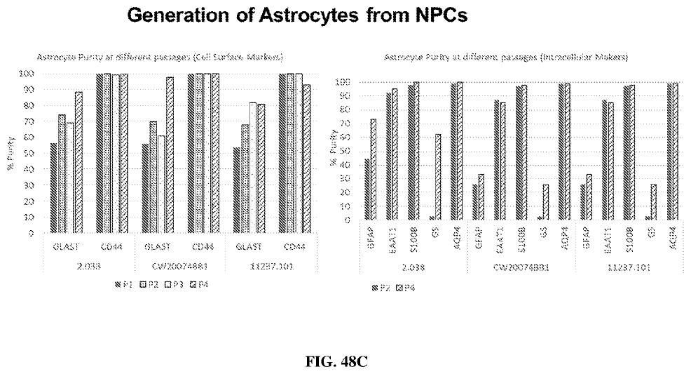

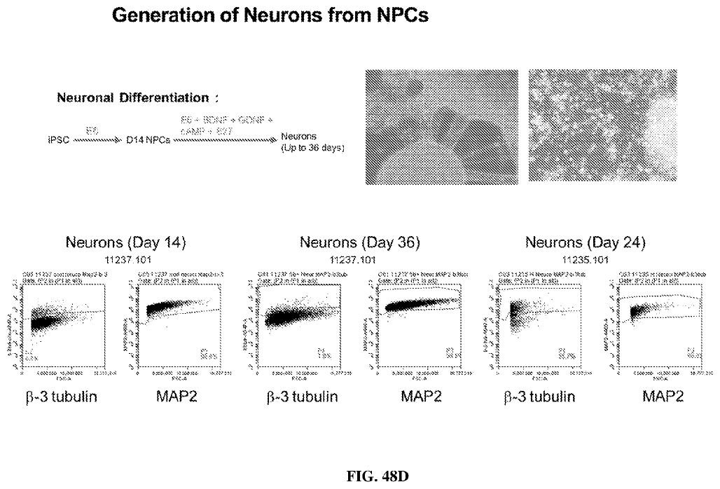

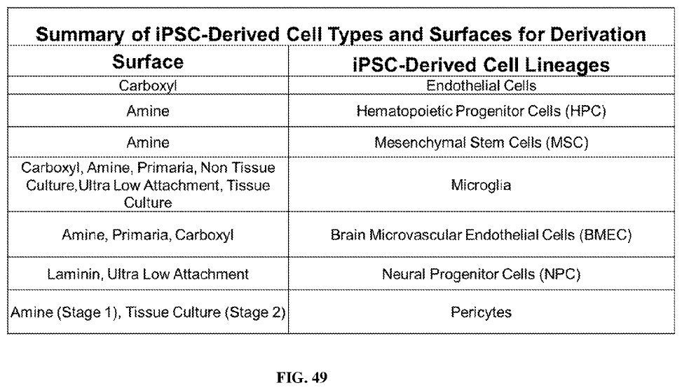

Also provided herein are methods of differentiating iPSCs to neural precursor cells (NPCs) without inhibition of SMAD signaling. These NPCs can be co-cultured with iPSC derived microglia to generate long term co-culture assays to mimic human brain development and complex intercellular interactions between neural lineages, microglia, endothelial cells, pericytes, and astrocytes in a dish derived from normal and/or disease-specific iPSC cells. The iPSCs may be maintained under hypoxic conditions before the onset of differentiation to generate NPCs. To initiate neural precursor differentiation, iPSCs may be seeded in the presence of a ROCK inhibitor or blebbistatin on an ECM-coated surface. The cells can be placed in media for the next 48 hours in the absence of a ROCK inhibitor. Next, the cells are pre-conditioned in DMEMF12 media supplemented with a GSK3 inhibitor for 72hours with a daily change in media under normoxic conditions. Cells can be harvested at the end of the preconditioning step and either replated back in a 2D format on ECM-coated plates or as 3D aggregates using Ultra low attachment plates or spinner flasks in the presence of a ROCK inhibitor or blebbistatin. The cultures may be fed every other day with E6 media supplemented with N2 for the next 8 days under normoxic conditions of produce NPCs. The different steps involved in the generation of NPCs are outlined in FIG. 45A.

The cells produced by the present methods may be used for disease modeling, drug discovery, and regenerate medicine. Also provided herein are methods of using the present cells (e.g., MSCs, endothelial cells, neural precursor cells and pericytes) for the production of a brain organoid or blood-brain barrier (BBB) model.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains ≤30%, <20%, ≤15%, more preferably ≤10%, even more preferably ≤5%, or most preferably ≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco' s Modified Eagle' s Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Treatment” or “ treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

The term “extracellular matrix protein” refers to a molecule which provides structural and biochemical support to the surrounding cells. The extracellular matrix protein can be recombinant and also refers to fragments or peptides thereof. Examples include collagen and heparin sulfate.

A “three-dimensional (3-D) culture” refers to an artificially-created environment in which biological cells are permitted to grow or interact with their surroundings in all three dimensions. The 3-D culture can be grown in various cell culture containers such as bioreactors, small capsules in which cells can grow into spheroids, or non-adherent culture plates. In particular aspects, the 3-D culture is scaffold-free. In contrast, a “two-dimensional (2-D)” culture refers to a cell culture such as a monolayer on an adherent surface.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

### II. iPSC Differentiation Methods

### A. HPCs

The iPSCs can be differentiated into HPCs by methods known in the art such as described in U.S. Pat. No. 8,372,642, which is incorporated by reference herein. In one method, combinations of BMP4, VEGF, Flt3 ligand, IL-3, and GM-CSF may be used to promote hematopoietic differentiation. In certain embodiments, the sequential exposure of cell cultures to a first media to prepare iPSCs for differentiation, a second media that includes BMP4, VEGF, and FGF, followed by culture in a third media that includes Flt3 ligand, SCF, TP0, IL-3, and IL-6 can differentiate pluripotent cells into HPCs and hematopoietic cells. The second defined media can also comprise heparin. Further, inclusion of FGF-2 (50 ng/ml) in the media containing BMP4 and VEGF can enhance the efficiency of the generation of hematopoietic precursor cells from pluripotent cells. In addition, inclusion of a Glycogen synthase kinase 3 (GSK3) inhibitor (e.g., CHIR99021, BIO, and SB-216763) in the first defined media can further enhance the production of HPCs.

Generally, differentiation of pluripotent cells into hematopoietic precursor cells may be performed using defined or undefined conditions. It will be appreciated that defined conditions are generally preferable in embodiments where the resulting cells are intended to be administered to a human subject. Hematopoietic stem cells may be derived from pluripotent stem cells under defined conditions (e.g., using a TeSR media), and hematopoietic cells may be generated from embryoid bodies derived from pluripotent cells. In other embodiments, pluripotent cells may be co-cultured on OP9 cells or mouse embryonic fibroblast cells and subsequently differentiated.

Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins. Three-dimensional EBs can thus be used to produce some fraction of hematopoietic cells and endothelial cells.

To promote aggregate formation, the cells may be transferred to low-attachment plates for an overnight incubation in serum-free differentiation (SFD) medium, consisting of 75% IMDM (Gibco), 25% Ham's Modified F12 (Cellgro) supplemented with 0.05% N2 and 1% B-27 without RA supplements, 200 mM 1-glutamine, 0.05 mg/ml Ascorbic Acid-2-phosphate Magnesium Salt (Asc 2-P) (WAKO), and 4.5×10⁻⁴ MTG. The next day the cells may be collected from each well and centrifuged. The cells may then be resuspended in “EB differentiation media,” which consists of SFD basal media supplemented with about 50 ng/ml bone morphogenetic factor (BMP4), about 50 ng/ml vascular endothelial growth factor (VEGF), and 50 ng/ml zb FGF for the first four days of differentiation. The cells are half fed every 48 hrs. On the fifth day of differentiation the media is replaced with a second media comprised of SFD media supplemented with 50 ng/ml stem cell factor (SCF), about 50 ng/ml Flt-3 ligand (Flt-3L), 50 ng/ml interleukin-6 (IL-6), 50 ng/ml interleukin-3 (IL-3), 50 ng/ml thrombopoieitin (TPO). The cells are half fed every 48 hrs with fresh differentiation media. The media changes are performed by spinning down the differentiation cultures at 300 g for 5 minutes and aspirating half the volume from the differentiating cultures and replenishing it with fresh media. In certain embodiments, the EB differentiation media may include about BMP4 (e.g., about 50 ng/ml), VEGF (e.g., about 50 ng/ml), and optionally FGF-2 (e.g., about 25-75 ng/ml or about 50 ng/ml). The supernatant may be aspirated and replaced with fresh differentiation medium. Alternately the cells may be half fed every two days with fresh media. The cells may be harvested at different time points during the differentiation process.

HPCs may be cultured from pluripotent stem cells using a defined medium. Methods for the differentiation of pluripotent cells into hematopoietic CD34⁺stem cells using a defined media are described, e.g., in U.S. application Ser. No. 12/715,136 which is incorporated by reference in its entirety. It is anticipated that these methods may be used with the present disclosure.

For example, a defined medium may be used to induce hematopoietic CD34⁺ differentiation. The defined medium may contain the growth factors BMP4, VEGF, Flt3 ligand, IL-3 and/or GMCSF. Pluripotent cells may be cultured in a first defined media comprising BMP4, VEGF, and optionally FGF-2, followed by culture in a second media comprising either (Flt3 ligand, IL-3, and GMCSF) or (Flt3 ligand, IL-3, IL-6, and TPO). The first and second media may also comprise one or more of SCF, IL-6, G-CSF, EPO, FGF-2, and/or TPO. Substantially hypoxic conditions (e.g., less than 20% O2) may further promote hematopoietic or endothelial differentiation.

Cells may be substantially individualized via mechanical or enzymatic means (e.g., using a trypsin or TrypLE™). A ROCK inhibitor (e.g., H1152 or Y-27632) may also be included in the media. It is anticipated that these approaches may be automated using, e.g., robotic automation.

In certain embodiments, substantially hypoxic conditions may be used to promote differentiation of pluripotent cells into hematopoietic progenitor cells. As would be recognized by one of skill in the art, an atmospheric oxygen content of less than about 20.8% would be considered hypoxic. Human cells in culture can grow in atmospheric conditions having reduced oxygen content as compared to ambient air. This relative hypoxia may be achieved by decreasing the atmospheric oxygen exposed to the culture media. Embryonic cells typically develop in vivo under reduced oxygen conditions, generally between about 1% and about 6% atmospheric oxygen, with carbon dioxide at ambient levels. Without wishing to be bound by theory, it is anticipated that hypoxic conditions may mimic an aspect of certain embryonic developmental conditions. As shown in the below examples, hypoxic conditions can be used in certain embodiments to promote additional differentiation of induced pluripotent cells into a more differentiated cell type, such as HPCs.

The following hypoxic conditions may be used to promote differentiation of pluripotent cells into hematopoietic progenitor cells. In certain embodiments, an atmospheric oxygen content of less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, about 5%, about 4%, about 3%, about 2%, or about 1% may be used to promote differentiation into hematopoietic precursor cells. In certain embodiments, the hypoxic atmosphere comprises about 5% oxygen gas.

Regardless of the specific medium being used in any given hematopoietic progenitor cell expansion, the medium used is preferably supplemented with at least one cytokine at a concentration from about 0.1 ng/mL to about 500 ng mL, more usually 10 ng/mL to 100 ng/mL. Suitable cytokines include but are not limited to, c-kit ligand (KL) (also called steel factor (StI), mast cell growth factor (MGF), and stem cell factor (SCF)), IL-6, G-CSF, IL-3, GM-CSF, IL-1α, IL-11 MIP-1α, LIF, c-mpl ligand/TPO, and flk2/flk3 ligand (F1t2L or Flt3L). Particularly, the culture will include at least one of SCF, Flt3L and TPO. More particularly, the culture will include SCF, Flt3L and TPO.

In one embodiment, the cytokines are contained in the media and replenished by media perfusion. Alternatively, when using a bioreactor system, the cytokines may be added separately, without media perfusion, as a concentrated solution through separate inlet ports. When cytokines are added without perfusion, they will typically be added as a 10× to 100× solution in an amount equal to one-tenth to 1/100 of the volume in the bioreactors with fresh cytokines being added approximately every 2 to 4 days. Further, fresh concentrated cytokines also can be added separately in addition, to cytokines in the perfused media.

### Exemplary HPC Differentiation Method

2D HPC differentiation: iPSCs may be maintained on MATRIGEL™ or Vitronectin in the presence of E8 and adapted to hypoxia for at least 5-10 passages. Cells are split from sub confluent iPSCs and plated at a density of 0.25 million cells/well onto Amine culture dishes in the presence Serum Free Defined (SFD) media supplemented with 5 uM blebbistatin. 24 hrs post plating SFD media supplemented with 50 ng/ml of BMP4, VEGF and FGF2 is added to the culture. The following day, fresh media is exchanged to remove blebbistatin. On the fifth day of the differentiation process, the cells are placed in media containing 50 ng/ml Flt-3 Ligand, SCF, TP0, IL3 and IL6 with 5U/m1 of heparin. The cells are fed every 48 hrs throughout the differentiation process. The entire process is performed under hypoxic conditions and on charged amine plates. HPCs are quantified by the presence of CD43/CD34 cells and CFU.

3D HPC Differentiation: Cells were split from sub confluent iPSCs and plated at a density of 0.25-0.5 million cells per ml into a spinner flask in the presence of Serum Free Defined (SFD) media supplemented with 5 μM blebbistatin or 1 μM H1152. 24 hrs post plating SFD media supplemented with 50 ng/ml of BMP4, VEGF and FGF2 was exchanged. On the fifth day of the differentiation process the cells were placed in media containing 50 ng/ml Flt-3 Ligand, SCF, TP0, IL3 and IL6 with 5-10 U/ml of heparin. The cells were fed every 48 hrs throughout the differentiation process. The entire process was performed under hypoxic conditions. HPCs quantified by presence of CD43/CD34. HPCs are MACS sorted using CD34 beads.

### B. Gene Disruption

In certain aspects, TREM2, MeCP2, and/or SCNA gene expression, activity or function is disrupted in cells, such as PSCs (e.g., ESCs or iPSCs). In some embodiments, the gene disruption is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the disruption can be effected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the disruption of the expression, activity, and/or function of the gene is carried out by disrupting the gene. In some aspects, the gene is disrupted so that its expression is reduced by at least at or about 20, 30, or 40%, generally at least at or about 50, 60, 70, 80, 90, or 95% as compared to the expression in the absence of the gene disruption or in the absence of the components introduced to effect the disruption.

In some embodiments, the disruption is transient or reversible, such that expression of the gene is restored at a later time. In other embodiments, the disruption is not reversible or transient, e.g., is permanent.

In some embodiments, gene disruption is carried out by induction of one or more double-stranded breaks and/or one or more single-stranded breaks in the gene, typically in a targeted manner In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene disruption is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct. In particular aspects, the siRNA suppresses both wild-type and mutant protein translation from endogenous mRNA.

In some embodiments, the disruption is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 2005/0064474; 2006/0188987 and 2007/0218528, incorporated by reference in their entireties herein, for details regarding fusions of DNA-binding domains and nuclease cleavage domains. In some aspects, the additional domain is a nuclease domain Thus, in some embodiments, gene disruption is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

### 1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some aspects, disruption of MeCP2 is carried out by contacting a first target site in the gene with a first ZFP, thereby disrupting the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains.

In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

In some embodiments, ZFNs target a gene present in the engineered cell. In some aspects, the ZFNs efficiently generate a double strand break (DSB), for example at a predetermined site in the coding region of the gene. Typical regions targeted include exons, regions encoding N terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

### 2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. See, U.S. Patent Publication No. 2011/0301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, 1998) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. A library of TALENs targeting 18,740 human protein-coding genes has been constructed. Custom-designed TALE arrays are commercially available through Cellectis Bioresearch (Paris, France), Transposagen Biopharmaceuticals (Lexington, Ky., USA), and Life Technologies (Grand Island, N.Y., USA).

In some embodiments the TALENs are introduced as trans genes encoded by one or more plasmid vectors. In some aspects, the plasmid vector can contain a selection marker which provides for identification and/or selection of cells which received said vector.

### 3. RGENs (CRISPR/Cas Systems)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

### C. Charged Cell Surfaces

In some embodiments, the present disclosure concerns charged surfaces for cell culture. The charged surface may be positively charged, such as an amine surface or nitrogen-containing functional groups, or negatively charged, such as a carboxyl surface or oxygen-containing functional groups. The cell surfaces may be treated to alter the surface charge of the culture vessel.

In some aspects, the surface is neutrally charged, such as a surface comprising both negatively charged and positively charged functional groups. For example, the CORNING PRIMARIA® surface features a unique mixture of oxygen-containing (negatively charged) and nitrogen-containing (positive charged) functional groups on the polystyrene surface. The surface supports the growth of cells that can exhibit poor attachment or limited differentiation potential when cultured on traditional TC surfaces. In some aspects, the surface comprises a ULA surface coating. For example, the corning ultra-low attachment surface is a covalently bound hydrogel layer that is hydrophilic and neutrally charged. Since proteins and other biomolecules passively adsorb to polystyrene surfaces through either hydrophobic or ionic interactions, this hydrogel naturally inhibits nonspecific immobilization via these forces, thus inhibiting subsequent cell attachment. This surface is very stable, noncytotoxic, biologically inert and nondegradable. Other examples that could support the generation of microglia from HPCs include: Corning CellBIND culture (U.S. Pat. No. 6,617,152) uses a higher energy microwave plasma to incorporate more oxygen onto the polystyrene surface rendering it more hydrophilic (wettable) while increasing the stability of the surface compared with traditional plasma or corona discharge treated surfaces. Corning Synthemax self-coating substrate is a unique, animal-free, synthetic Vitronectin-based peptide containing the RGD motif and flanking sequences. The synthetic peptides are covalently bound to a polymer backbone for passive coating, orienting, and presenting the peptide for optimal cell binding and signaling.

The cell culture surface may be coated with a plasma polymerized film. The source of the plasma polymerization is one or more monomers. Useful polymerizable monomers may include unsaturated organic compounds such as olefinic amines, halogenated olefins, olefinic carboxylic acids and carboxylates, olefinic nitrile compounds, oxygenated olefins and olefinic hydrocarbons. In some embodiments, the olefins may include vinylic and allylic forms. In other embodiments, cyclic compounds such as cyclohexane, cyclopentane and cyclopropane may be used.

As will be recognized by those skilled in the art, various plasma polymerization techniques may be utilized to deposit the one or more monomers onto the cell culture surfaces. Preferably, a positively charged polymerized film is deposited on the surfaces.

As will be appreciated by one skilled in the art, the plasma polymerized surface may have a negative charge depending on the proteins to be used therewith. Amine is preferably used as the monomer source of the polymer. In some embodiments, the plasma polymerized monomer is made using plasma sources to generate a gas discharge that provides energy to initiate polymerization of gaseous monomers and allows a thin polymer film to deposit on a culture vessel. Cyclic compounds may be utilized which may include gas plasmas by glow discharge methods. Derivatives of these cyclic compounds, such as 1,2-diaminocyclohexane for instance, are also commonly polymerizable in gas plasmas.

Mixtures of polymerizable monomers may be used. Additionally, polymerizable monomers may be blended with other gases not generally considered as polymerizable in themselves, examples being argon, nitrogen and hydrogen.

It is contemplated that any culture vessel that is useful for adherent cultures may be used. Preferred cell culture vessel configurations contemplated by the present disclosure include multiwell plates (such as 6-well, 12-well and 24-well plates), dishes (such as petri dishes), test tubes, culture flasks, roller bottles, tube or shaker flasks, and the like.

Material for the cell culture surface may include plastic (e.g. polystyrene, acrylonitrile butadiene styrene, polycarbonate); glass; microporous filters (e.g., cellulose, nylon, glass fiber, polyester, and polycarbonate); materials for bio-reactors used in batch or continuous cell culture or in genetic engineering (e.g., bioreactors), which may include hollow fiber tubes or micro carrier beads; polytetrafluoroethylene (Teflon®), ceramics and related polymeric materials.

In particular aspects, the cell culture is free of or essentially free of any extracellular matrix proteins, such as laminin, fibronectin, vitronectin, MATRIGEL™, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin.

### D. Differentiation of HPCs to Microglia

Microglia are innate immune cells of the central nervous system that perform critical roles in brain development, homeostasis, and immune regulation. They are hard to acquire from human fetal and primary tissues. In certain embodiments, the present methods describe the generation, characterization and cryopreservation of human iPSC-derived microglia (iMGL) from episomally reprogrammed HPCs under defined conditions. Cryopreserved iMGL retain purity, secrete immunomodulatory cytokines and phagocytose pHrodo Red labelled bacterial BioParticles and Amyloid βeta aggregates. The ability to produce essentially limitless quantities of iMGLs holds great promise for accelerating human neuroscience research into the role of microglia in normal and diseased states.

In an exemplary method, fresh or cryopreserved HPCs are thawed and plated in Microglia Differentiation Media comprising FLT-3 ligand and IL-3. The cells may be plated at a density of 10-50 K/cm2, such as 20-35K/cm2. The Microglia Differentiation Medium may comprise IL-34, TGFβ1, or M-CSF (MDM). The culturing may be performed on MATRIGEL™ coated plate or a charged surface such as a Primaria plate or Ultra low attachment plate or a tissue culture plate (TC) or a non-tissue culture plate (Non-TC) and may be high-throughput, such as a 96 well plate (e.g., 200 μl Microglia Differentiation Medium per well). The cells may be half fed every 48 hrs with 50 μl media per well of 2× Microglia Differentiation media (MDM) the next 23 days of differentiation. In specific aspects, the differentiation is performed in the absence of ECM proteins, such as MATRIGEL®. The cells are harvested with cold PBS on day 23 and the total viable cell number is quantified using an automated cell counter. The cells are stained for surface expression of CD11b, CD11c, CD45, CD33, TREM-2 and intracellular expression of TREM-2, IBA, CX3CR1, P2RY12 and TMEM119.

### E. Endothelial Cells

iPSCs maintained on MATRIGEL™ or Vitronectin in the presence of E8, may be adapted to hypoxia for at least 5-10 passages. Cells may be split from sub confluent iPSCs and plated at a density of 0.25 million cells/well onto Amine culture dishes in the presence Serum Free Defined (SFD) media supplemented with 5 uM blebbistatin or 1 uM

H1152. 24 hrs post plating SFD media supplemented with 50 ng/ml of BMP4, VEGF and FGF2 may be added to the culture. The cells may be fed every 48 hrs throughout the differentiation process. The entire process may be performed under hypoxic conditions. The cells harvested at the end of differentiation can be cryopreserved or replated on a carboxyl surface at a density of 25k/cm2 to initiate endothelial differentiation in the presence of VascuLife VEGF Endothelial Medium or SFD Endothelial Medium.

In an exemplary method, cryopreserved day 6 HPCs or live cultures are plated at 25k/cm² on a carboxyl surface in the presence of VascuLife VEGF Endothelial Medium or SFD Endothelial Medium and hypoxic conditions. The cells are given a fresh feed of endothelial medium 24 hrs post plating and the cultures are fed every 48 hours until they reached confluency. It may take 5-6 days for cells to reach confluency. The cells are harvested using TrypLE Select, stained for surface endothelial markers CD31, CD105 and CD144 and replated onto a carboxyl surface at 25k/cm² with endothelial medium and placed in hypoxic incubator conditions. The cells are given a full feed of endothelial medium on days 2, 4, 6 post-split. On day 7, the cells are harvested, stained, and replated in the same manner three more times.

In some embodiments, the endothelial cells are converted to Brain Microvascular Endothelial cells. In an exemplary method, live or cryopreserved HPCs (e.g., day 7 HPCs derived on Amine surface in the presence of SFD supplemented with BMP4, VEGF and FGF2) are plated onto a ECM containing fibronectin (e.g., 50-200 μg/mL, particularly 100 μg/mL) and Collagen I (e.g., 100-500 μg/mL, particularly 400 μg/mL) with ECRA Medium (Human Endothelial SFM [Gibco], 1% Platelet-poor plasma-derived bovine serum [Fisher], 20 ng/mL bFGF [Promega], 10 uM Retinoic Acid). The cells may be plated at a density of 50-100 k/cm², particularly 75 k/cm². The cultures can be maintained under hypoxic incubator conditions. The cultures may be fed with ECRA Medium every other day until confluent. Confluent cultures are then harvested, such as by using TrypLE. Staining may be performed on harvested cells to detect PECAM-1 (CD31) and GLUT-1. Harvested cells may be replated, such as on Transwell inserts with ECRA Medium and placed in hypoxic incubator conditions. The culture may be fed ECRA Medium every other day until confluent. Confluent cultures may be tested for transendothelial electrical resistance (TEER).

### F. Mesenchymal Cells

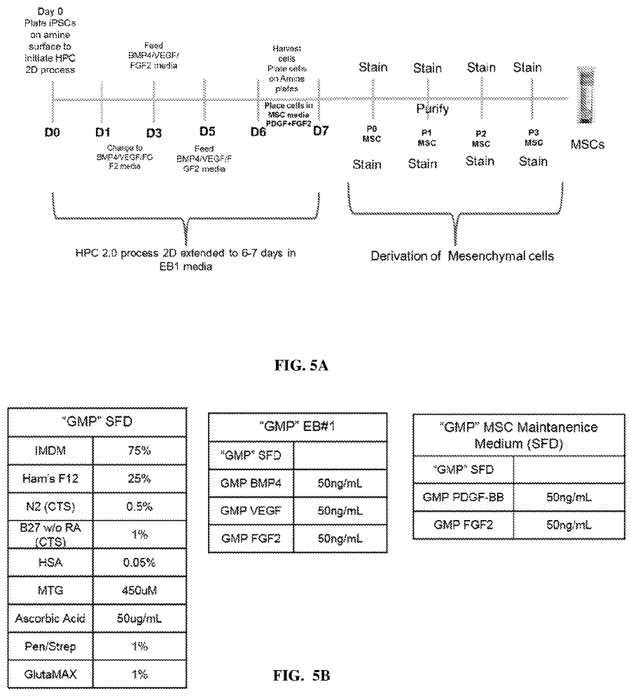

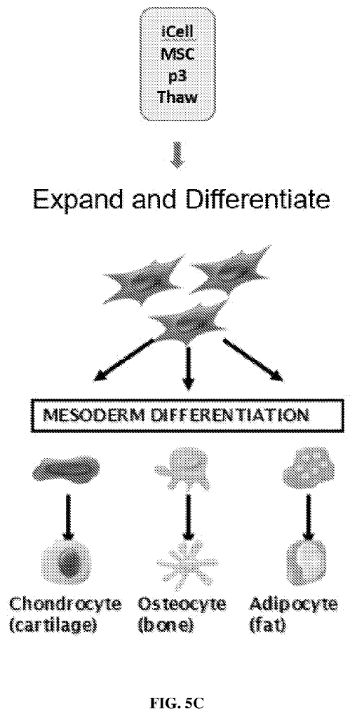

In some embodiments, the iPSCs are differentiated to MSCs. For example, FIG. 5C shows a schematic representation of the 2D HPC differentiation process to generate MSCs. iPSCs maintained on MATRIGEL™ or Vitronectin in the presence of E8, are adapted to hypoxia for at least 5-10 passages. Cells are split from sub confluent iPSCs and plated at a density of 0.25 million cells/well onto amine culture dishes in the presence Serum Free Defined (SFD) media supplemented with 5 uM blebbistatin or 10 uM H1152. 24 hrs post plating SFD media supplemented with 50 ng/ml of BMP4, VEGF and FGF2 is added to the culture. The cells are fed every 48 hrs throughout the differentiation process. The entire process is performed under hypoxic conditions. At the end of day 6 or 7 of differentiation the cells are placed in GMP-MSC media. The cells are allowed to grow to confluency and harvested at the end of each passage, then replated on an amine surface at a density of 50K/cm² in GMP-MSC Media supplemented with 5 uM blebbistatin or 10 uM H1152 to selectively allow the growth and proliferation of MSCs.

In some aspects, cryopreserved day 6 HPCs or live cultures at the end of day 6 differentiation are placed in the presence of MSC media in the presence of 10 uM H1152 on an Amine charged surface plates. The cells are given a fresh feed of MSC media 24 hrs post plating and the cultures were fed every 48 hours until they reached confluency. It took 5-6 days for cells to reach confluency. The cells are harvested using TrypLE, and stained for surface MSC markers CD73, CD44, CD105, CD49d, and the absence of Endothelial markers CD31 and CD144. The emerging cultures are passaged three times using the process described above under hypoxic conditions and amine surface. The cultures are transitioned to normoxia and normal tissue culture plates at P4.

In some aspects, the MSCs may be further differentiated to pericytes. In an exemplary method, MSCs are seeded (e.g., at a cell density of 1-20 k/cm², particularly 10 k/cm² on Tissue Culture Plastic (TCP) 6-well plates) in ScienCell Pericyte Medium (Catalog: 1201) and placed in normoxic incubator conditions. The culture may be fed ScienCell Pericyte Medium every other day until confluent. Confluent cultures may be harvested, such as by using TrypLE. Staining may be performed on harvested cells to detect neural-glial antigen 2/chondroitin sulfate proteoglycan (NG2) and PDGFR-beta (CD140b). Harvested cells may be replated (e.g., at a cell density of 1-20 k/cm², particularly 10 k/cm² on TCP 6-well plates) in ScienCell Pericyte Medium. The culture may be fed ScienCell Pericyte Medium every other day until confluent, and harvest and stained in the same manner as mentioned. The cells are then replated until there is expansion and retention of purity in cultures. Additionally, the cells may be stained positive for the presence of CD146, CD49a, CD166, CD54, CD73, CD105, CD13, CD56, CD49d, and/or CD44.

### G. Differentiation Media

Cells can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum.

Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully-defined and feeder-free media.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

### H. Cryopreservation

The cells produced by the methods disclosed herein can be cryopreserved, see for example, PCT Publication No. 2012/149484 A2, which is incorporated by reference herein, at any stage of the process, such as Stage I, Stage II, or Stage III. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C. and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 6, 7, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8% to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C./minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### III. Methods of Use

The present disclosure provides a method by which large numbers of cells of multiple lineages can be produced. These cell populations can be used for a number of important research, development, and commercial purposes. These include, but are not limited to, transplantation or implantation of the cells in vivo; screening anti-virals, cytotoxic compounds, carcinogens, mutagens, growth/regulatory factors, pharmaceutical compounds, etc., in vitro; elucidating the mechanism of liver diseases and infections; studying the mechanism by which drugs and/or growth factors operate; diagnosing and monitoring cancer in a patient; gene therapy; and the production of biologically active products, to name but a few.

### A. Pharmaceutical Compositions

Also provided herein are pharmaceutical compositions and formulations comprising the present cells and a pharmaceutically acceptable carrier.

Cell compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen.

Pharmaceutical compositions and formulations as described herein can be prepared by mixing the active ingredients (such as cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride;

benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

### B. Distribution for Commercial, Therapeutic, and Research Purposes

In some embodiments, a reagent system is provided that includes cells that exists at any time during manufacture, distribution or use. The kits may comprise any combination of the cells described in the present disclosure in combination with undifferentiated pluripotent stem cells or other differentiated cell types, often sharing the same genome. Each cell type may be packaged together, or in separate containers in the same facility, or at different locations, at the same or different times, under control of the same entity or different entities sharing a business relationship. Pharmaceutical compositions may optionally be packaged in a suitable container with written instructions for a desired purpose, such as the mechanistic toxicology.

In some embodiments, a kit that can include, for example, one or more media and components for the production of cells is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits of the present disclosure also will typically include a means for containing the kit component(s) in close confinement for commercial sale. Such containers may include injection or blow molded plastic containers into which the desired vials are retained. The kit can also include instructions for use, such as in printed or electronic format, such as digital format.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation of Endothelial Cells

iPSCs maintained on MATRIGEL™ or Vitronectin in the presence of E8, were adapted to hypoxia for at least 5-10 passages. Cells were split from sub confluent iPSCs and plated at a density of 0.25 million cells/well onto Amine culture dishes in the presence Serum Free Defined (SFD) media supplemented with 5 uM blebbistatin or 1 uM H1152. 24 hrs post plating SFD media supplemented with 50 ng/ml of BMP4, VEGF and FGF2 is added to the culture. The cells are fed every 48 hrs throughout the differentiation process. The entire process is performed under hypoxic conditions. The cells are harvested at the end of differentiation and can be cryopreserved or replated on a carboxyl surface at a density of 25k/cm² to initiate endothelial differentiation in the presence of VascuLife VEGF Endothelial Media or SFD Endothelial medium (FIG. 2).

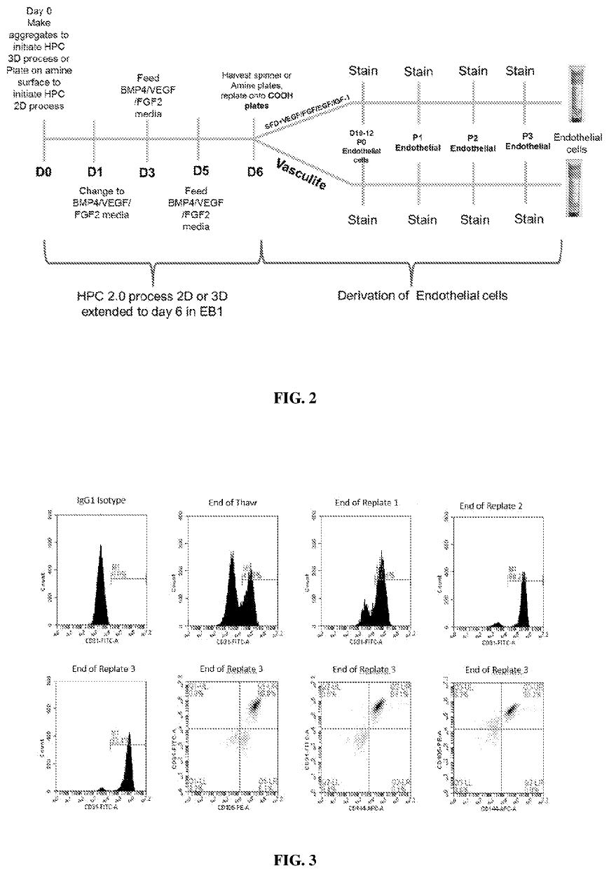

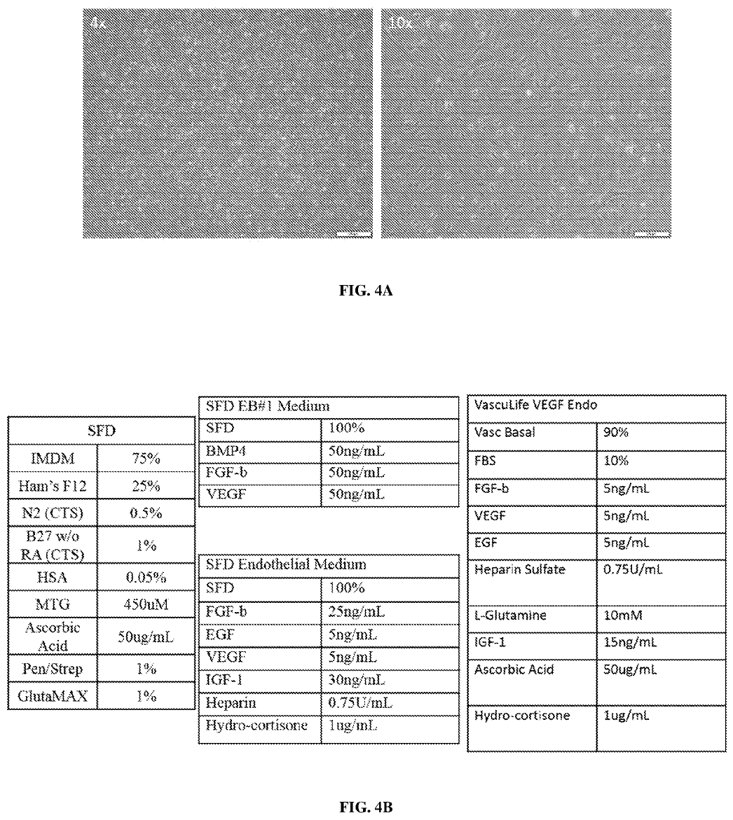

Cryopreserved day 6 HPCs or live cultures are plated at 25k/cm² on a carboxyl surface in the presence of VascuLife VEGF Endothelial Medium in the presence of 1 μM H1152 and hypoxic conditions. The cells were given a fresh feed of VascuLife 24 hrs post plating and the cultures were fed every 48 hours until they reached confluency. It took 5-6 days for cells to reach confluency. The cells were harvested using Accumax with minimal agitation or pipetting, stained for surface endothelial markers CD31, CD105 and CD144 and replated onto a carboxyl surface at 25k/cm² with VascuLife+H1152 and placed in hypoxic incubator conditions. The cells were given a full feed of VascuLife on days 2, 4, 6 post-split. On day 7, the cells were harvested, stained, and replated in the same manner three more times. The histogram depicts the increasing purity of endothelial cells at each replate stage. Pure endothelial cells were generated using successive passage purification without the use of CD31+MACS. The endothelial cells can be cryopreserved at the end of replate passage 3 (FIG. 3).

### Example 2—Generation of Mesenchymal Stem Cells

FIG. 5C shows a schematic representation of the 2D HPC differentiation process to generate MSCs. iPSCs maintained on MATRIGEL™ or Vitronectin in the presence of E8, were adapted to hypoxia for at least 5-10 passages. Cells were split from sub confluent iPSCs and plated at a density of 0.25 million cells/well onto Amine culture dishes in the presence Serum Free Defined (SFD) media supplemented with 5 uM blebbistatin or 1 uM H1152. 24 hrs post plating SFD media supplemented with 50 ng/ml of BMP4, VEGF and FGF2 was added to the culture. The cells were fed every 48 hrs throughout the differentiation process. The entire process was performed under hypoxic conditions. At the end of day 6/7 of differentiation the cells were placed in GMP-MSC media. The phenotype of the precursor population was analyzed post-harvest (FIG. 5C). The cells were allowed to grow to confluency and harvested at the end of each passage, then replated on an Amine surface at a density of 50K/cm² in GMP-MSC Media supplemented with 5 uM blebbistatin or 1 uM H1152 to selectively allow the growth and proliferation of MSCs.

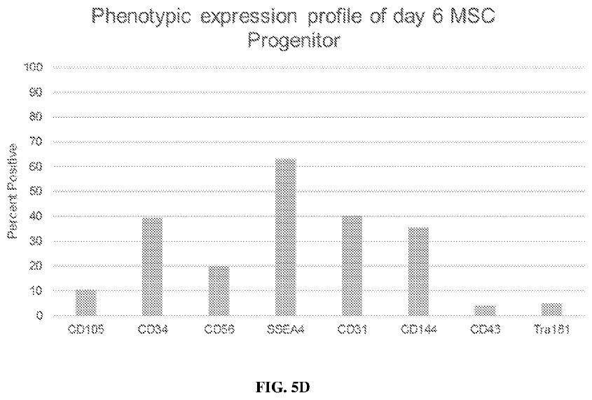

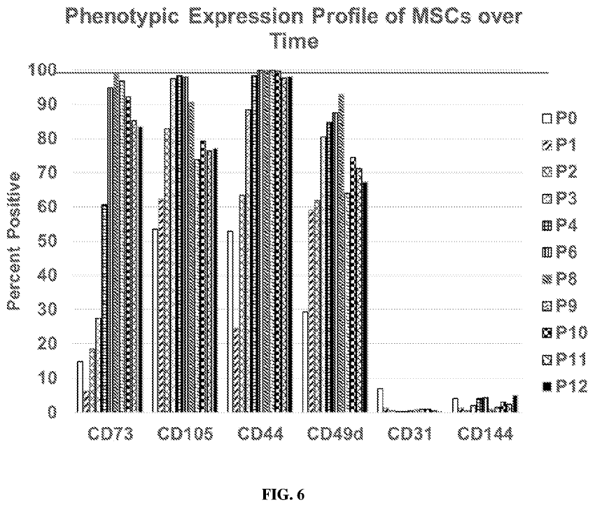

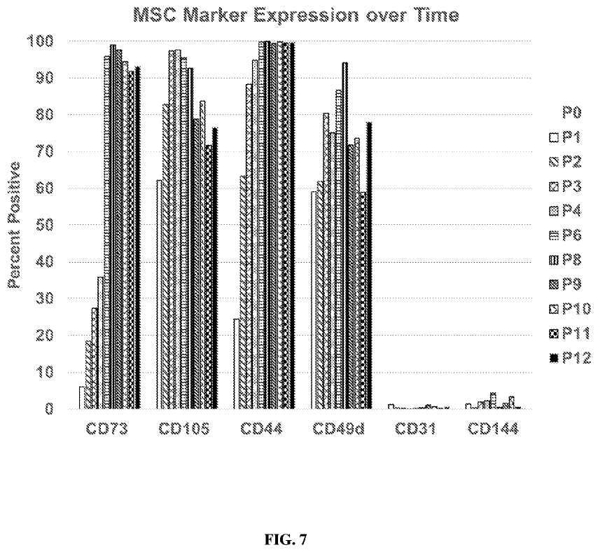

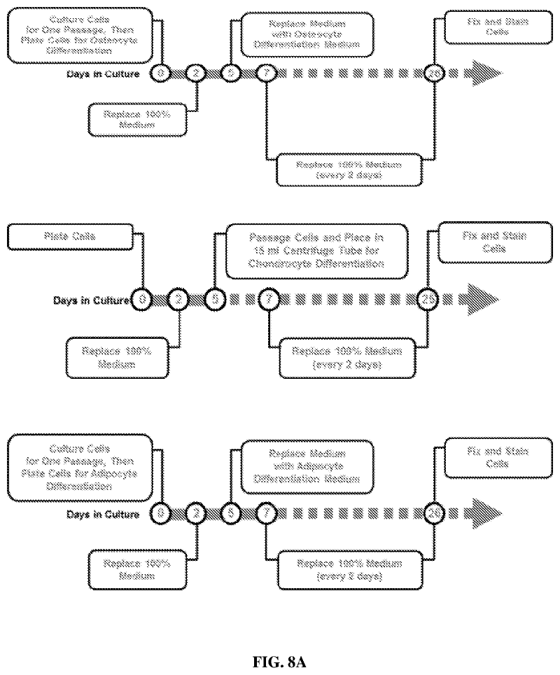

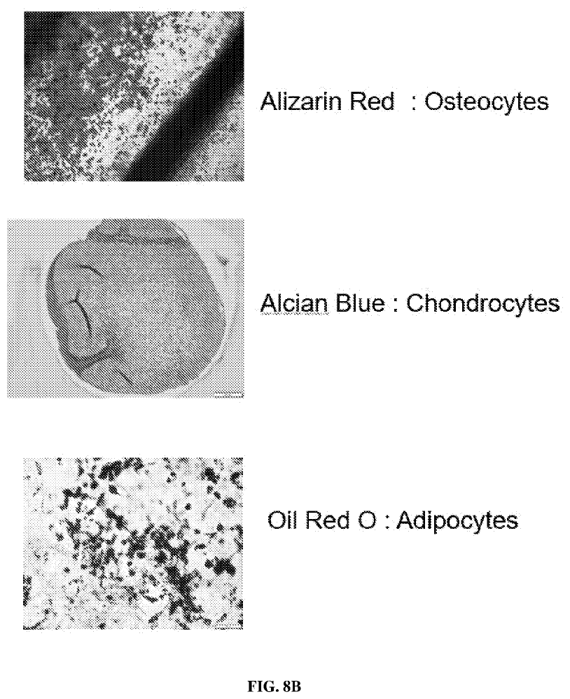

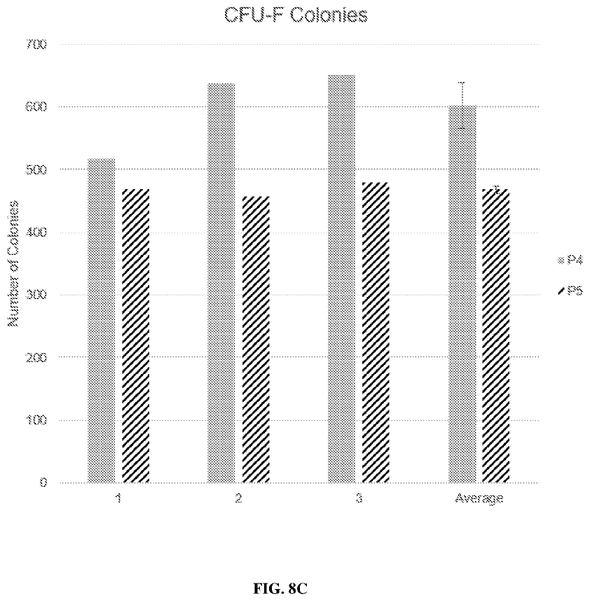

Cryopreserved Day 6 HPCs from 3D/2D HPC differentiation or live cultures at the end of day 6 differentiation emerging from 2D HPC differentiation were placed in the presence of MSC media in the presence of 1 uM H1152 on an Amine charged surface plates. The cells were given a fresh feed of MSC media 24 hrs post plating and the cultures were fed every 48 hours until they reached confluency. It took 5-6 days for cells to reach confluency. The cells were harvested using TrypLE, and stained for surface MSC markers CD73, CD44, CD105, CD49d, and the absence of Endothelial markers CD31 and CD144. The emerging cultures were passaged three times using the process described above under hypoxic conditions and Amine surface. The cultures were transitioned to normoxia and normal tissue culture plates at P4. The purity spec for MSCs was reached at P6 (FIG. 6, 7). Cryopreserved MSCs at P3 were thawed and placed in to lineage specific differentiation matrix as described in FIG. 8A to demonstrate tri-lineage potential to generate Osteocytes, Chondrocytes and Adipocytes (FIG. 8B). The clonal proliferative capacity of cryopreserved MSCs was demonstrated by plating MSCs at a density of 1000 cells/cm² in 10 cm tissue culture plates. The cells were fed with MSC media for 2 weeks with media changes every alternate day. The emerging colonies were stained with crystal violet and scored (FIG. 8C).

### Example 3—Generation of Pericytes from MSCs

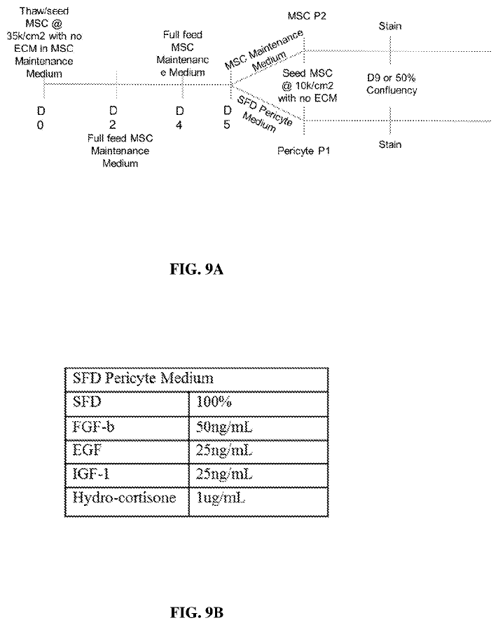

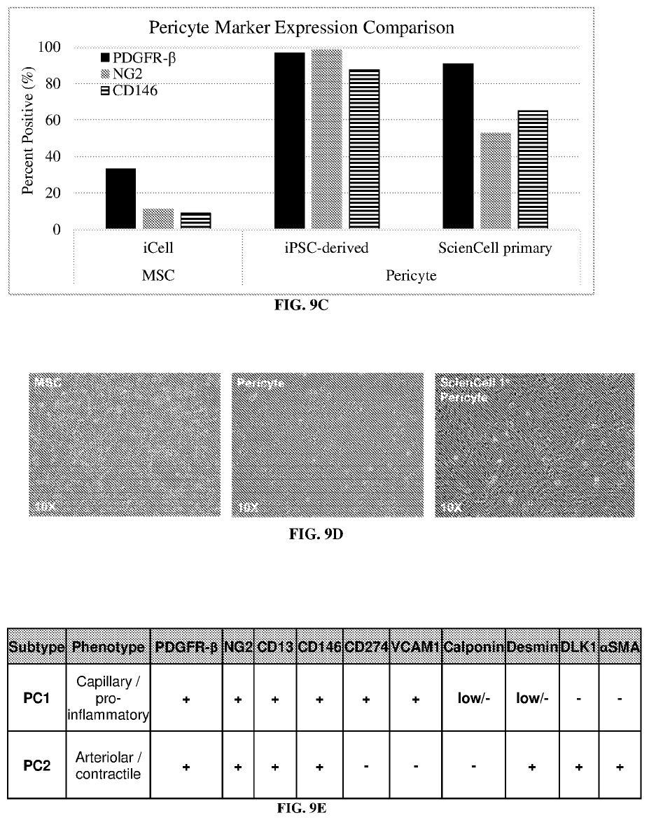

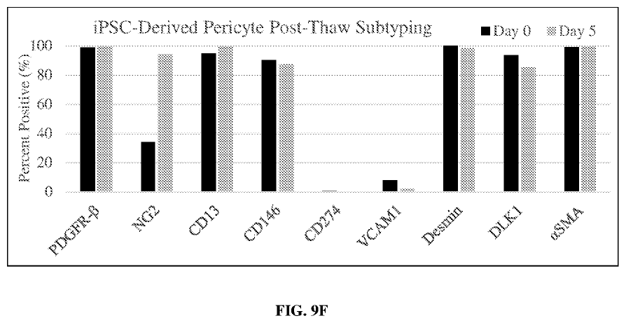

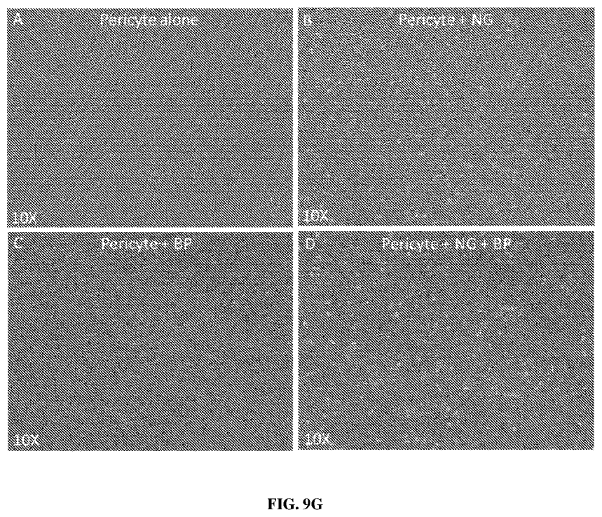

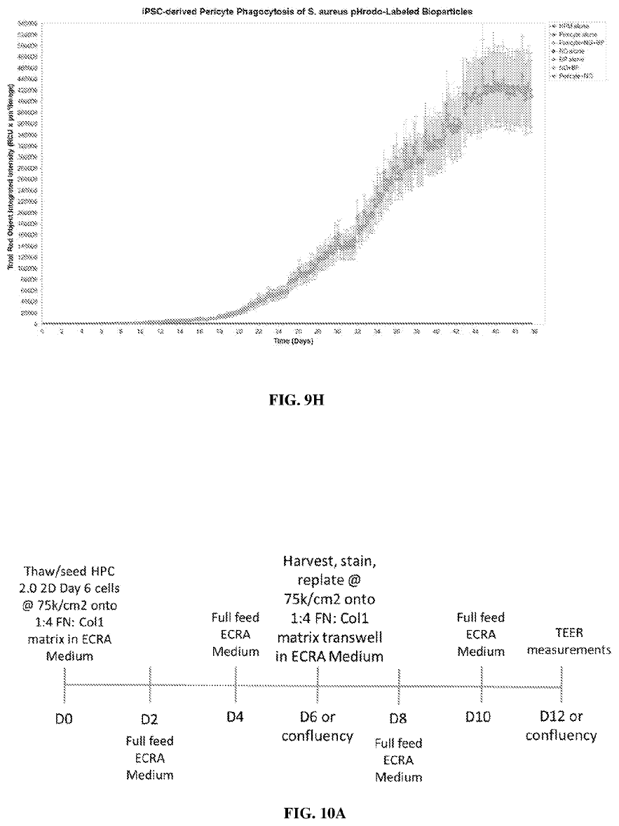

iCell MSCs and iPSC-derived pericytes were sampled at 50% confluency and analyzed by flow cytometry for known pericyte markers PDGFRβ, NG2, and CD146. Cryopreserved MSCs were thawed and plated at 35,000 cells/cm2 in 6-well plates with no extracellular matrix (ECM) in MSC Maintenance Medium (FIG. 9A). The cells were allowed to reach confluency and the cells were replated at 15,000 cells/cm2 in 6-well plates with no extracellular matrix (ECM) in SFD Pericyte Medium (SPM) (FIG. 9A; FIG. 9B).

Primary Human Brain Vascular Pericytes (HBVP) (ScienCell #1200) were thawed and plated on Poly-L-Ornithine coated 6-well plates at 5,000 cells/cm² in Pericyte Medium (ScienCell #1201). These cells were used as a positive control in the differentiation process. ScienCell HBVPs, iCell MSCs, and iPSC-derived pericytes were analyzed by flow cytometry for known pericyte markers PDGFRβ, NG2, and CD146 (FIG. 9C). There was an absence of pericyte markers of iCell MSCs at thaw. HBVP and iPSC-derived pericytes show expression of known pericyte markers PDGFRβ, NG2, and CD146, with iPSC-derived pericytes having greater purity than ScienCell HBVPs (FIG. 9C). iPSC-derived pericytes exhibit similar morphology to ScienCell HBVPs (FIG. 9D).

Based on their function, pericytes can be classified as phenotypically PC1 (pro-inflammatory) or PC2 (contractile) (Rustenhoven et al., 2017). The signature of both subtypes is described in FIG. 9E. Post-thaw, iPSC-derived pericytes were subtyped via flow cytometry for PC1 and PC2 markers CD274, VCAM1, Calponin, Desmin, DLK1, and αSMA (FIG. 9F). iPSC-derived pericytes reveal the signature of contractile pericytes, subtype PC2.

In addition to non-specific phagocytic uptake seen in chronic and acute BBB models, pericytes also specifically regulate their neuronal microenvironment by handling clearance of certain macromolecules in both physiologic and pathologic conditions (Winkler et al., 2014). iPSC-derived pericytes were plated at 15,000 cells/cm² in a 96-well plate with PDL-coating (Greiner #655946) in SPM. The cells were allowed to rest for three days post-plating before dead indicator NucGreen Dead 488 (Invitrogen #R37109) and S. aureus pHrodo Red BioParticles (Invitrogen #A10010) were added to the cells. The plate was placed on an IncuCyte live imaging system for over a month, with weekly feeds (including same concentration of live/dead and bioparticle reagents). iPSC-derived pericytes show observable phagocytic activity of S. aureus bioparticles above controls.

### Example 4—Generation of Brain Microvascular Endothelial Cells (BMECs)

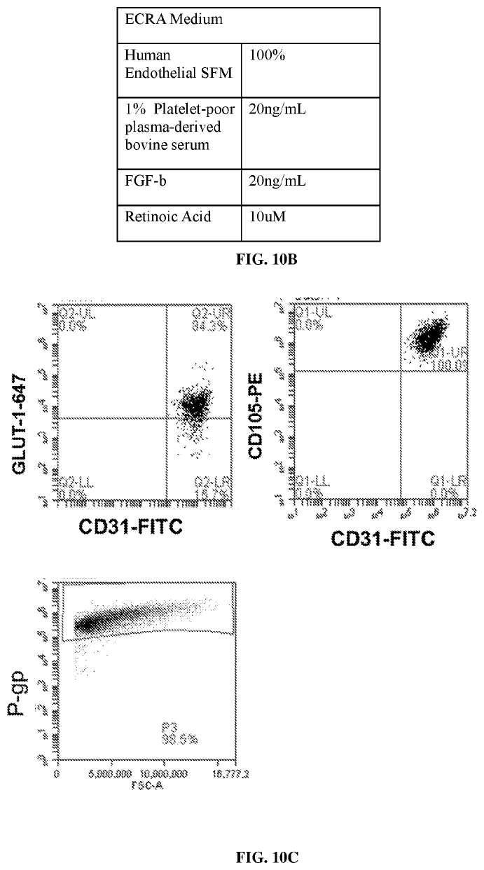

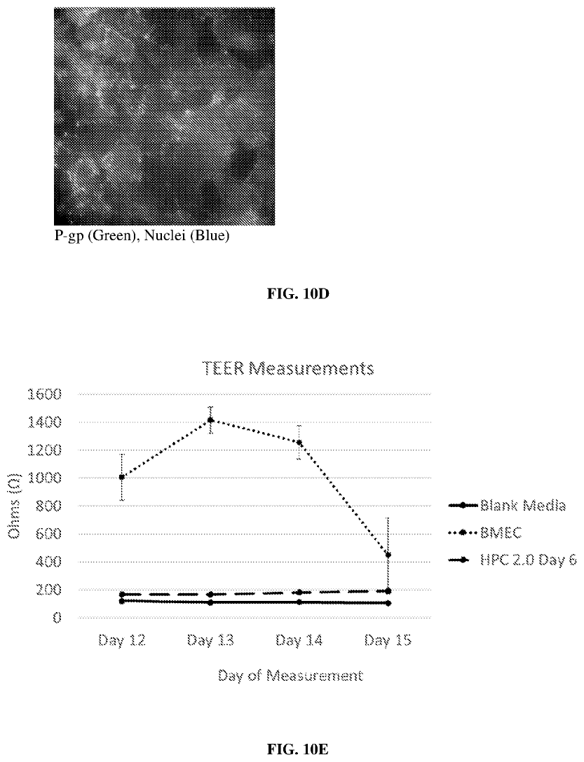

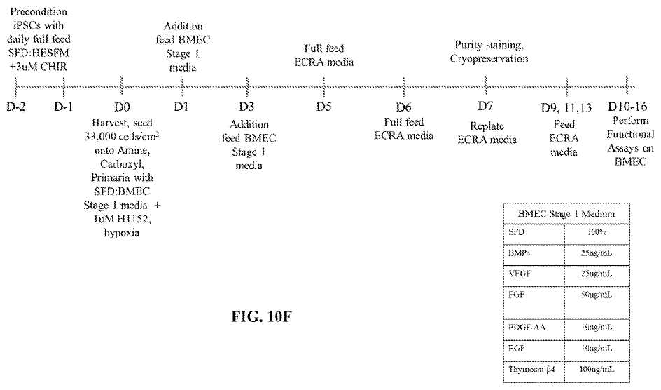

iPSCs maintained on MATRIGEL™ or Vitronectin in the presence of E8 were adapted to hypoxia for at least 5-10 passages for generating Brain Microvascular Endothelial cells. Live or cryopreserved HPCs (e.g., day 6 HPCs derived on Amine surface in the presence of SFD supplemented with BMP4, VEGF and/or FGF2, such as BMP4 and FGF2) were plated onto a ECM containing fibronectin (e.g., 50-200 μg/mL, particularly 100 μg/mL) and Collagen IV (e.g., 100-500 μg/mL, particularly 400 μ/mL) with ECRA Medium (Human Endothelial SFM (Gibco), 1% Platelet-poor plasma-derived bovine serum (Fisher), 20 ng/mL bFGF (Promega), 10uM Retinoic Acid). The cells were plated at a density of 50-100 k/cm2, particularly 75 k/cm2. The cultures were fed daily and maintained under hypoxic incubator conditions. The cultures are fed with ECRA Medium every other day until confluent. Confluent cultures are then harvested, such as by using TrypLE. Staining was performed on harvested cells to detect PECAM-1 (CD31) and GLUT-1 to confirm the identity of BMECs (FIG. 10B). Harvested cells are replated, such as on Transwell inserts with ECRA Medium and placed in hypoxic incubator conditions. (FIG. 10A). The culture may be fed ECRA Medium every other day until confluent. Confluent cultures may be tested for the presence of P-gp, CD105, Glu-1 and CD31 expression by flow cytometry (FIG. 10C) and immunocytochemistry (FIG. 10D) and trans-endothelial electrical resistance (TEER) and compared to blank media (FIG. 10E). For the immunohistochemistry cells were washed with 200 μl DPBS 3 times, then incubated with Rabbit anti-P-gp antibody (1:50 in blocking buffer (10% FBS, 0.01% TritonX in DPBS)) at 4° C. for overnight. After washing with 200 μl DPBS 3 times, P-gp was stained with secondary antibody (1:1000, Donkey anti-Rabbit IgG Alexa Fluor 488 (Invitrogen)). Nuclei was stained with Hoechst3342 (Thermo Fisher) The image was captured at 200× magnification by ImageXpress (Molecular Devices, LLC).

### Example 5—Generation of Microglia

iPSCs maintained on MATRIGEL™ or Vitronectin in the presence of E8 were adapted to hypoxia for at least 5-10 passages. 2D HPC Differentiation: Cells were split from sub confluent iPSCs and plated at a density of 0.25-0.5 million cells/well onto Amine culture dishes in the presence Serum Free Defined (SFD) media supplemented with 5 uM blebbistatin or 1 uM H1152. 24 hrs post plating SFD media supplemented with 50 ng/ml of BMP4, VEGF and FGF2 was added to the culture. The following day a full media exchange was performed.

On the fifth day of the differentiation process the cells were placed in media containing 50 ng/ml Flt-3 Ligand, SCF, TP0, IL3 and IL6 with 5U/m1 of heparin. The cells were fed every 48 hrs throughout the differentiation process. The entire process was performed under hypoxic conditions. HPCs were quantified by the presence of CD43/CD34 cells.

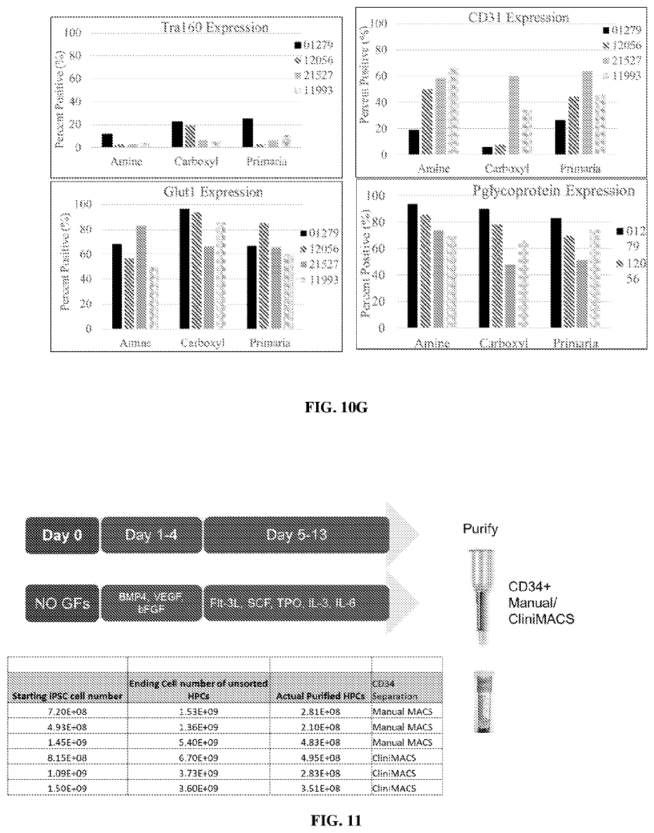

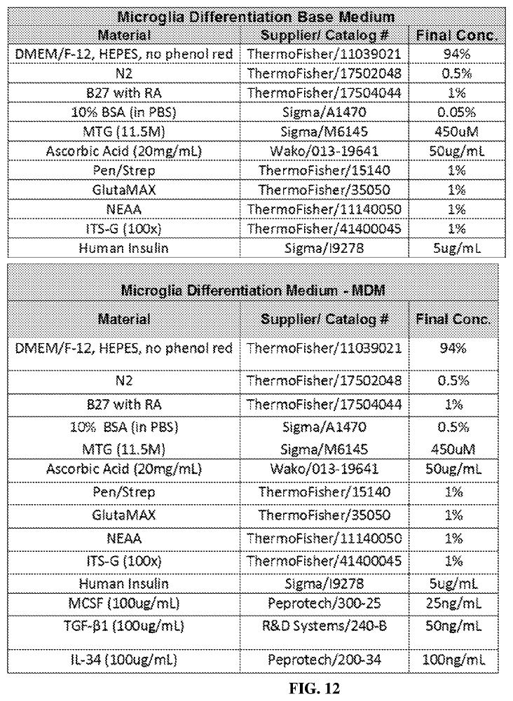

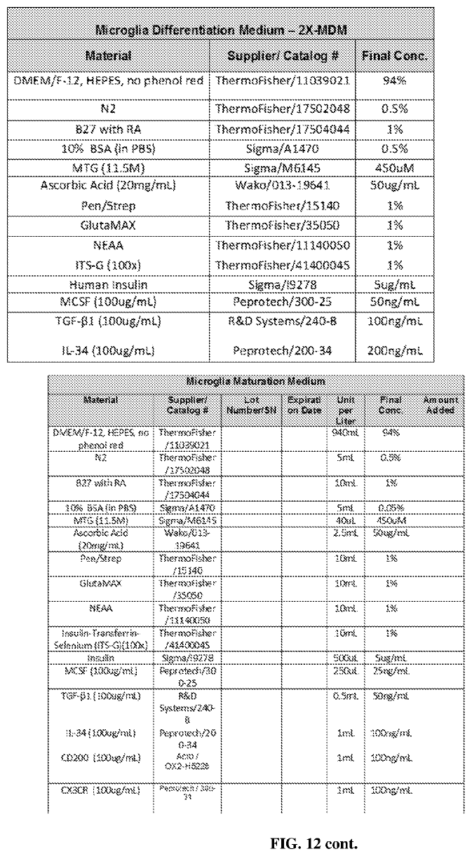

3D HPC Differentiation: Cells were split from sub confluent iPSCs and plated at a density of 0.25-0.5 million cells per ml into a spinner flask in the presence of Serum Free Defined (SFD) media supplemented with 5 uM blebbistatin or 1 uM H1152. 24 hours post plating SFD media supplemented with 50 ng/ml of BMP4, VEGF and FGF2 was exchanged. On the fifth day of the differentiation process the cells were placed in media containing 50 ng/ml Flt-3 Ligand, SCF, TP0, IL3 and IL6 with 5U/m1 of heparin. The cells were fed every 48 hours throughout the differentiation process. The entire process was performed under hypoxic conditions. HPCs were quantified by presence of CD43/CD34. The process outlines and the efficiency is illustrated in (FIG. 11) and the media compositions are listed in (FIG. 12).

HPCs were placed in microglia differentiation media MDM OR 2X-MDM (FIG. 11). The cultures were fed using every 48 hours. The purity markers for microglial cultures on day 23 of differentiation was quantified before and after cryopreservation (FIG. 13A, FIG. 13B).

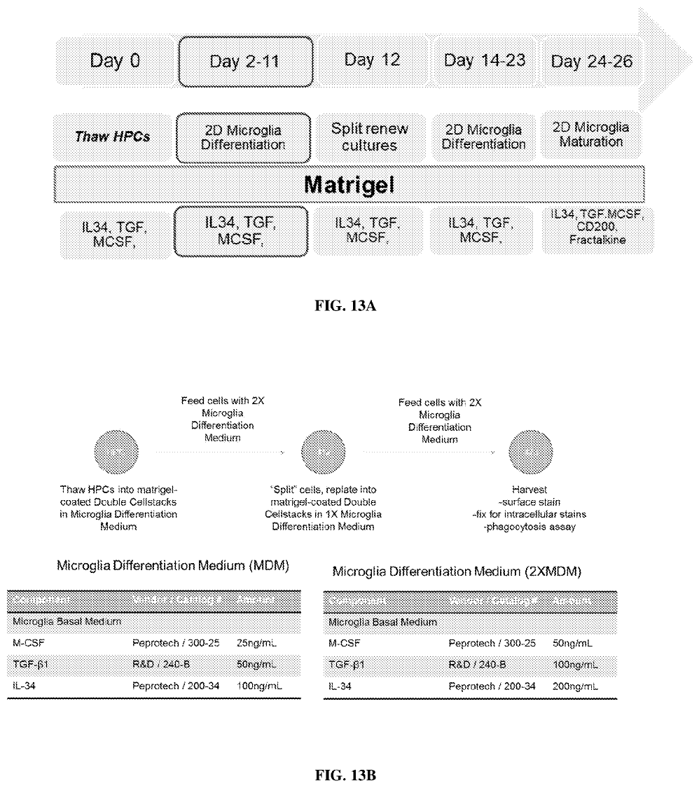

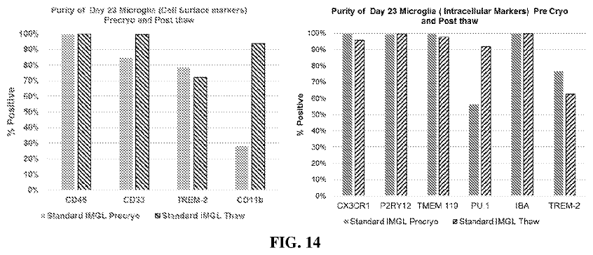

Day 23 live and cryopreserved microglia cultures were assessed for purity. Microglia cultures on day 23 differentiation were harvested and stained for the presence of microglia specific markers. The remaining cells were cryopreserved using a control rate freezer. The cryopreserved cells were thawed and stained for the presence of microglia specific markers. For both sets cell surface expression of CD45, CD33, TREM2, and CD11b (FIG. 14A) as well as intracellular expression of PU.1, IBA, P2RY12, TREM2 and TMEM119 by flow cytometry (FIG. 14B). The results revealed that cryopreserved microglia retained the purity post cryopreservation.

HPCs were placed in media to initiate microglia differentiation in the presence of MDM and the intermittent feeds were performed with 2X-MDM. The cells were cryopreserved at day 20, day 23 and day 26 of differentiation using manual freezing protocol or a control rate freezer (CRF). The cryopreserved cells were transferred to liquid nitrogen for a week. Cryopreserved microglia were thawed and placed in microglia maturation medium (MMM). The cultures were fed every 48 hours with fresh microglia maturation media. The cells were harvested on day 3, 5, 7, 10, 12 and 14-days post thaw and the recovery of viable cells with respect to the initial plating number was quantified (FIGS. 15A-15C).

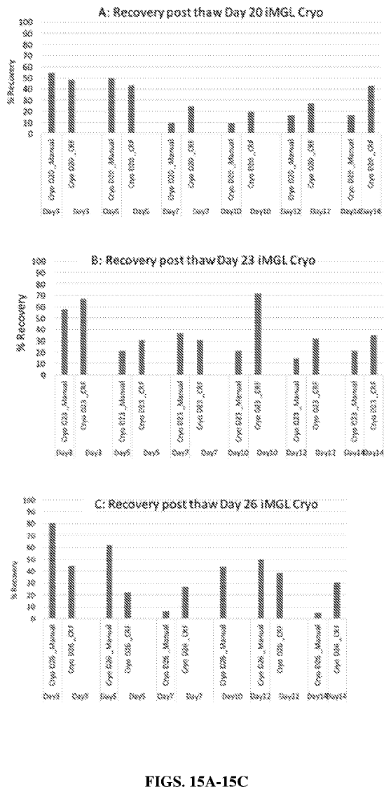

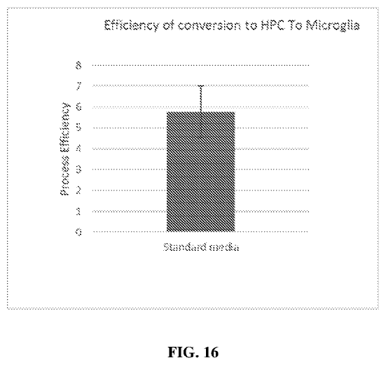

Cryopreserved HPCs were differentiated to microglia in the presence of MDM. The total viable number of input HPCs and output microglia was quantified. The process efficiency was calculated based on the purity and absolute number of TREM2 positive cells present on day 23 of microglia differentiation divided by the absolute number of input viable HPCs (FIG. 16).

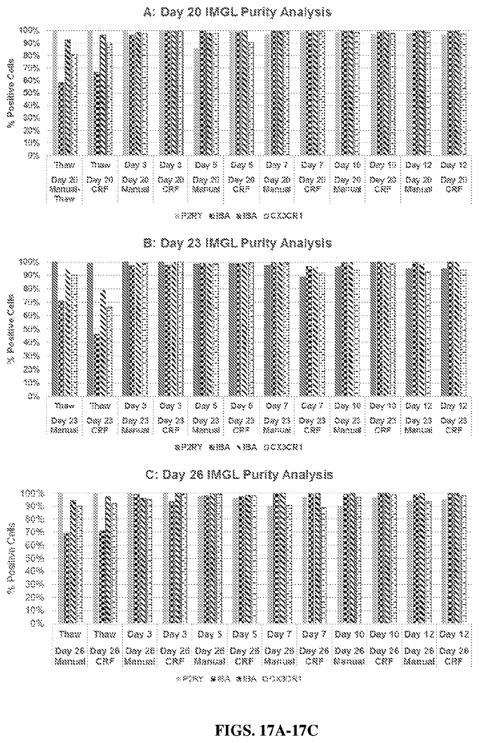

Cryopreserved microglia at day 20 (FIG. 17A), day 23 (FIG. 17B) or day 26 (FIG. 17C) of differentiation were thawed in microglia maturation medium (MMM) and fed fresh medium every 48 hours. The total viability and absolute cell number was quantified at days 3, 7 and day 10 post thaw. The data revealed a higher post thaw recovery with day 23 microglia over day 26 microglia (FIGS. 17A-17C).

Next, functional assessment was performed on cryopreserved microglia on day 20, day 23 and day 26 of the differentiation process. The cells were thawed and plated at 15,000 viable cells/well in a 96 well plate in the presence of 200 μl Microglia Maturation medium per well. The cells were treated with diluted 1 μg/well of opsonized or non-opsonized pHrodo Red BioParticles (Thermo Fisher #A10010, 2 mg per vial; stored at −20° C.). The plate was placed on the IncuCyte and images of the phagocytosis were taken at various time points up to 5 days post thaw. Cells cryopreserved via control rate freezer method exhibit more robust phagocytosis (due to higher cell viability (FIG. 19)).

The functional assessment was extended to later time points post thaw. The phagocytic potential was assessed at day 5, day 7 and day 14 post thaw for cryopreserved microglia at day 20, 23 and 26 day of differentiation using the manual or control rate freezer assessed via live imaging on the IncuCyte system. Cryopreserved microglia were thawed and plated in MMM for three days. The viable cell counts at the end of three days were determined as described in FIG. 18B. 15,000 viable cells were plated in a 96 well plate in the presence of 200 μl Microglia Maturation medium (MMM) per well with diluted 1 μg/well of opsonized or non-opsonized pHrodo Red BioParticles and the plate was placed on the IncuCyte and images of the phagocytosis were taken at various time points up to 5, 7 and 14 days post thaw. Manual cryopreservation method revealed decreased/right-shifted rate of phagocytosis across all conditions (due to lower cell viability).

Phagocytic index is a measure of phagocytic activity determined by counting the number of bacteria ingested per phagocyte during a limited period of incubation of a suspension of bacteria and phagocytes. The ability of cryopreserved microglia to engulf labeled bacterial particles was quantified by the ratio of the number of phagocytosis red object count/total live cells. This ratio was determined as the phagocytic index (FIG. 21).

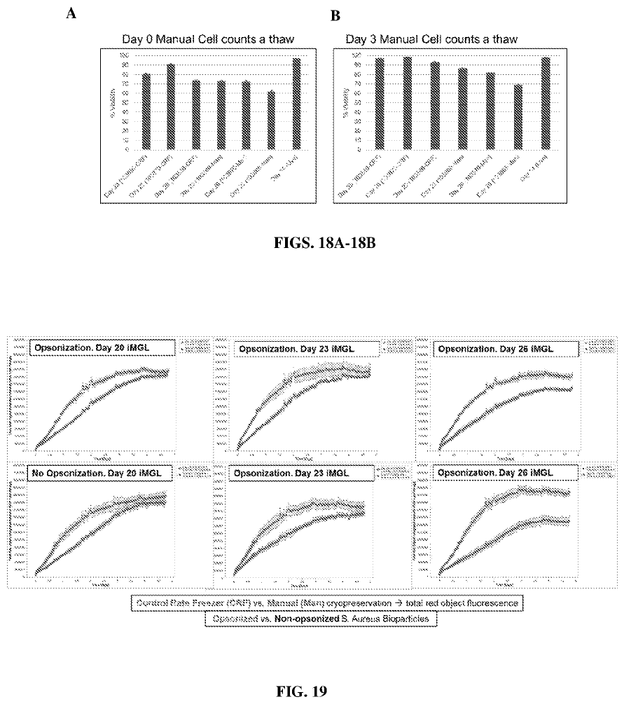

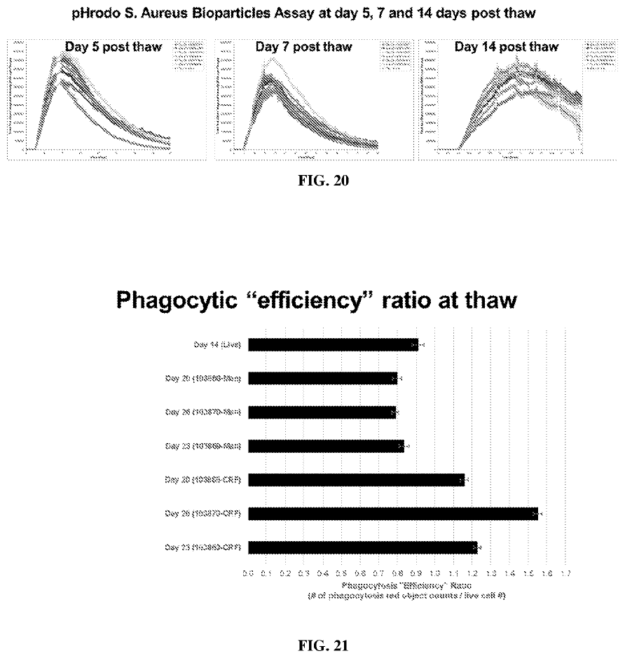

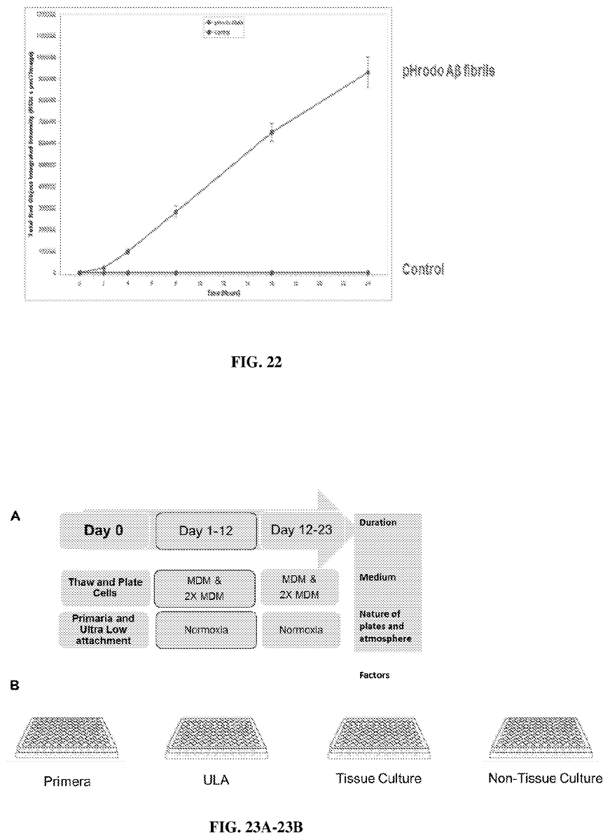

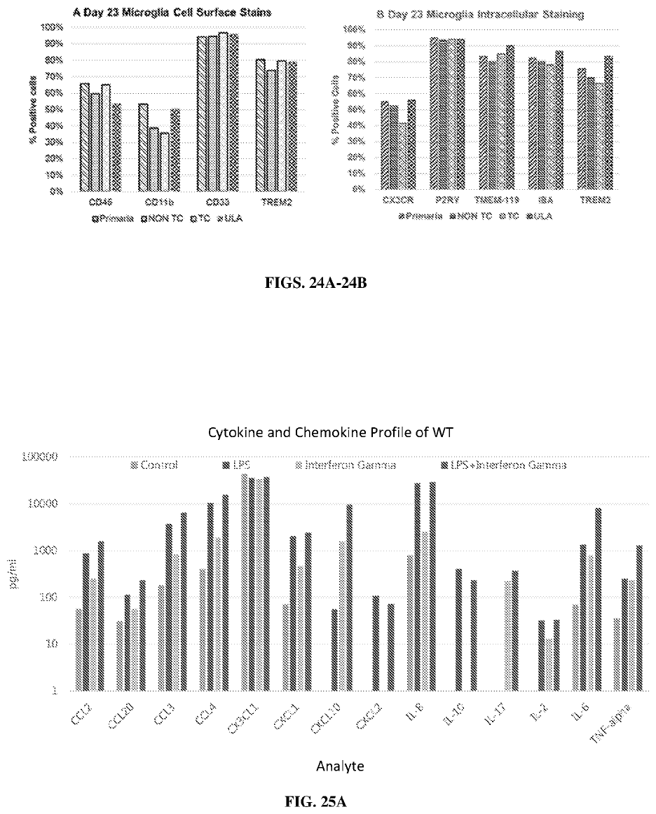

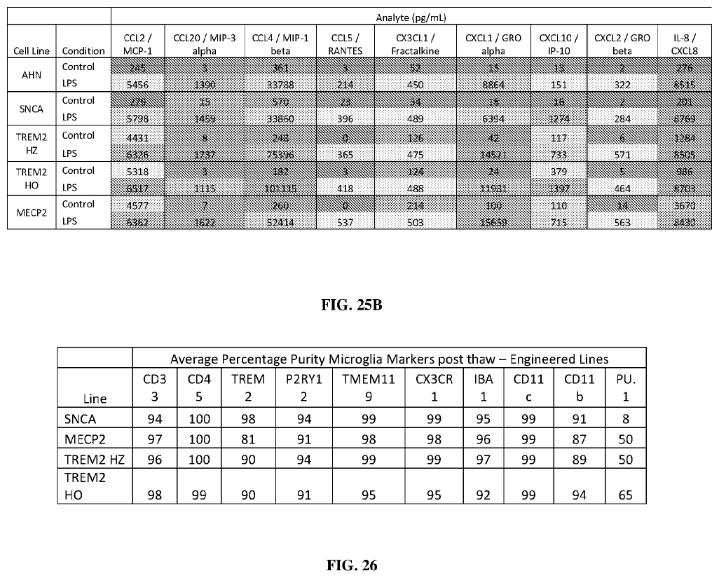

Cryopreserved microglia were thawed and plated at 15,000 viable cells/well in a 96 well plate in the presence of 200 μl Microglia Maturation medium per well. The cells were treated with diluted 1 μg/well of opsonized or non-opsonized pHrodo Red BioParticles (Thermo Fisher #0 A10010, 2 mg per vial; stored at −20° C.). The plate was placed on the IncuCyte and images of the phagocytosis were taken at various time points up to 5 days post thaw. Cells cryopreserved via control rate freezer method exhibit more robust phagocytosis (due to higher cell viability (FIG. 22).

Next, the differentiation of HPCs to microglia was further developed in the absence of ECM and in a 96-well format amenable to screening application. The differentiation as performed on Ultra-low attachment (ULA), Tissue Culture (TC) and Non-tissue culture (Non-TC) vessels (FIG. 23A). Cryopreserved HPCs were plated at a density of 20,000-35,000 viable cells/cm2 on a 96 well Primaria plate or Ultra-low attachment, tissue culture (TC) or non-tissue culture plates (Non-TC) in the presence of 200 ul microglia differentiation medium per well (FIGS. 23B). The cells were fed every 48 hrs with 50 μl media per well of MDM for the next 23 days of differentiation. The cells were harvested with cold PBS on day 23 and the total viable cell number was quantified using an automated cell counter. The cells were stained for surface expression of CD11b, CD45, CD33, TREM2 and intracellular expression of TREM2, IBA, P2RY12 and TMEM119 (FIGS. 24A-24B).

TABLE 1
Process Efficiency of generating microglia on charged surfaces.

| Plate Type | Day 0 Cell Number | Day 23 Cell Number | Expansion |
|---|---|---|---|
| Primera | 0.684 × 106 | 4.0 × 106 | 5.8x |
| ULA | 0.684 × 106 | 2.06 × 106 | 3x |
| TC | 0.684 × 106 | 3.22 × 106 | 4.7x |
| Non-TC | 0.684 × 106 | 3.52 × 106 | 5.1x |

Cytokines and Chemokines released by cryopreserved microglia. Day 23 cryopreserved microglia were thawed into MDM medium and plated on to Primaria 96 well plate at 50,000 cells/well. Cells were plated for three days prior to starting stimulations with 100 ng/ml LPS and 50 ng/ml interferon gamma Stimulations were performed in triplicate for 24 hours. Supernatant was spun down to remove cells and debris and immediately placed at −20° C. The supernatants were analyzed on a multiplex Luminex assay.

### Example 6—Engineering iPSCs to Generate Variants Mimicking Neurodegenerative Diseases

TREM2 function was disrupted by introducing indels in exon 2, leading to frameshift and premature translation termination. TAL-nucleases (pair TREM2 below) were designed to bind DNA sequences centered around amino acid 58 within exon 2. The cell line used for engineering was the FCDI iPSC line 01279.107. TAL nuclease mRNA and a co-selection plasmid expressing blasticidin resistance under the control of the SV40 promoter were electroporated into cells using a BioRad Gene Pulser Xcell system with settings of 125V/950uF. The cells were plated and a short blasticidin selection was applied on days 1 and 2 post electroporation. Surviving cells were grown and then single-cell sorted into 96 well plate on day 7 post electroporation. After about two weeks, 81 clones were picked and genotyped by PCR and sequencing.

Of 81 clones sequenced, 7 showed sequence modification. Three clones contained one allele with an insertion of one base pair, three clones contained one allele with a deletion of one base pair, and one clone was a compound heterozygote with one allele containing a one base pair insertion and one allele containing a four base pair deletion. The seventh clone contained a deletion of 24 base pairs that was not expected to introduce a frameshift. Clones were expanded, cryopreserved, and underwent sequence confirmation and karyotype analysis. After differentiation into microglia two main clones were chosen as example Heterozygous or Homozygous disruptions. The Heterozygous clone 01279.1185 contained an allele with the 1 bp insertion, leading to a frameshift at position 60 of TREM2 and termination after 45 following amino acids. The Homozygous clone 01279.1187 contained an allele with a 1 bp frameshifting insertion at position 59 and termination 16 amino acids later, and the second allele having a 4 bp deletion at position 59 leading to a frameshift and termination 46 amino acids later.

TABLE 2
Target sequences.

| TAL | Target Sequence |
|---|---|
| TREM2-F | GGTGCCGCCAGCTGGGAG |
| TREM2-R | GCGTGCTGACCACACGCT |

TABLE 3

| Name of Cell | | | |
|---|---|---|---|
| Line | | DNA variant | |
| (Descriptive) | iPSC Number | Description | Predicted Protein variant description |
| TREM2 | 01279.1185 | NM_018965.3:c.[280dupC]; | NP_061838.1:p.[(Cys60MetfsTer45)]; |
| Heterozygous | | [280_281=] | [(=)] |
| KO | | | |
| TREM2 | 01279.1187 | NM_018965.3:c.[278_279insG]; | NP_061838.1:p.[(Pro59AlafsTer16)]; |
| Homozygous | | [278-281delCCCA] | [(Pro59AlafsTer46)] |
| KO | | | |

### Example 7—Generation of Additional Isogenically Engineered Lines Mimicking Neurodegeneration

A Parkinson's Disease model was produced by genetically engineering episomally reprogrammed iPSC 01279 by nuclease-mediated homologous recombination and a donor oligo SJD 14-133. The resulting iPSCs contained SNP rs104893877 where amino acid 53 was changed from alanine to threonine resulting in the A53T variant in the alpha-synuclein gene (SNCA) as well as two silent mutations resulting in the SNCA A53T iPSC line.

An isogenically engineered model for studying Rett Syndrome was generated by using nuclease-mediated homologous recombination and a donor plasmid p1553. Donor plasmid p1553 inserted a series of stop codons prior to the Methyl CpG Binding domain followed by a PGKp-PuromycinR-SV40pA selection cassette flanked by LoxP sites. The MECP2 HM line derived from parental line 01279 provided a disease model for Rett Syndrome.

Generation of HPCs and microglia from isogenic engineered iPSCs: Homozygous and Heterozygous TREM2 KO iPSCs derived from 01279 iPSC along with SNCA A53T and MECP2 HM engineered lines derived from 01279 were maintained in the presence of E8 and MATRIGEL™ acclimatized to hypoxic conditions by passaging them for 10 passages. The cells were karyotyped, and iPSC banks were made to initiate HPC differentiation via the 3D HPC differentiation protocol. Cells were split from sub confluent iPSCs and plated at a density of 0.25-0.5 million cells per ml into a spinner flask in the presence of Serum Free Defined (SFD) media supplemented with 5 μM blebbistatin or 1 μM H1152. 24 hrs post plating SFD media supplemented with 50 ng/ml of BMP4, VEGF and FGF2 is exchanged. On the fifth day of the differentiation process the cells are placed in media containing 50 ng/ml Flt-3 Ligand, SCF, TP0, IL3 and IL6 with 5-10 U/ml of heparin. The cells are fed every 48 hrs throughout the 13 day differentiation process. The entire process is performed under hypoxic conditions. HPCs quantified by presence of CD43/CD34. HPCs are cryopreserved post MACs sorting using CD34 beads. Microglia were generated by thawing cryopreserved HPCs and placing the cells in a 23-day differentiation process as described in Example 5.

Cryopreserved microglia from day 23 wild type and TREM engineered clones were thawed and the presence of TREM-2 expression along with CD45 was quantified by flow cytometry (FIG. 26).

Cryopreserved day 23 microglia derived from isogenically engineered lines were thawed and stained for the presence of microglia specific markers. The cells were stained to quantify cell surface expression of CD45, CD33, TREM2, and CD11b as well as intracellular expression of PU.1, IBA, P2RY12, TREM2 and TMEM119 proteins by flow cytometry. FIG. 26 summarizes the purity obtained across all four isogenically engineered iPSCs. The results demonstrate the generation of highly pure microglia from isogenically engineered iPSCs with no alteration in the differentiation protocol.

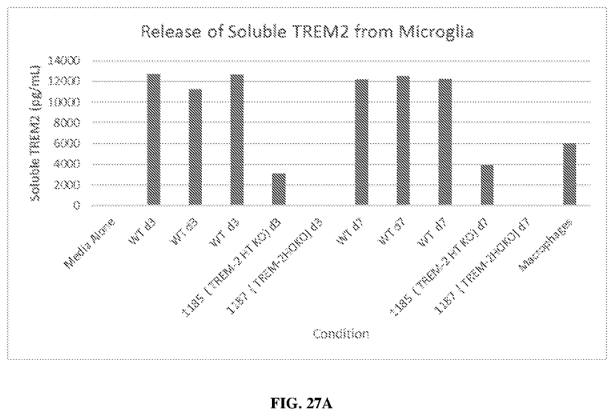

Levels of soluble TREM2 (sTREM2) protein secreted by microglia post thaw were quantified from conditioned media collected from WT and TREM2 Heterozygous and Homozygous KO mutants using a Simple Step ELISA (AbCam) (FIG. 27A). WT and TREM2 KO microglia were thawed and plated at the same density in maturation media in a 96 well Primaria plate. The spent media was collected at Day 3 post thaw and day 7 post thaw.

The cultures were half fed with fresh maturation media on day 3 and Day 5 post thaw. The data revealed a difference in the levels of soluble TREM2 between WT, Heterozygous TREM2 KO and Homozygous KO microglia. This assay can be used as a functional assay to distinguish WT and TREM2 engineered iPSCs.

Levels of soluble TREM2 (sTREM2) protein secreted by microglia post thaw were quantified from conditioned media collected from WT and TREM2 Heterozygous and Homozygous KO mutants, MECP2HM and SNCA-A53T using a Simple Step ELISA (AbCam) (FIG. 27A). WT and TREM2 KO microglia were thawed and plated at the same density in maturation media in a 96 well Primaria plate. The spent media was collected at Day 3 post thaw and day 7 post thaw. The cultures were half fed with fresh maturation media on day 3 and Day 5 post thaw. The data revealed a difference in the levels of soluble TREM2 between WT, Heterozygous TREM2 KO and Homozygous KO microglia. This assay can be used as a functional assay to distinguish WT and TREM2 engineered iPSCs. The release soluble TREM2 was impaired in A53T-SNCA microglia while MECP2HM microglia did reveal any alterations in the levels of sTREM released in the media (FIG. 27B).

Cytokines and Chemokines released by isogenically engineered cryopreserved microglia. Day 23 cryopreserved microglia derived from WT, 1185 HT TREM2 KO, 1187 HO TREM2 KO A53T-SNCA and, MeCP2HM microglia were thawed into MDM medium and plated on to Primaria 96 well plate at 50,000 cells/well. Cells were plated for three days prior to starting stimulations with 100 ng/ml LPS to check M1 mediated response.

Stimulations were performed in triplicate for 24 hours. Supernatant was spun down to remove cells and debris and immediately placed at −20° C. The supernatants were analyzed on a multiplex Luminex assay. The results of this multiplex Luminex assay is captured as a heat map in FIG. 27C. Engineered lines secreted a higher level of IL-6 compared to ANH control. TREM2 HZ and TREM2 HO and MeCP2HM microglia released less TNF alpha, but increased levels of IL6 compared to ANH. A53T-SNCA microglia released similar levels of IL-6 and TNF alpha compared to AHN control microglia.

All engineered lines released the M2 cytokine IL-10 when treated with an M1 stimulus (LPS). MECP2HM microglia released less IL-10 compared to the AHN control microglia (FIG. 27E). AHN and engineered microglia were capable to release CCL2/MCP-1, CCL20/MIP-3 alpha, CCL4/MIP-1 beta, CCL5/RANTES, CX3CL1/Fractalkine, CXCL1/GRO alpha, CXCL10/IP-10, CXCL2/GRO beta, IL-8/CXCL8 in response to LPS stimulation. There were some inherent differences in the level of cytokines releases. TREM2HO revealed the highest levels of CCL4 which is key analyte released during the onset of Alzheimer' s Disease (AD). MECP2HM, TREM2HZ and TREM2HO microglia released higher levels of CXCL1/GRO which implies an attempt to recruit assisting cell types granulocytes to aid in microbial killing and trigger an inflammatory response during phagocytosis, MECPHM microglia revealed a spontaneous secretion of IL-8/CXC18. This analyte is elevated in brain injury and induces the expression of pro-inflammatory proteases and MMP-2 and MMP-9. MECPHM microglia secreted higher levels of IL-6. These results suggest that MECPHM are primed for a pro-inflammatory response. Engineered and AHN microglia released similar levels of PDL-1, CD40, FLT-3 and PDGFAA in the media in response to LPS.

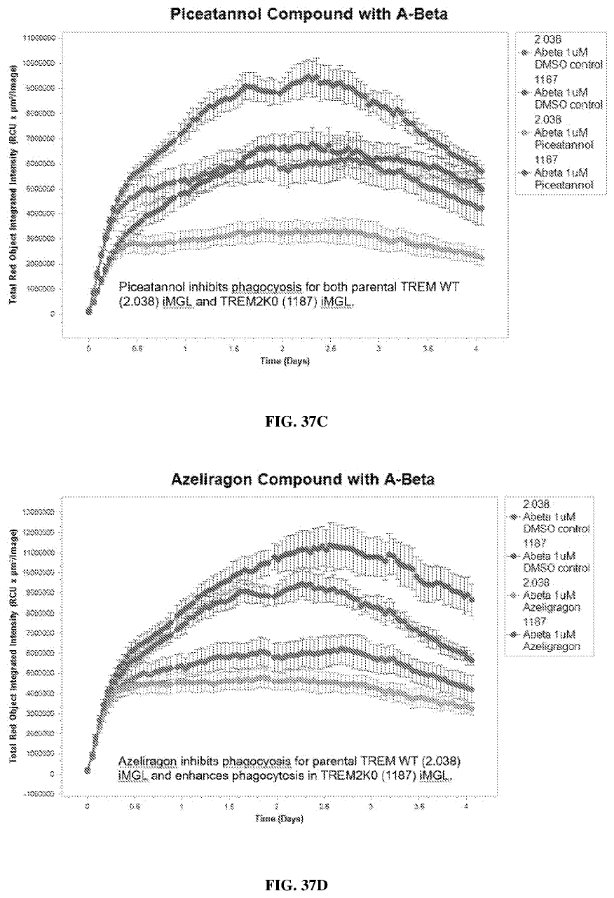

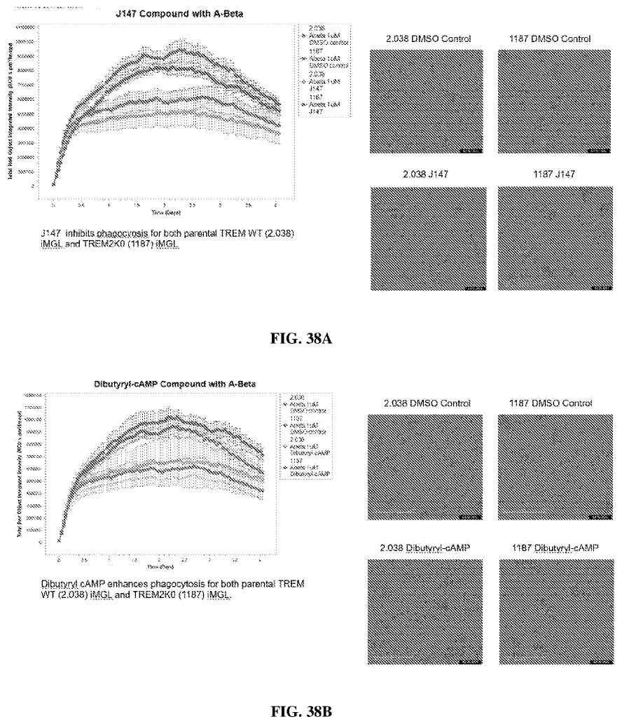

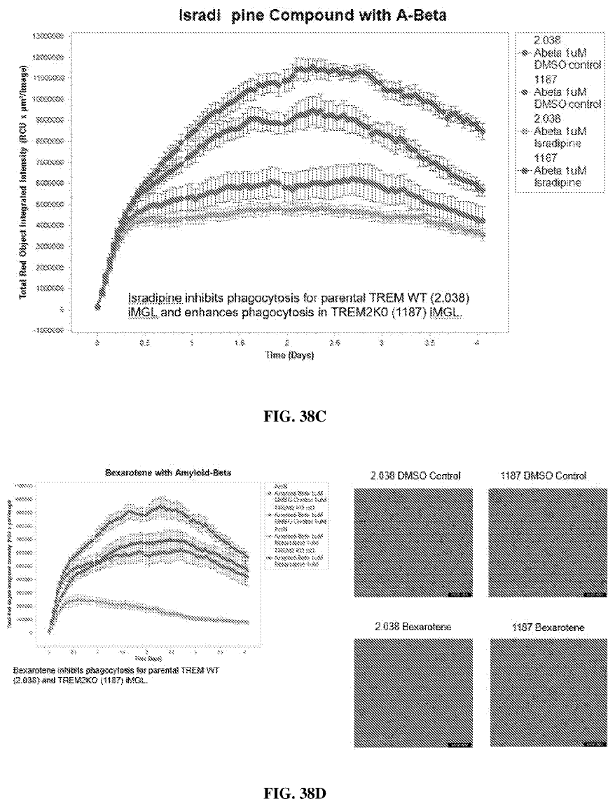

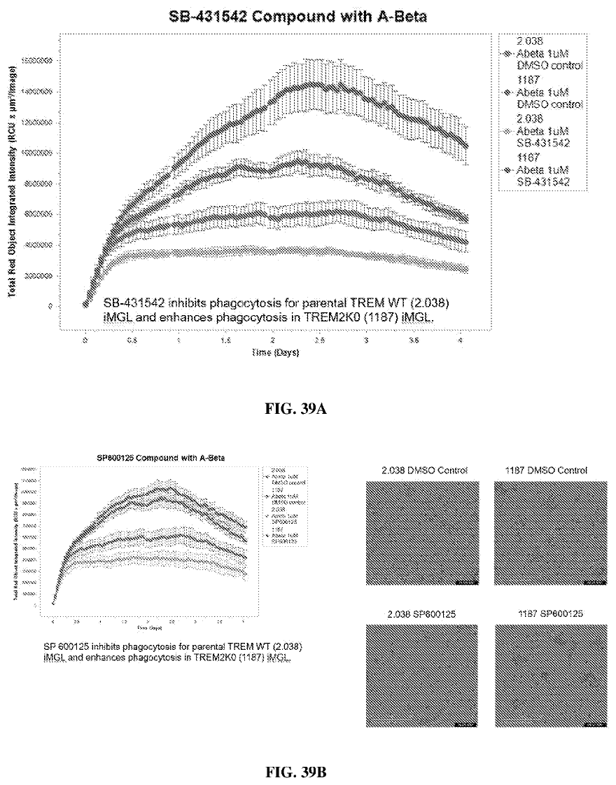

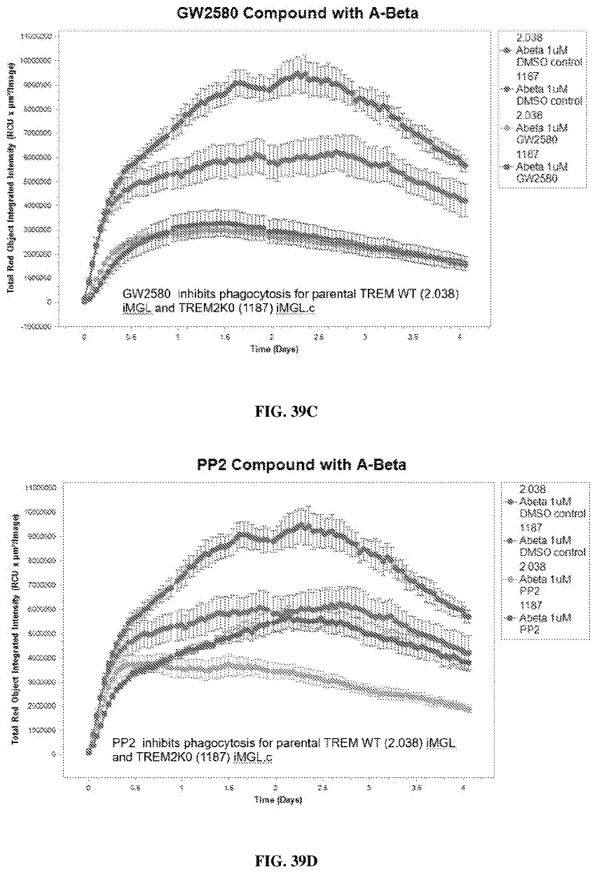

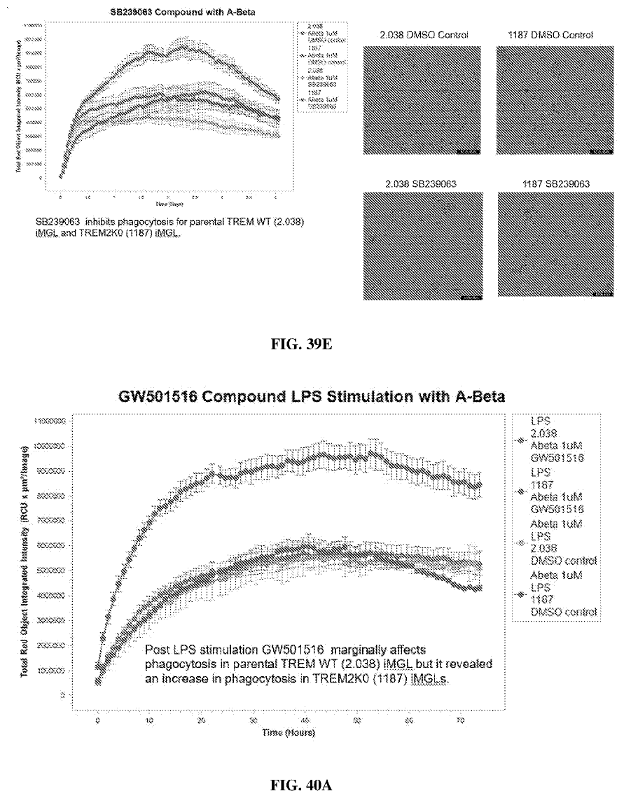

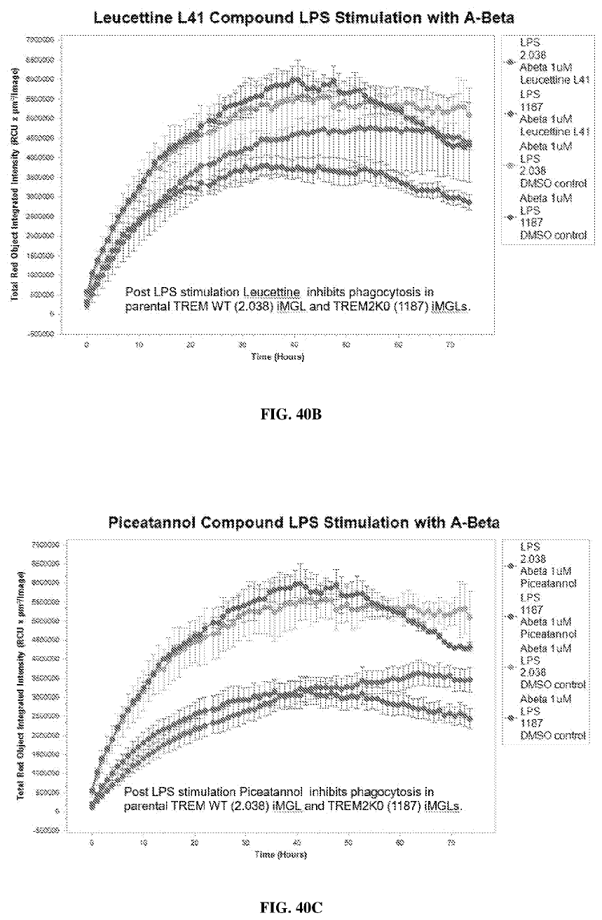

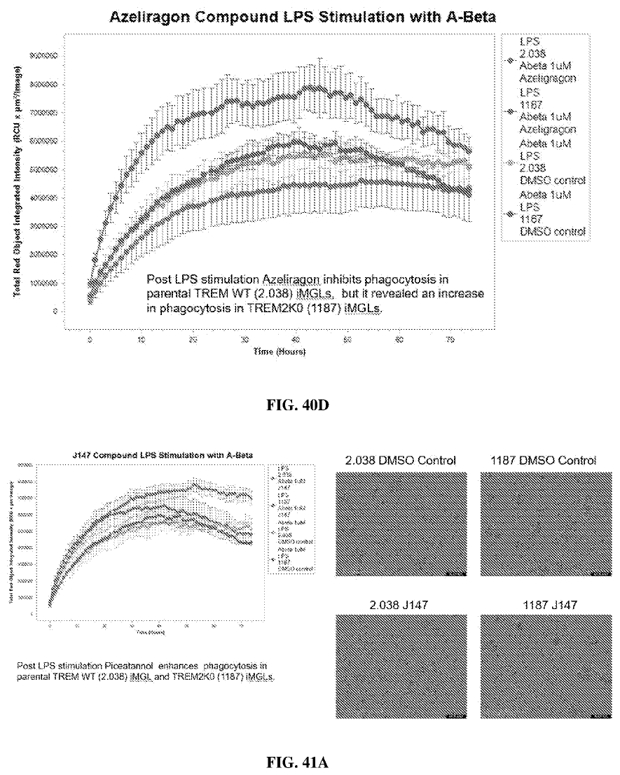

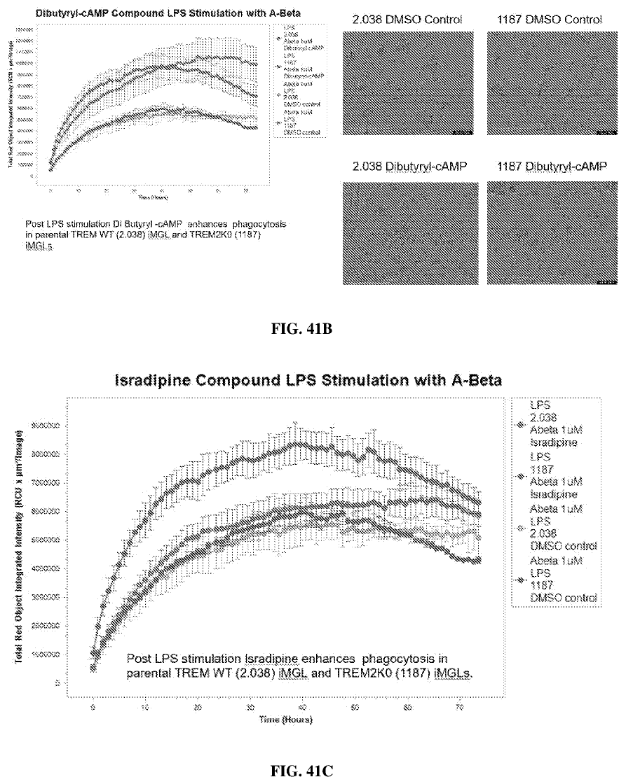

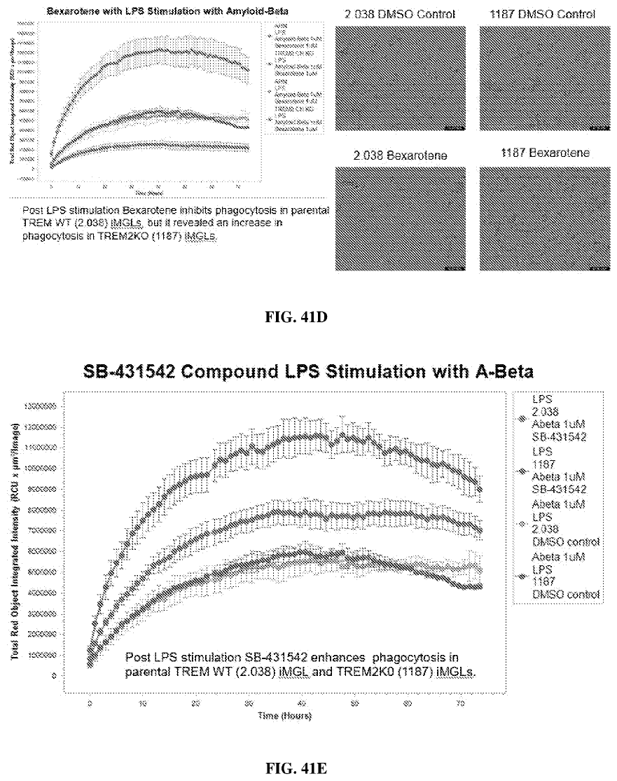

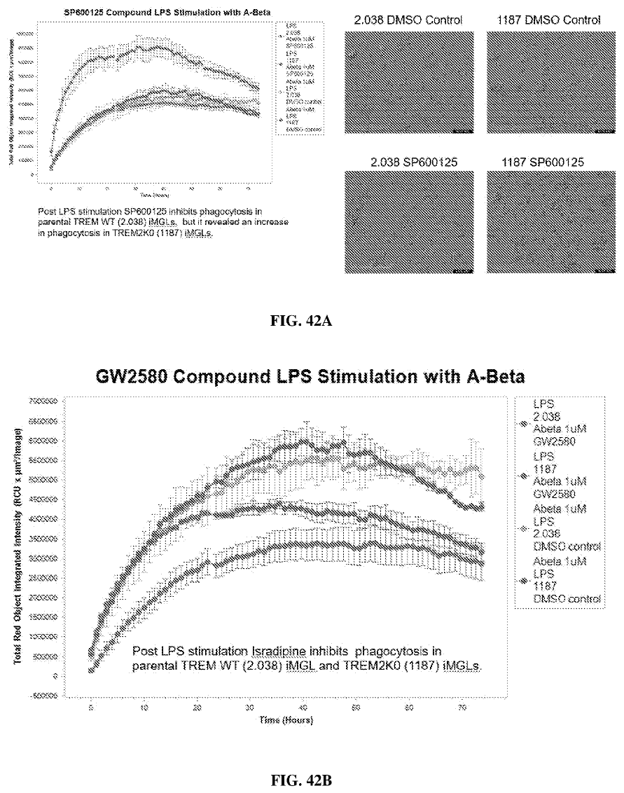

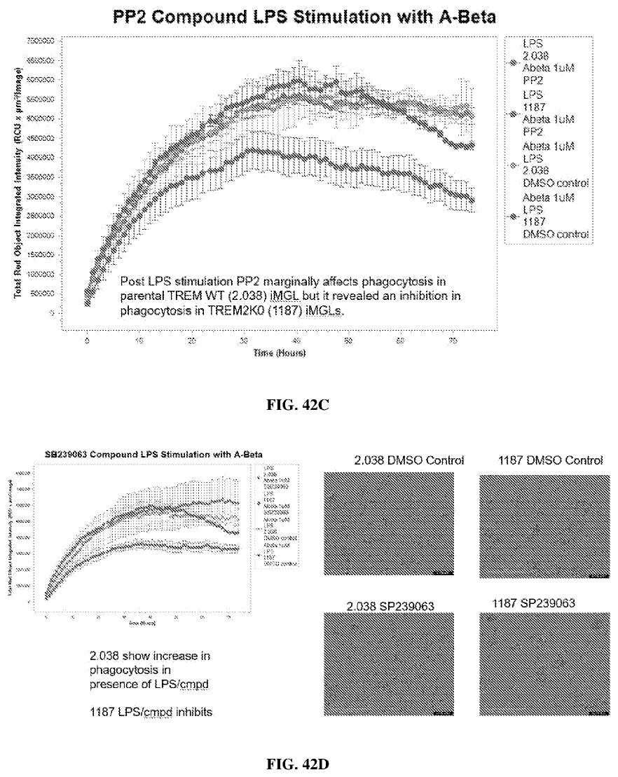

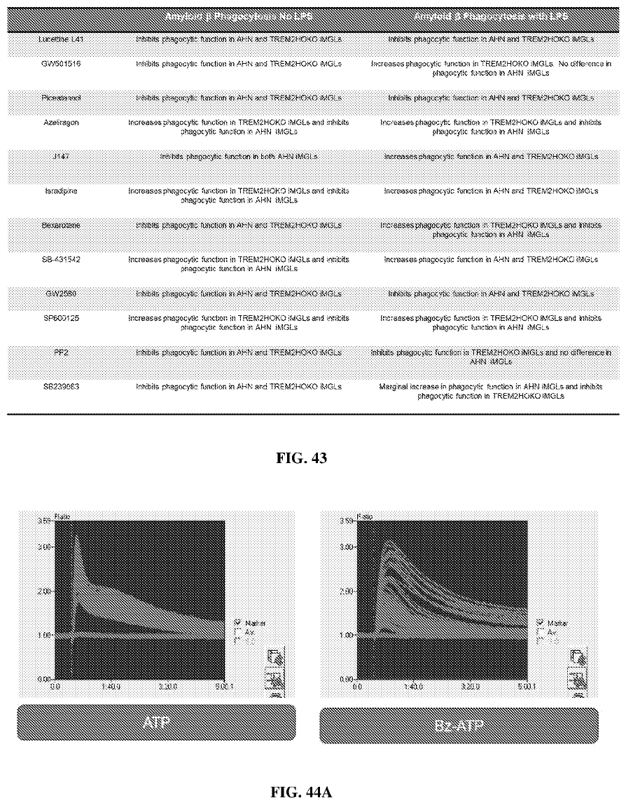

Cryopreserved microglia were thawed in maturation media and allowed to recover for 48 hrs before screening experiments were performed (FIG. 36). 5,000 microglia from TREM2 WT and TREM2 HOKO were plated per well of a 384 well plate in 40 uL media for 24hrs. In the first set (plate1) the cells were pre-treated with compounds at 1 μM final concentration. 24 hours post treating the cells with compounds, pHrodo labelled Amyloid-Beta was added to plate 1 at a final concentration of 1 μM and the phagocytosis was captured on an IncuCyteS3 for 96 hrs (FIGS. 37-39). In the second set (plate 2) the cells were plated for 24 hrs followed by treatment with lug/ml LPS at a final concentration of 1 μg/mL (FIGS. 40-42). 24 hours after exposure to LPS, and 48 hours after initial plating, pHrodo labelled Amyloid-Beta added to plate 2 at a final concentration of 1 μM. Cells were imaged using an IncuCyte once per hour for up to 96 hrs. Phagocytic data was captured as Total Red Object Integrated Intensity X μM2/image. The final volumes for all treatments remained constant. The results of the screen are summarized in FIG. 43.

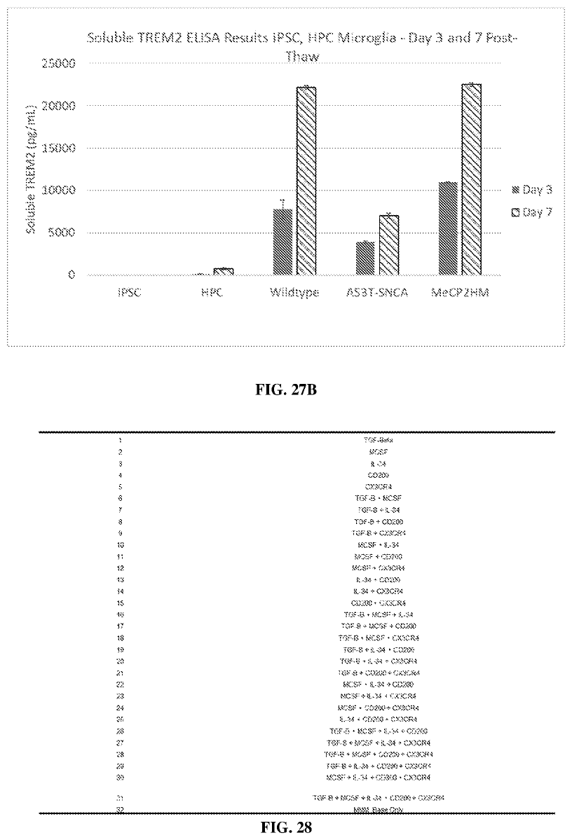

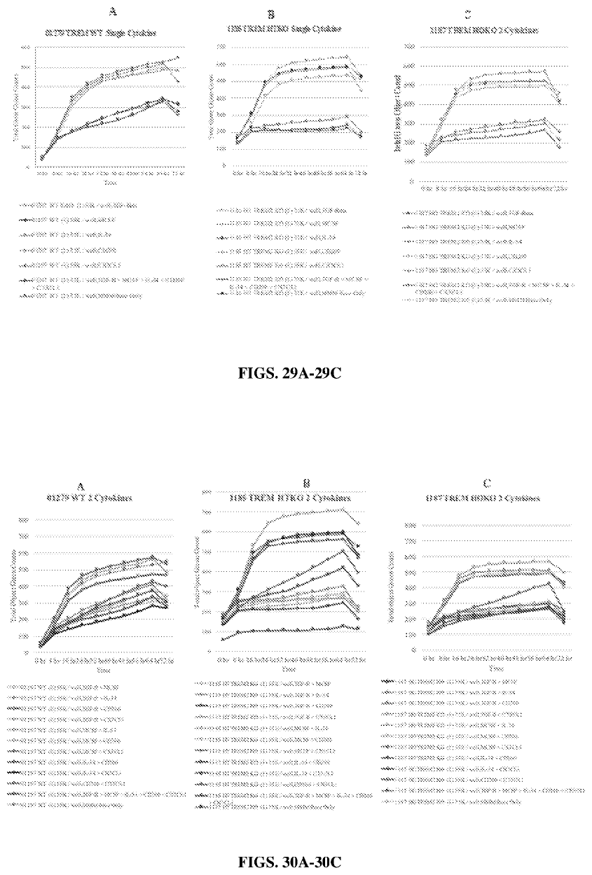

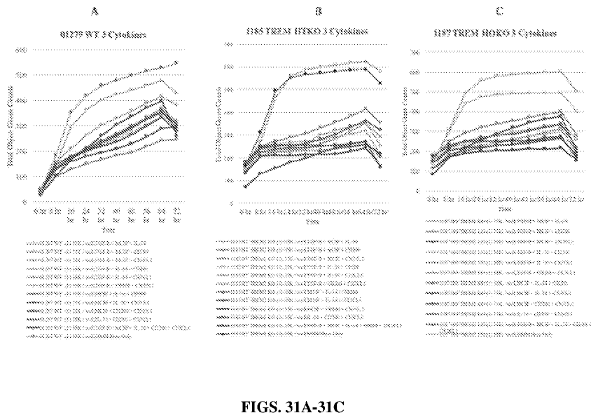

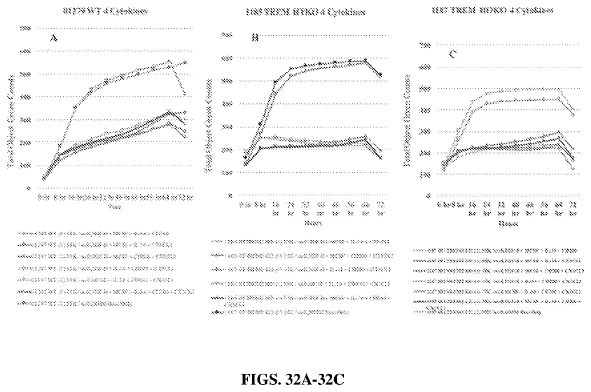

To understand the cytokines needed for microglia survival post thaw in the maturation medium a schematic matrix was planned with 32 different media formulations (FIG. 28). WT, 1185 HT TREM2 KO, 1187 HO TREM2 KO microglia were placed at a density of 15,000 viable cell in a 96 well plate in 250 μl of microglia base medium or MMM, or microglia base medium supplemented with a single cytokine (FIG. 29), two cytokines (FIG. 30), three cytokines (FIG. 31), or four cytokines (FIG. 32) in the maturation media. The kinetics of cell survival was captured on the IncuCyte system. NucGreen Dead diluted to 2 drops/mL was added to all well containing cells with various media compositions to capture the number of dead cells with time. The images were captured every 8 hours and the experiment continued for 72 hours without any intermittent feeds. The intensity of the NucGreen Dead quantifies the dead cells in the cultures.

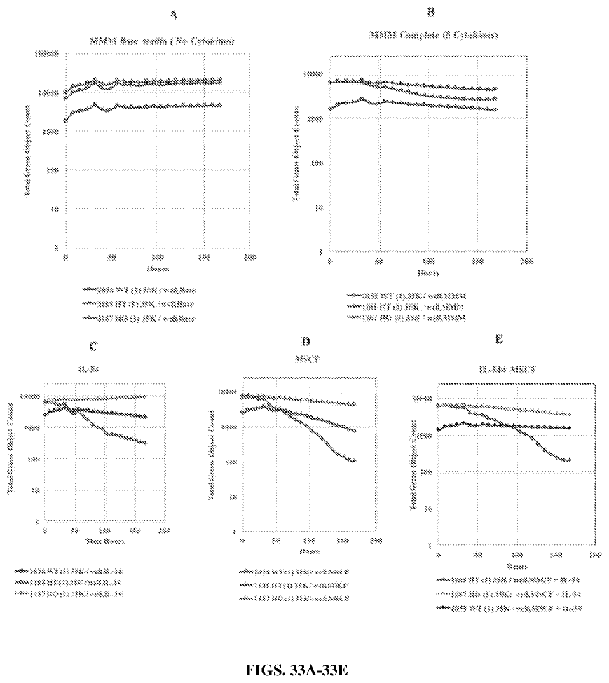

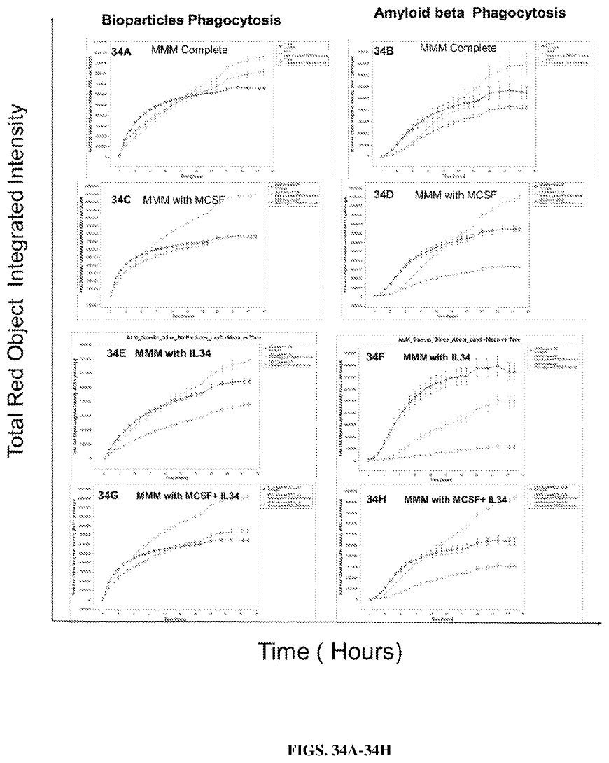

WT, 1185 HT TREM2 KO, 1187 HO TREM2 KO microglia were placed at a density of 15,000 viable cell in a 96 well plate in 250 μl of microglia base medium (FIG. 33A) or MMM (FIG. 33B), microglia base medium supplemented with IL-34 (26C), microglia base medium supplemented with IL-34 (FIG. 33D), microglia base medium supplemented with MCSF (FIG. 33D), or base medium supplemented with only IL-34 (FIG. 33C) or MSCF or a combination of IL-34 and MCSF (FIG. 33E). The kinetics of cell survival was captured on the IncuCyte system. NucGreen Dead diluted to 2 drops/mL was added to all well containing cells with various media compositions to capture the number of dead cells with time. The images were captured every 8 hours and the experiment continued for 7 days without any intermittent feeds. The intensity of the NucGreen Dead quantifies the dead cells in the cultures

Functional characterization was assessed on WT, 1185 HT TREM2 KO, 1187 HO TREM2 KO microglia, cryopreserved on day 23 with pHrodo Red labelled bacterial BioParticles and pHrodo Red Amyloid beta. WT, 1185 HT TREM2 KO, 1187 HO TREM2 KO microglia were plated at a density of 15,000-30,000 viable cells/cm² in a 96 well plate in 250 μl of MMM (FIGS. 33A-B) or MDM base (AKA microglia base medium) supplemented only MSCF (FIGS. 33C-D) or IL-34 (FIGS. 33E-F) or a combination of IL-34 and MCSF (FIGS. 33G-H) for three days post thaw. The cells were treated with diluted 1 μg/well of opsonized or non-opsonized pHrodo Bioparticles (Thermo Fisher #A10010, 2 mg per vial; stored at −20C) (FIGS. 33A,C,E,G) or pHrodo Amyloid beta (FIGS. 33B,D.F.H). The plate was placed on the IncuCyte and image of the phagocytosis were taken at various time points up to 30 hrs. WT as well as engineered microglia revealed phagocytic function post thaw. The kinetics and the efficiency of phagocytosis varied between the WT, 1185 HT TREM2 KO, 1187 HO TREM2 KO microglia.

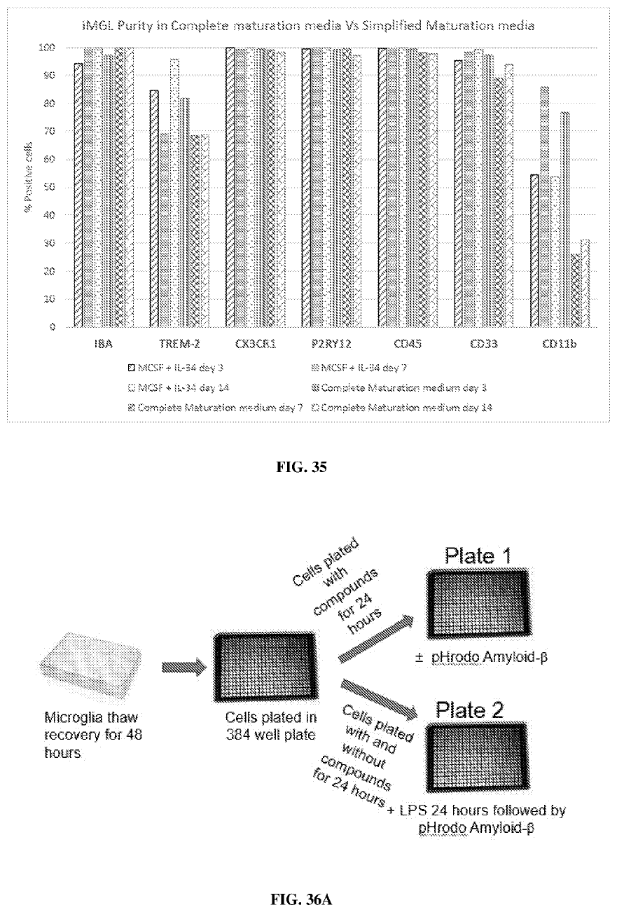

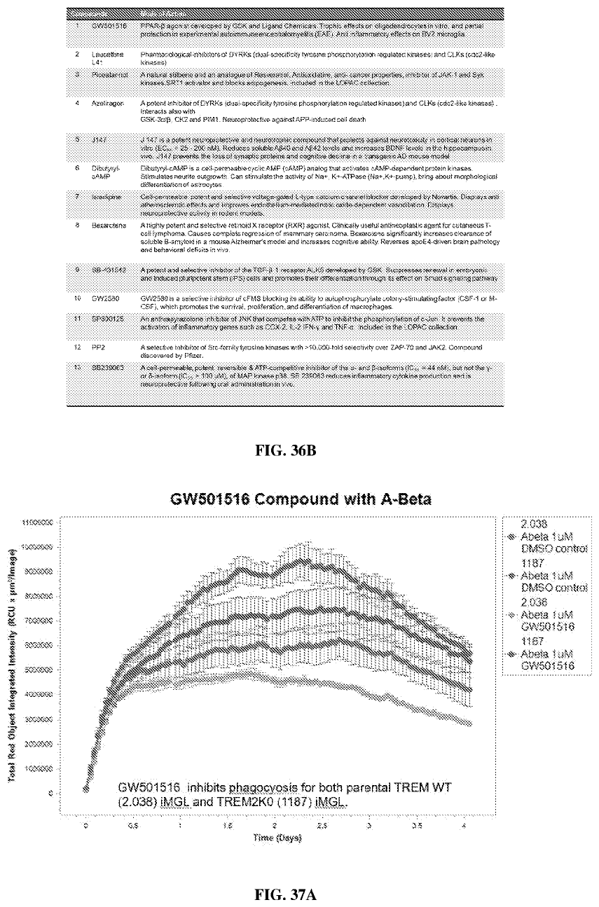

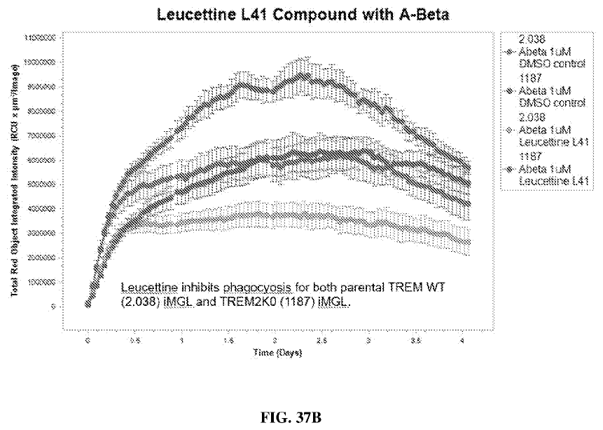

Post thaw purity of day 23 cryopreserved wild type (WT) microglia was determined in the presence of MMM or microglia base medium supplemented with combinations of two critical (IL-34, MSCF) cytokines in the maturation media (FIG. 35). The purity was quantified at day 3, 7 and day 14 post thaw by harvesting the cells and the purity of CD45, CD33, TREM2, CD11b, CX3CR1, P2RY12, TMEM119, IBA was determined at the end of the differentiation process by harvesting the cells and staining the cells for cell surface and intra cellular staining of markers by flow cytometry. Cryopreserved microglia retain viability, purity in maturation media supplemented with MSCF and IL-34. This simplified media will be valuable for co culture applications of cryopreserved microglia with neurons and astrocytes for developing brain organoid models to study the contribution of many SNPs and mutations associated with neurodegeneration.

### Example 8—Generation of Disease Associated Microglia From Patient Derived iPSCs

Recent genetic studies have shown that polymorphisms in several microglial-enriched genes are associated with altered risk of developing Alzheimer's disease (AD), Parkinson's disease (PD) and several neural degenerative diseases. Summarizing the list of risk associated SNPs from GWAS studies a panel of end stage microglia were generated from donors exhibiting mutations in TREM2, CD33, and ABCA7, along APOE isoforms.

Cryopreserved microglia from patient derived iPSC provide an in vitro tool to create a more accurate model to understand complex interactions between human microglia, neurons, astrocytes in a 2D or 3D organoid systems and mimic neurogenerative diseases (McQuade et al., 2019).

Generation of HPCs from episomally reprogrammed AHN and disease specific iPSCs: Episomally reprogrammed iPSC generated from normal as well as disease specific donors were acclimatized to hypoxia for at least 5-10 passages using E8/MATRIGEL™ before banking the source material for differentiation towards hematopoietic cells and subsequently to microglia. The genotypes of the panel of iPSCs is described in Table 4. The iPSC derived from all donors were karyotyped, and iPSC banks were made to initiate HPC differentiation via the 3D HPC differentiation protocol was performed as described in example 5. Microglia were generated by thawing cryopreserved HPCs and placing the cells in a 23-day differentiation process as described in Example 5. Cryopreserved day 23 microglia derived from various donors were thawed and stained for the presence of microglia specific markers. The cells were stained for cell surface expression of CD45, CD33, TREM2, and CD11b as well as intracellular expression of PU.1, IBA, P2RY12, TREM2 and TMEM119 proteins by flow cytometry. Table 5 summarizes the purity obtained across all AHN and disease associated microglia (DAM). The results demonstrate the generation of highly pure microglia from a panel of healthy and disease specific donors with no changes in the differentiation protocol.

TABLE 4
Generation of cryopreserved Microglia from a panel of Apparently
Healthy Normal (ANH) and Disease Associated Microglia (DAM)

| | | | APOE | APOE | APOE | TREM2 | CD33 |
|---|---|---|---|---|---|---|---|
| Phenotype | Genotype | Sex | rs429358 | rs7412 | genotype | Rs7593268 | Rs12459419 |
| AHN | N/A | Female | T/T | C/C | 3/3 | C/C | T/T |
| AHN | N/A | Female | T/T | C/C | 3/3 | C/C | T/T |
| AHN | N/A | Female | T/T | C/C | 3/3 | C/C | T/T |
| AHN | TREM2 R47H | Female | T/C | C/T | 3/3 | C/T | C/C |
| AHN | N/A | Male | T/T | C/C | 3/3 | C/C | C/C |
| AHN | CD33 | Female | T/C | C/T | 2/4 | C/C | T/T |
| AD | APOE 4/4 | Female | C/C | C/C | 4/4 | C/C | C/C |
| AD | APOE 4/4 | Female | C/C | C/C | 4/4 | C/C | C/C |
| AD | APOE 4/4 | Female | C/C | C/C | 4/4 | C/C | C/T |
| AD | APOE 4/4 | Female | C/C | C/C | 4/4 | C/C | T/T |
| AHN | ABCA7 G1527A | Male | T/T | C/C | 3/3 | C/C | C/T |
| AD | APOE 2/4 | Female | T/T | C/C | 2/4 | C/C | C/T |

TABLE 5
Overview of purity of microglia apparently healthy normal
(ANH) and Alzheimer's Disease (AD) donor samples.

| | | | | Percentage Purity Microgila Markers post thaw | | |
|---|---|---|---|---|---|---|
| CIRM ID | Phenotype | Genotype | Sex | CD33 | CD45 | TREM2 |
| CW13060BB1 | AHN | N/A | Female | 92 | 100 | 99 |
| CW13093AA1 | AHN | N/A | Female | 91 | 100 | 86 |
| CW13105FF1 | AHN | N/A | Female | 98 | 100 | 98 |
| CW13006DD1 | AHN | TREM2 R47H | Female | 99 | 100 | 98 |
| CW13061EE1 | AHN | N/A | Male | 99 | 100 | 94 |
| CW13042BB1 | AHN | CD33 | Female | 90 | 100 | 96 |
| CW13030EE1 | AD | APOE E4/E4 | Female | 98 | 100 | 98 |
| CW13032CC1 | AD | APOE E4/E4 | Female | 100 | 100 | 99 |
| CW13074AA1 | AD | APOE E4/E4 | Female | 97 | 100 | 94 |
| CW13098AA1 | AD | APOE E4/E4 | Female | 89 | 100 | 93 |
| CW13037AA1 | AHN | ABCA7 G1527A | Male | 98 | 100 | 96 |
| CW13005AA1 | AD | APOE E2/E4 | Female | 97 | 100 | 94 |

| | Percentage Purity Microgila Markers post thaw | | | | | | | S. aureus/ |
|---|---|---|---|---|---|---|---|---|
| CIRM ID | P2RY12 | TMEM119 | CX3CR1 | IBA1 | CD11c | CD11b | PU.1 | Phagocytosis |
| CW13060BB1 | 81 | 100 | 100 | 82 | 100 | 100 | 93 | Yes |
| CW13093AA1 | 93 | 99 | 99 | 98 | 99 | 97 | 94 | Yes |
| CW13105FF1 | 83 | 100 | 99 | 83 | 100 | 99 | 93 | Yes |
| CW13006DD1 | 90 | 100 | 99 | 98 | 100 | 100 | 99 | Yes |
| CW13061EE1 | 96 | 100 | 99 | 99 | 97 | 94 | 94 | Yes |
| CW13042BB1 | 84 | 99 | 98 | 95 | 100 | 80 | 99 | Yes |
| CW13030EE1 | 98 | 99 | 99 | 98 | 97 | 90 | 93 | Yes |
| CW13032CC1 | 100 | 100 | 99 | 97 | 100 | 99 | 97 | Yes |
| CW13074AA1 | 93 | 100 | 99 | 97 | 97 | 95 | 91 | Yes |
| CW13098AA1 | 81 | 100 | 99 | 95 | 100 | 75 | 98 | Yes |
| CW13037AA1 | 80 | 100 | 96 | 95 | 99 | 99 | 98 | Yes |
| CW13005AA1 | 82 | 99 | 98 | 97 | 100 | 98 | 97 | Yes |

Levels of soluble TREM2 (sTREM2) protein secreted by microglia post thaw were quantified from conditioned media collected from microglia generated from a panel of iPSC donors using a Simple Step ELISA (AbCam) (FIG. 45). Microglia generated from apparently healthy normal donors and disease specific donors were thawed and plated at the same density in maturation media in a 96 well Primaria plate. The spent media was collected at Day 3 post thaw and day 7 post thaw. The cultures were half fed with fresh maturation media on day 3 and Day 5 post thaw.

The data revealed a difference in the levels of soluble TREM2 between various samples of microglia derived from donor exhibiting an R47H genotype revealed the highest level of Soluble TREM levels at day 3 post thaw and it stayed high even 7 days post thaw. Although this donor was asymptomatic and hence classified as AHN, the iPSC derived microglia secreted high levels of sTREM. This data is in agreement with the high levels sTREM2 observed in the cerebrospinal fluid of Alzheimer disease patients and associated with this mutation status (Cheng et al., 2016). This data is a powerful example to use iPSC derived microglia in designing predictive kits of screening for the onset of neurodegenerative diseases. Young donors with an APOE4/4/genotype exhibiting the onset of AD revealed a high level of soluble TREM compared to older donors with the same genotype. The presence of SNPs in CD33 or ABCA7 gene did not seem to enhance the release of soluble TREM in the supernatant media. AHN Donor 12068 revealed a high level of soluble TREM at 3- and 7-days post thaw.

Neuroinflammation contributes to progression and pathogenesis of many neurodegenerative diseases. Resident microglia and astrocytes in the brain release cytokines which can play both pro-inflammatory and anti-inflammatory roles in the brain depending on the stimulus and microenvironment. This fluctuation between the pro- and anti-inflammatory profiles has been associated with the onset of AD and other neurodegenerative diseases. To quantify the levels of cytokines and chemokines released by disease associated microglia (DAM), cryopreserved AHN and DAM microglia were thawed into MDM medium and plated on to Primaria 96 well plate at 50,000 cells/well. Cells were plated for three days prior to starting stimulations with 100 ng/ml LPS to check M1 mediated response or with 10 ng/ml IL-4 along with 10 uM dBu-cAMP to trigger an M2 specific response. Stimulations were performed in triplicate for 24 hours. Supernatant was spun down to remove cells and debris and immediately placed at −20° C. The supernatants were analyzed on a multiplex Luminex assay. The results of this multiplex Luminex assay is captured as a heat map in FIG. 27C.

Chemokines CCL1, CCL2, CCL3, CCL4, CCL8, CCL11, CCL13,CCL17, CCL18, CCL20, CCL22, CCL24, serve as chemoattractants and mediate the recruitment of myeloid cells, granulocytes, lymphoid cells or neural precursor cells into inflamed areas, enhance phagocytic response and are generally upregulated in Alzheimer's Disease (AD) or Multiple Sclerosis. In response to LPS or dBu-cAMP, microglia derived from APOE E4/E4, APOE E2/E4, TREM2 R47H, ABCA7 G1527A and CD33 (bearing the rs429358 SNP) derived microglia released a higher level of all these analytes compared to AHN lines. The fold increase varied from 0.1 to as high as 7 fold between various microglial genotypes. This data from disease associated microglia supports an earlier finding demonstrating an increase in CCL2 and CCL5 expression in AD brain samples. CCL2 expression in the brain and cerebrospinal fluid (CSF) has been reported as a reliable predictor of AD severity by Westin et al.

APOE E4/E4, APOE E2/E4, TREM2 R47H, ABCA7 G1527A and CD33 (bearing the rs429358 SNP) derived microglia released marginally elevated levels of soluble CD163, a marker of inflammation and inflammatory diseases related to M2 polarization. The release of sCD163 prevents monocyte hyperactivation and reduces the secretion of pro-inflammatory cytokines TNF-alpha, IL-1beta, IL-6 and IL-8. A similar trend was observed with Chitinase-3 which plays a role in tissue remodeling during neural inflammation (Melief et al., 2012); Minett et al., 2016).

PD-L1 and its receptor, PD-1, elicit inhibitory signals that regulate the balance between T-cell activation, tolerance, and immune-mediated tissue damage. In response to LPS, APOE E4/E4 derived microglia had no increase compared to AHN derived microglia. TREM2 R47H, APOE E2/E4, and CD33 (bearing the rs429358 SNP) derived microglia displayed an increase compared to AHN lines. In response to IL-4+dBu-cAMP, APOE E4/E4, TREM2 R47H, ABCA7 G1527A displayed a marginal increase over AHN lines, while APOE E2/E4 derived microglia had a 7-fold increase compared to AHN derived microglia.

APOE E4/E4, APOE E2/E4, TREM2 R47H, ABCA7 G1527A and CD33 (bearing the rs429358 SNP) derived microglia released marginally elevated levels of soluble Fractalkine, a soluble chemokine promoting chemotaxis, survival and enhancing neuroprotection by decreasing levels of TNF-alpha and Nitic Oxide during neuroinflammation.

APOE E4/E4, APOE E2/E4, and ABCA7 G1527A derived microglia released high levels of CXCL1/GRO alpha in response to both LPS and dBu-cAMP whereas TREM2 R47H and CD33 derived microglia revealed a marginal increase in the levels of this cytokine released, implying a correlation of this analyte with the APOE and ABCA7 genotypes. A recent study showed that CXCL1 could contribute to inflammation response in the development of AD, but not as a potential genetic factor conferring the predisposition to AD in the pathogenesis of this disease. Under physiological conditions CX3CR1 maintains microglial homeostasis by limiting their activation. The high levels of this cytokine released post stimulation indicates the onset of a rescue mechanism by CX3CR1 to preserve homeostatic function in disease associated microglia associated with APOE or ABCA7 G1527A genotypes. Alternately, the high levels of CX3CR1 secreted by APOE E4/E4 and ABCA7 G1527A activated microglia could be a signal to promote neuronal degeneration (Atagi et al., 2015; Wolfe et al., 2018).

APOE E4/E4 and APOE E2/E4 derived microglia released high levels of IL-6 in response to both LPS and IL4/dBu-cAMP while all other genotypes secreted similar levels of IL-6 as AHN. IL-6 secretion attracts granulocytes, promotes a cell-mediated humoral Th2 response and trigger inflammation. This mechanism would again support greater neural inflammation associated with the APOE E4/E4 genotype.

ABCA7 G1527A derived microglia released high levels of IL-1 beta and IL-1 alpha in response to LPS, while the other genotypes secreted comparable levels to AHN microglia.

APOE E4/E4, APOE E2/E4, and ABCA7 G1527A derived microglia also released high levels of IL-8/CXCL8 in response to LPS than dBu-cAMP. ABCA7 G1527A derived microglia released high levels of IL-8 implying the onset of a pro-inflammatory response contributing to brain injury.

Microglia also secrete proteolytic enzymes and matrix metalloproteinase that may eliminate AP deposition and limit AD process thus performing a neuroprotective role in AD. APOE E4/E4, APOE E2/E4, TREM2 R47H, ABCA7 G1527A and CD33 (bearing the rs429358 SNP) derived microglia released marginally elevated levels of MMP-9 and MMP-12 compared to AHN derived microglia.

R47H TREM2 derived microglia released over 7 times more IL-12 p70 in comparison to AHN derived microglia when stimulated by LPS or M1 stimulation. On the other hand, APOE E2/E4 released over 7 times more IL-12 p70 in comparison to AHN derived microglia in response to IL4+dBu-cAMP or M2 stimulation. The other genotypes revealed a marginal increase in the levels of IL-12 secretion. Primary microglia produce IL-12 in the brain to control immune responses during infection or in Th1 cell-mediated autoimmune diseases of the CNS. The increased levels of IL-12 by R47H TREM2 derived microglia implies a strong activation of cytotoxic activity of NK-cells and T cells.

Finally, APOE E4/E4, APOE E2/E4, ABCA7 G1527A derived microglia released similar or marginally high levels of IL-13, IL-18, IL-23 and Alpha

Synuclein levels, implying a lack of correlation of these analytes with the above-mentioned genotypes.

Neuroinflammation is an important contributor to Alzheimer's disease (AD) pathogenesis and progression. A combination of several inflammatory mediators creates a unique signature associated with a particular SNP mutation. iPSC derived microglia from disease specific donors can be used to determine the key signature cytokines associated with various microglial SNPs and mutation associated genotypes.

The phagocytic function of microglia is important to preserve the neuroprotective effect. Microglia mediated phagocytosis can be impaired by disease specific SNPs or mutations which in turn can affect critical homeostatic mechanisms in the brain. The phagocytic function of Disease associated microglia (DAM) was evaluated in the presence of pHrodo labelled bacterial S aureus and amyloid beta to compare the role of disease associated SNPs on phagocytic function of microglia. This function can be used for high throughput screening applications.

Among these microglial expressed disease associated genes, sequence variants in the gene encoding the triggering receptor expressed on myeloid cells 2 (TREM2) and APO E isoforms are associated with an extraordinarily increased risk for AD. APOE is the primary cholesterol carrier in the brain, and plays an essential role in lipid trafficking, cholesterol homeostasis, and synaptic stability. Anti-ApoE immunotherapy inhibits amyloid accumulation and deposition further supporting a role of ApoE in AP aggregation and clearance. ApoE expression has been shown to be significantly upregulated in disease- associated microglia. ApoE4/E4 isoform has been shown to intrinsically affect microglia physiology by upregulating motility and phagocytic behavior in vitro. ApoE4/E4 overexpression has been shown to reduced uptake of Abeta in contrast to the other isoforms. The role of ApoE2, the third most common major ApoE isoform, in neurodegeneration has been shown to delay the onset of disease in familial AD. ApoE-isotype specific effects on iPSC derived microglia function have not been thoroughly investigated to date.

TREM2 senses lipids and mediates myelin phagocytosis. Loss-of-function (LOF) variants of TREM2, have been associated with an increased amyloid plaque seeding, reduced amyloid clustering and with an added interaction with ApoE triggers signaling cascade leading to reduced microglial clustering and ApoE accumulation in amyloid plaques with impairment of function.

The phagocytic function of Disease associated microglia (DAM) was evaluated in the presence of bacterial S. aureus and amyloid beta to compare the role of disease associated SNPs on phagocytic function of microglia.

On the same lines, ATP-binding cassette transporter A7 (ABCA7) has been identified as a susceptibility factor of late onset Alzheimer disease in genome-wide association studies. ABCA7 has been shown to mediate phagocytosis and affect membrane trafficking. ABCA7 is strongly associated with AD. Phagocytic clearance of amyloid-beta is impaired in Abca7-/- mice. iPSCs derived from patients a possessing a missense variant associated with G1527A substitution in ABCA7 provide BCA7 plays a role in the regulation of Abeta homeostasis in the brain to altered phagocyte function.

CD33 is an immunomodulatory receptor linked to Alzheimer's disease (AD) susceptibility via regulation of phagocytosis in microglia. TREM2 interacts downstream of CD33 in modulating microglial physiology and metabolism, thus iPSC derived microglia expressing WT and CD33 rs3865444 SNP can be used to validate the role of CD33 associated with impaired phagocytic function (Caldeira et al., 2017).

Cryopreserved AHN and DAM microglia were thawed, cultured for three days in maturation media and exposed to pHrodo labeled Amyloid Beta and pHrodo S aureus. The kinetics of phagocytosis were measured using the IncuCyte Live-Cell Analysis System. The total red object integrated intensity was used to quantify the functional response.

TREM2 R47H microglia and ABCA7-G1527A microglia revealed a strong phagocytotic ability to bacterial S. Aureus compared to AHN microglia. CD33 (bearing the rs429358 SNP) microglia revealed comparable phagocytotic ability to bacterial S. Aureus compared to AHN microglia. TREM2 R47H microglia, ABCA7-G1527A microglia and CD33 (bearing the rs429358 SNP) microglia revealed reduced intensity of phagocytosis in the presence of Amyloid Beta in comparison to AHN microglia.

CW13030EE1 APOE 4/4 displayed the higher intensity of phagocytosis with S. aureus and Amyloid Beta compared to AHN microglia. CW13098AA1 APOE 4/4 displayed a lower intensity of phagocytosis with both S. aureus and Amyloid Beta compared to AHN microglia. CW13005AA1 APOE 2/4 microglia revealed strong phagocytic ability to Amyloid Beta and a reduced phagocytosis to S. aureus compared to AHN derived microglia. CW13074AA1 APOE 4/4 microglia exhibited a similar trend of phagocytosis for S. aureus as AHN microglia and a slightly enhanced phagocytosis to Amyloid Beta to AHN microglia cell lines.

A comprehensive understanding of the genetic alterations that target homeostatic, pro-inflammatory and anti-inflammatory microglial subtypes can provide novel biological insights and facilitate target prioritization for immunomodulatory therapeutic approaches for neurodegenerative diseases.

### Example 9—Additional Characterization of Microglia

Extracellular nucleotides, such as ATP and ADP, are known to elicit receptor mediated pathways termed as the “purinergic signaling” pathway. Physiological processes such as tissue homeostasis, wound healing, neurodegeneration, immunity, inflammation and cancer are modulated by purinergic signaling. Extracellular ATP and P2 receptors are important for the microglial activation mechanism. P2X receptors are ionotrophic receptors that bind to ATP or their derivatives. One of the P2Y receptors is a G-protein coupled receptor that responds to ADP. Under pathological conditions, nucleotides such as ATP are released or leaked from injured cells and function as “find me” or “eat me” signals to evoke process extension, chemotaxis, and phagocytosis by microglia. P2 receptor activation also induces cytokine production from microglia, including interleukin-1b (IL-1b), interleukin-6 (IL-6), and tumor necrosis factor alpha (TNFα). Such pro-inflammatory mediators have been shown to dynamically change G protein coupled receptor (GPCR) expression and function in astrocytes.

The purinergic receptor response of microglia was characterized. Microglia were thawed in microglia differentiation media and reconstituted to a cell suspension of 200,000 cells/well. 15 μl of cell suspension of microglia was added per well of a 384 well plate. The cells were treated with different doses (0-1000 nM) of BzATP and ADP. The response to BzATP and ADP was also measured in the presence of AZ11645373 (P₂X₇ antagonist) and AZD1283 (a potent antagonist of the P2Y₁₂ receptor). For all treatments 10 ul of a 4× stock of the compound or inhibitor was added to the cells. The cells were exposed to the compounds in the presence or absence of inhibitors for 30 minutes before the assay. One bottle of FLPR Calcium-6 was reconstituted to 11 ml with Assay Buffer B and 15 μl of the dye solution added to the cell suspension in the presence of the compounds. The cells were incubated at 37C for 1.5 hours and imaging was performed on the FDSS μCELL system.

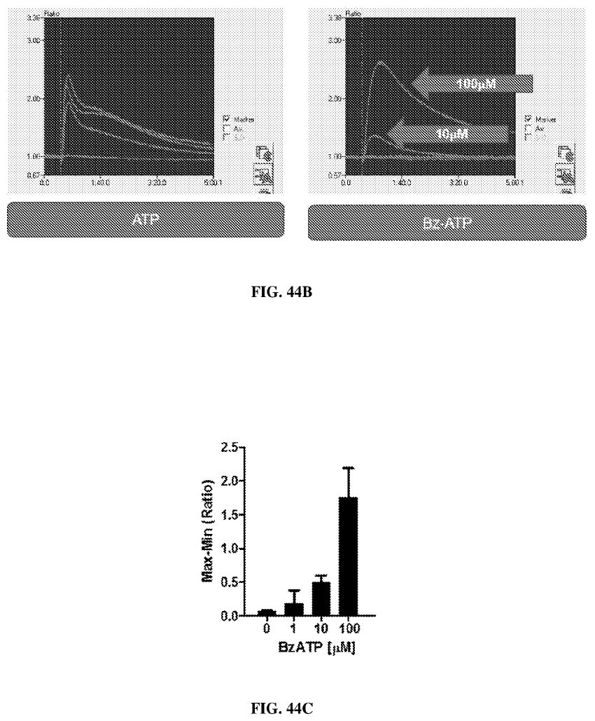

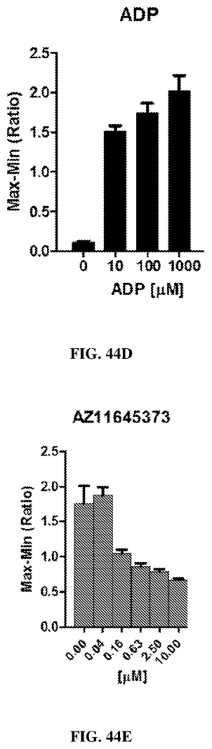

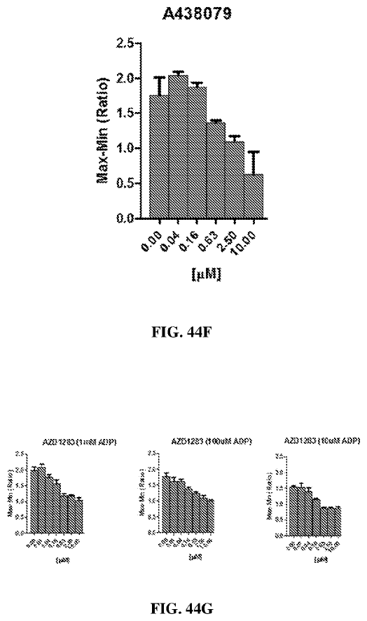

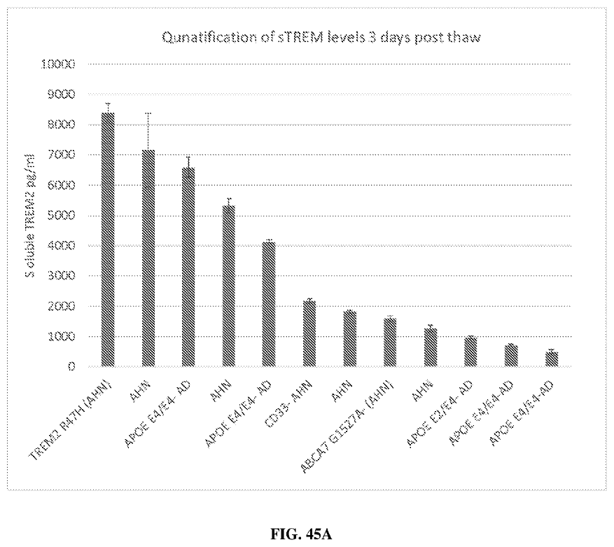

FIG. 44A shows microglia with ATP/BzATP All Traces and FIG. 44B shows microglia with ATP/BzATP Sample Traces. FIGS. 44C-F show the response of microglia to BzATP, ADP, BzATP in the presence of P₂X₇ antagonist AZ11645373, and BzATP in the presence of P₂X₇ antagonist A438079 FIG. 44G shows a dose dependent response to demonstrate functional ADP dependent response in microglia in the presence of AZD1283.

### Example 10—Generation of Neural Precursor Cells from iPSC

The successful development of in vitro disease models is dependent on the availability of large quantities of end stage lineages derived from patient derived iPSCs. Neural presursor cells (NPCs) are self-renewing progenitors with the ability to generate neurons and glia (Breunig et al., 2011). There are many established protocols with varying efficiencies for generating NPCs from primary neural cells and iPSCs (Shi et al., 2012a, Shi et al., 2012b). Most of the recent protocols rely on the inhibition of the SMAD signaling pathway. The present application describes a simple protocol to generate NPCs across different iPSCs lines utilizing the spontaneous drift of iPSC towards ectoderm without using the dual SMAD inhibition pathway. The rationale to generate this cell type is to pair it with iPSC derived microglia to generate long term co-culture assays to mimic human brain development and complex intercellular interactions between neural lineages, microglia, endothelial cells, pericytes, and astrocytes in a dish derived from normal and disease specific iPSC cells.

In this study, multiple episomally reprogrammed iPSC lines were maintained on MATRIGEL™, Laminin, or Vitronectin coated plates and E8 media. The iPSCs were maintained under hypoxic conditions before the onset of differentiation to generate neural precursor cells. To initiate neural precursor differentiation, iPSCs were harvested and seeded at 15K/cm² on MATRIGEL™, Laminin or Vitronectin plates using E8 media in the presence of rock inhibitor. The cells were placed in fresh E8 media for the next 48 hours in the absence of rock inhibitor. The next step involved the preconditioning step comprising placing iPSC cultures in DMEMF12 media supplemented with 3 uM CHIR for 72 hours with a daily change in media under normoxic conditions. Cells were harvested at the end of the preconditioning step and either replated back in a 2D format on MATRIGEL™, Laminin or Vitronectin plates at 30K/cm² or generated 3D aggregates using Ultra low attachment plates or spinner flasks at a density of 0.3 million cells per ml in the presence of a rock inhibitor. The cultures were fed every other day with E6 media supplemented with N2 for the next 8 days under normoxic conditions. On day 14 of differentiation the cultures were harvested and individualized using TrypLE. The cells were stained for the presence of Tra-162, CD56, CD15 by cell surface staining for flowcytometry and for the presence of Sox1, Nestin, β3 Microglobulin, and Pax-6 expression by intracellular staining for flow cytometry. The different steps involved in the generation of NPCs are outlined in FIG. 45A. The emergence of NPC markers at different days of differentiation across three iPSC lines is summarized in FIG. 45B. CD56 was used as the marker for NPCs derived by this method. The cells were cryopreserved using CS10 and they retained purity and proliferation potential post thaw. NPCs were placed in downstream differentiation protocols to generate Astrocytes and Pan neurons.

Astrocytes were differentiated from NPCs according to the protocol outlined by Julia et al. Day 14 NPC cells were plated onto MATRIGEL™ coated 6 well plates at 15k/cm 2 in Science Cell Astrocyte Medium. The plates were given a full media exchange every two days. Every 6 days, or when cultures were ˜90% confluent, the plates were harvested using Accumax and replated onto MATRIGEL™ coated 6 well plates at 15k/cm². The cultures were fed and replated as described above for 4 passages. At the end of 4 passages the culture was stained for surface markers, CD44 and glutamate aspartate transporter (GLAST), and intracellular markers, Glial fibrillary acidic protein (GFAP), Excitatory amino acid transporter 1 (EAAT1), Glutamine Synthetase (GS), Aquaporin 4 (AQP4), and S100 calcium-binding protein B (S100β) (FIG. 45C).

Cortical glutamatergic neurons were generated from NPCs using the protocol developed by Slosarek et al day 14 NPC were plated in E6 medium supplemented with 1 μM cyclic AMP, 10 ng/ml brain-derived neurotrophic factor (BDNF), and 10 ng/ml glial-derived neurotrophic factor (GDNF)) for 30 days. The medium was subsequently changed to cortical neural differentiation medium (E6 medium, 1 μM cyclic AMP, 10 ng/ml BDNF, 10 ng/ml GDNF, 100 ng/ml Insulin-like growth factor-I, and 2% B27 supplement) for an additional 30 days (Brennand et al., 2011). Cortical glutamatergic neurons were observed between day 14-36 for different iPSC lines. The purity of the neural cultures was confirmed by staining for the presence of β3 Tubulin, MAP2 expression.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Atagi et al., J Biol Chem. 290(43): 26043-50, 2015.
Brennand et al., 2011
Caldeira et al., Front Aging Neurosci. 9: 277, 2017.
Cheng et al., Clin Chim Acta. 463: 88-95, 2016.
International Patent Publication No. WO 02/016536
International Patent Publication No. WO 03/016496
International Patent Publication No. WO 98/30679
International Patent Publication No. WO 98/53058
International Patent Publication No. WO 98/53059
International Patent Publication No. WO 98/53060
Julia et al. Stem cell reports. 9(2): 600-614, 2017.
McQuade et al., J Mol Biol. 431(9): 1805-17, 2019.
Melief et al., Glia. 60(10): 1506-17, 2012.
Minett et al., J Neuroinflammation. 13(1): 135, 2016.
PCT Publication No. 2012/149484
Rustenhoven et al., Trends In Pharmacological Sciences, 38(3), 291-304, 2017.
Slosarek et al. Cell Rep. 24(9): 2248-2260, 2018.
U.S. Patent Application No. 12/715,136
U.S. Pat. No. 6,140,081
U.S. Pat. No. 6,453,242
U.S. Pat. No. 6,534,261
U.S. Pat. No. 6,617,152
U.S. Patent No. 8,372,642
U.S. Patent Publication No. 2002/0076747
U.S. Patent Publication No. 2005/0064474
U.S. Patent Publication No. 2005/0260186
U.S. Patent Publication No. 2006/0104968
U.S. Patent Publication No. 2006/0188987
U.S. Patent Publication No. 2007/0218528
U.S. Patent Publication No. 2011/0301073
U.S. Patent Publication No. 2011/0301073
U.S. Patent Publication No. 2011/0301073.
Westin et al. PLoS ONE. 7:e30525, 2012.
Winkler et al., Brain pathology (Zurich, Switzerland), 24(4), 371-386, 2014.
Wolfe et al., Int J Mol Sci. 20(1), 2018.

What is claimed is:

1. An in vitro method for differentiating induced pluripotent stem cells (iPSCs) comprising:
(a) culturing the iPSCs on a charged surface in the absence of extracellular matrix proteins; and
(b) differentiating the iPSCs to endothelial cells, mesenchymal stem cells (MSCs), or hematopoietic precursor cells (HPCs).

2. The method of claim 1, wherein the charged surface is a positively charged surface.

3. The method of claim 2, wherein the positively charged surface is an amine surface or Poly L Lysine surface.

4. The method of claim 2, wherein the positively charged surface comprises nitrogen-containing functional groups.

5. The method of claim 1, wherein the charged surface is a negatively charged surface.

6. The method of claim 5, wherein the negatively charged surface is a carboxyl surface.

7. The method of claim 5, wherein the negatively charged surface comprises oxygen-containing functional groups.

8. The method of any of claims 1-7, wherein the charged surface comprises positively charged groups and negatively charged groups.

9. The method of claim 8, wherein the positively charged groups are nitrogen-containing groups and the negatively charged groups are oxygen-containing groups.

10. The method of any of claims 1-9, wherein the charged surface is a polymeric surface.

11. The method of claim 10, wherein the polymeric surface is a polystyrene surface.

12. The method of any of claims 1-11, wherein the iPSCs are cultured in serum free defined media.

13. The method of claim 12, wherein differentiating comprises culturing in the presence of blebbistatin or a ROCK inhibitor.

14. The method of claim 13, wherein the ROCK inhibitor is H1152.

15. The method of any of claims 1-14, wherein the culturing is carried out in the absence of laminin, fibronectin, vitronectin, MATRIGEL™, tenascin, entactin, thrombospondin, elastin, gelatin, and/or collagen.

16. The method of any of claims 1-15, further comprising engineering the iPSCs to have disrupted expression of TREM2 prior to step (a).

17. The method of claim 16, wherein engineering comprises using a TAL nuclease to introduce indels in exon 2 of TREM2.

18. The method of any of claims 1-17, further comprising engineering the iPSCs to have disrupted expression of Methyl-CpG Binding Protein 2 (MeCP2).

19. The method of claim 18, wherein the disrupted expression of MeCP2 is further defined as a truncated mutant of MeCP2 protein.

20. The method of any of claims 1-19, further comprising engineering the iPSCs to have disrupted expression of Alpha-synuclein (SNCA).

21. The method of claim 20, wherein the disrupted expression of SCNA is due to a missense point mutation.

22. The method of claim 21, wherein the missense point mutation is A53T.

23. The method of any of claims 1-22, wherein the method comprises differentiating the progenitor cells to endothelial cells.

24. The method of claim 23, wherein step (a) comprises culturing on an amine surface to generate progenitor cells and step (b) comprises culturing on a carboxyl surface in the presence of endothelial differentiation media to produce endothelial cells.

25. The method of claim 24, wherein the endothelial cells are positive for CD31.

26. The method of any of claims 1-25, further comprising differentiating the endothelial cells to brain microvascular endothelial cells (BMECs).

27. The method of any of claims 1-25, further comprising differentiating the endothelial cells to lymphatic endothelial cells.

28. The method of any of claims 1-25, wherein the method comprises differentiating the progenitor cells to MSCs.

29. The method of claim 28, wherein differentiating comprises culturing the progenitor cells on an amine surface in the presence of MSC media.

30. The method of claim 29, wherein the MSCs are positive for CD73, CD44, and CD105.

31. The method of claim 30, wherein at least 90% of the differentiated cells are positive for CD73.

32. The method of claim 28, further comprising differentiating the MSCs to pericytes.

33. The method of claim 32, wherein the MSCs are cultured in the presence of pericyte medium in the absence of extracellular proteins.

34. The method of claim 32, wherein the pericytes are positive for NG2, PDGFRβ, and CD146.

35. The method of any of claims 1-25, wherein the method comprises differentiating the progenitor cells to HPCs.

36. The method of claim 35, further comprising differentiating the HPCs to microglia.

37. The method of claim 36, wherein differentiating comprises culturing the HPCs on a neutrally charged surface or ultralow attachment surface in the presence of microglia differentiation media.

38. The method of claim 37, wherein the microglia differentiation media comprises IL34, TGF, and MCSF.

39. The method of claim 37, wherein differentiating comprises culture at normoxia.

40. The method of claim 37, wherein differentiating is for 20-25 days.

41. The method of claim 36, wherein the microglia are positive for CD45, CD11b, and CD33.

42. The method of claim 41, wherein at least 50% of the differentiated cells are positive for CD11b.

43. The method of claim 41, wherein at least 90% of the differentiated cells are positive for CD33.

44. The method of any of claims 1-43, wherein step (b) of the method does not comprise purification of the cells.

45. The method of claim 44, wherein purification is further defined as performing MACS.

46. The method of any of claims 1-45, wherein the method is good-manufacturing practice (GMP) compliant.

47. The method of any of claims 1-46, wherein the method is performed under hypoxic conditions.

48. The method of any of claims 1-47, wherein the iPSCs are human

49. The method of any of claims 1-48, wherein one or more of steps (a)-(d) are performed under xeno-free conditions, feeder-free conditions, and/or conditioned-media free conditions.

50. The method of any of claims 1-49, wherein each of steps (a)-(d) are performed under xeno-free conditions, feeder-free conditions, and/or conditioned-media free conditions.

51. The method of claim 49 or 50, wherein each of steps (a)-(d) are performed under defined conditions.

52. A composition comprising a microglia cell population at least 90% positive for TREM2, P2RY12, TMEM119, IBA-1, and/or CX3CR1, wherein the microglia cell population is differentiated from iPSCs.

53. The composition of claim 52, wherein the microglia cell population produced by the method of any one of claims 1-65.

54. The composition of claim 52, wherein the microglia cell population is generated from disease associated iPSC donors with TREM2, APOE, CD33, BIN, ABCA7, SNPS or genotypes associated with neurodegeneration.

55. The composition of claim 52, wherein the microglia cell population has disrupted expression of TREM2.

56. The composition of claim 53, wherein the disrupted expression of TREM2 is further defined as a homozygous knockout of TREM2 expression.

57. The composition of any of claims 52-56, wherein the microglia cell population has disrupted expression of Methyl-CpG Binding Protein 2 (MeCP2).

58. The composition of claim 57, wherein the disrupted expression of MeCP2 is further defined as a truncated mutant of MeCP2 protein.

59. The composition of any of claims 52-59, wherein the microglia cell population has disrupted expression of Alpha-synuclein (SNCA).

60. The composition of claim 59, wherein the disrupted expression of SCNA is due to a missense point mutation.

61. The composition of claim 60, wherein the missense point mutation is A53T.

62. A method for screening a test compound comprising introducing the test compound to a microglia cell population of any of claims 52-56.

63. The method of claim 56, wherein the microglia cell population is further introduced to amyloid-beta.

64. The method of claim 56, wherein the microglia cell population is further introduced to LPS.

65. A composition comprising a pericyte cell population produced by the method of any one of claims 1-65.

66. A blood-brain-barrier model comprising microglia, pericytes, and BMECs produced by the method of any of claims 1-65.

67. A method for generating microglia comprising:
(a) differentiating iPSCs to HPCs; and
(b) selecting for CD34-positive cells from the HPCs; and
(c) culturing the HPCs in Microglia Differentiation Medium, thereby generating a population of microglia.

68. The method of claim 67, wherein the HPCs are differentiated according to claim 1.

69. The method of claim 67, wherein selecting comprises sorting for CD34-positive cells.

70. The method of claim 69, wherein sorting comprises using CD34 magnetic beads.

71. The method of claim 67, wherein the method does not comprise sorting HPCs for CD43-positive cells.

72. The method of claim 67, wherein the Microglia Differentiation Medium comprises IL- 34, TGFβ1, or M-CSF.

73. The method of claim 72, wherein the Microglia Differentiation Medium comprises 200 ng/mL IL-34, 100 ng/mL TGFβ1, and 50 ng/mL M-CSF.

74. The method of claim 67, wherein the cells are fed with Microglia Differentiation Medium every 48 hours.

75. The method of claim 67, wherein the method does not comprise ECM proteins.

76. The method of claim 67, wherein at least 90% of the cells in the population of microglia are TREM-positive.

77. The method of claim 67, wherein at least 10% of the HPCs are differentiated to microglia.

78. The method of claim 67, wherein the culturing of step (b) is performed in a 96 well format.

79. The method of claim 67, wherein the culturing of step (b) is performed on a charged surface.

80. The method of claim 79, wherein the charged surface is positively charged.

81. The method of claim 80, wherein the positively charged surface is an amine surface.

82. The method of claim 79, wherein the charged surface is negatively charged.

83. The method of claim 82, wherein the negatively charged surface is a carboxyl surface.

84. The method of claim 79, wherein the charged surface comprises positively charged groups and negatively charged groups.

85. The method of claim 84, wherein the positively charged groups are nitrogen-containing groups and the negatively charged groups are oxygen-containing groups.

86. The method of claim 67, further comprising maturing the population of microglia in media comprising CD200 and/or fractalkine.

87. The method of claim 86, further comprising cryopreserving the microglia.

88. The method of claim 67, wherein the HPCs are differentiated from iPSCs engineered to have disrupted expression of TREM2.

89. The method of claim 88, wherein engineering comprises using TAL nucleases.

90. The method of claim 87, wherein the cryopreserved microglia retain phagocytic function towards pHrodo bacterial particles.

91. The method of claim 87, wherein the cryopreserved microglia can mature post thaw and respond to stimulants and secrete interleukins, chemokines and immune modulating ligands in the supernatant media

92. An in vitro method for producing neural precursor cells (NPCs) from iPSCs comprising:
(b) pre-conditioning iPSCs in media comprising a glycogen synthase kinase 3 (GSK3) inhibitor;
(b) differentiating the iPSCs to NPCs, wherein the method does not comprise inhibition of SMAD signaling.

93. The method of claim 92, wherein the iPSCs are maintained under hypoxic conditions prior to step (a).

94. The method of claim 92 or 93, wherein the iPSCs are seeded in the presence of a ROCK inhibitor and then cultured in the absence of a ROCK inhibitor prior to step (a).

95. The method of any of claims 92-94, wherein the GSK3 inhibitor is CHIR99021, BIO, or SB-216763.

96. The method of claim 95, wherein the GSK3 inhibitor is CHIR99021.

97. The method of claim 96, wherein the CHIR99021 is added to the media at a concentration of 3 μM.

98. The method of any of claims 92-97, wherein the pre-conditioning is for 2-4 days.

99. The method of any of claims 92-98, wherein the pre-conditioning is for 3 days.

100. The method of any of claims 92-99, wherein the iPSCs of step (a) and/or step (b) are cultured on an extracellular matrix (ECM) protein-coated surface.

101. The method of any of claims 92-100, wherein the ECM protein is MATRIGEL™, laminin, or vitronectin.

102. The method of any of claims 92-101, wherein the ECM protein is laminin or vitronectin.

103. The method of any of claims 92-102, wherein steps (a) and (b) are performed under normoxic conditions.

104. The method of any of claims 92-103, wherein differentiating comprises culturing the iPSCs on an ECM protein-coated surface.

105. The method of claim 104, wherein the ECM protein is laminin or vitronectin.

106. The method of any of claims 92-103, wherein differentiating comprises culturing the iPSCs on an ultralow attachment plate or spinner flask in the presence of a ROCK inhibitor.

107. The method of any of claims 92-106, wherein step (b) is performed for 5 to 10 days.

108. The method of any of claims 92-107, wherein step (b) is performed for 7 days.

109. The method of any of claims 92-108, further comprising detecting expression of Tra-162, CD56, CD15, Sox1, Nestin, Microglobulin, and/or Pax-6 in the NPCs.

110. The method of any of claims 92-109, wherein at least 70% of the NPCs are positive for CD56.

111. The method of any of claims 92-110, further differentiating the NPCs to astrocytes or neurons.

112. A method of screening for a neurodegenerative disease comprising detecting a level of soluble TREM2 in microglia conditioned media.

113. The method of claim 112, wherein detecting comprises performing an ELISA.

114. The method of claim 112 or 113, wherein the microglia are derived from isogenically engineered iPSCs or a donor expressing disease-associated SNPs or mutations.

115. The method of any of claims 112-114, wherein the microglia are produced by the method of any of claim 1-51 or 67-91.

116. The method of any of claims 112-114, wherein an increased level of soluble TREM2 as compared to a control detects a neurogenerative disease.

117. The method of any of claims 112-116, wherein the neurodegenerative disease is Alzheimer's disease or multiple sclerosis.

118. A method for performing high-throughput screening to identify a therapeutic agent comprising contacting microglia produced by the method of any of claim 1-51 or 67-91 with a plurality of candidate agents and measuring cytokine and/or chemokine levels and/or amyloid beta phagocytic function.

119. The method of claim 118, wherein the microglia are cryopreserved microglia derived from isogenically engineered iPSC lines, microglia derived from a donor expressing disease associated SNPs, or microglia derived from a donors expressing mutation associated with neurodegeneration.

120. The method of claim 118 or 119, wherein the cytokines and/or chemokines are selected from the group consisting of IL6, IL10, IL3, TNFα, IL13, CCL2/MCP-1, CCL20/MIP-3α, CCL4/MIP-1β, CCL5/RANTES, CX3CL1/Fractalkine, CXCL1/GROα, CXCL10/IP- 10, CXCL2/GROβ, and IL-8/CXCL8.

121. A co-culture comprising microglia produced by the method of any of claim 1-51 or 67-91 and endothelial cells, pericytes, astrocytes, and/or neural precursor cells.

122. Use of the co-culture of claim 121 to mimic human brain development.
